SEQUENCE LISTING (93 sequences)

SEQ ID NO: 1 - DNA, 44 bp, Artificial Sequence
  Synthetic Construct
catgagttgtagggagggatttatgcctaaaacccaaagtgctg

SEQ ID NO: 2 - DNA, 60 bp, Artificial Sequence
  Synthetic Construct
ataacgcgtctgcagggcaggcggccgccgcgcgcgttaaagcttatccattgtctcaaa

SEQ ID NO: 3 - DNA, 29 bp, Artificial Sequence
  Synthetic Construct
ggcccccactatctcgaccttgaactacc

SEQ ID NO: 4 - DNA, 23 bp, Artificial Sequence
  Synthetic Construct
agctcgggccccaaataatgatt

SEQ ID NO: 5 - DNA, 22 bp, Artificial Sequence
  Synthetic Construct
aaatccctccctacaactcatg

SEQ ID NO: 6 - DNA, 56 bp, Artificial Sequence
  Synthetic Construct
atgcctgcagatgcaggtcgaccatatgaaacagtagacattagcagataaattag

SEQ ID NO: 7 - DNA, 43 bp, Artificial Sequence
  Synthetic Construct
tccaacgcgttggaaataatcaacattactgcaactagaattg

SEQ ID NO: 8 - DNA, 25 bp, Artificial Sequence
  Synthetic Construct
ctattgctcctttctttttctgcag

SEQ ID NO: 9 - DNA, 38 bp, Artificial Sequence
  Synthetic Construct
atgcctgcaggataacttcgtatagcatacattatacg

SEQ ID NO: 10 - DNA, 40 bp, Artificial Sequence
  Synthetic Construct
agatcgcgcgcgaaacagtagacattagcagataaattag

SEQ ID NO: 11 - DNA, 42 bp, Artificial Sequence
  Synthetic Construct
agatgggcccttcaaatcttgtatatctaggtaagtatatac

SEQ ID NO: 12 - DNA, 60 bp, Artificial Sequence
  Synthetic Construct
gtatatctccttcttgagatctgttgactttgtataccattccgttgtaaataaatgatc

SEQ ID NO: 13 - DNA, 56 bp, Artificial Sequence
  Synthetic Construct
cccatatgtatatctccttctcccatatgtatatctccttcttgagatctgttgac

SEQ ID NO: 14 - DNA, 50 bp, Artificial Sequence
  Synthetic Construct
catgggtatatctccttctcccatatgtatatctccttctcccatatgta

SEQ ID NO: 15 - DNA, 55 bp, Artificial Sequence
  Synthetic Construct
agatgggcccacgcgtcgcgcgcgttcaatttattcaattgtaaaataaacgacg

SEQ ID NO: 16 - DNA, 59 bp, Artificial Sequence
  Synthetic Construct
ccattccgttgtaaataaatgatcttaacccattttaattaattaattaaattaattag

SEQ ID NO: 17 - DNA, 49 bp, Artificial Sequence
  Synthetic Construct
agatgggcccacgcgtcgcgcgcgttcgttagtgttagtctagatctag

SEQ ID NO: 18 - DNA, 54 bp, Artificial Sequence
  Synthetic Construct
ccattccgttgtaaataaatgatcttaaatatgatactctataaaaatttgctc

SEQ ID NO: 19 - DNA, 53 bp, Artificial Sequence
  Synthetic Construct
agatgggcccacgcgtcgcgcgcgagtcttactaaaacgaaatgaaattaatg

SEQ ID NO: 20 - DNA, 55 bp, Artificial Sequence
  Synthetic Construct
ccattccgttgtaaataaatgatcttacaaaataaatatgatggaagtgaaagag

SEQ ID NO: 21 - DNA, 55 bp, Artificial Sequence
  Synthetic Construct
gtcaacagatctcaagaaggagatatacccatgagtatgaaaaccttgccaaaag

SEQ ID NO: 22 - DNA, 42 bp, Artificial Sequence
  Synthetic Construct
agatgcggccgcacgcgtttaatatcttccaggtcgatgcac

SEQ ID NO: 23 - DNA, 48 bp, Artificial Sequence
  Synthetic Construct
gtcaacagatctcaagaaggagatatacccatggcgtcaacgcagagg

SEQ ID NO: 24 - DNA, 41 bp, Artificial Sequence
  Synthetic Construct
agatgcggccgcacgcgttcaatccgcatgggaggcattag

SEQ ID NO: 25 - DNA, 53 bp, Artificial Sequence
  Synthetic Construct
gtcaacagatctcaagaaggagatatacccatgagcgcttataacggccaagg

SEQ ID NO: 26 - DNA, 45 bp, Artificial Sequence
  Synthetic Construct
agatgcggccgcacgcgtttacggcttttgaatcaacagttcagc

SEQ ID NO: 27 - DNA, 22 bp, Artificial Sequence
  Synthetic Construct
atggctcgtgaagcggttatcg

SEQ ID NO: 28 - DNA, 28 bp, Artificial Sequence
  Synthetic Construct
ttatttgccaactaccttagtgatctcg

SEQ ID NO: 29 - DNA, 26 bp, Artificial Sequence
  Synthetic Construct
accatgcaattgaaccgattcaattg

SEQ ID NO: 30 - DNA, 29 bp, Artificial Sequence
  Synthetic Construct
tgtatactctttcatatatatagcgcaac

SEQ ID NO: 31 - DNA, 25 bp, Artificial Sequence
  Synthetic Construct
atgtcaccacaaacagagactaaag

SEQ ID NO: 32 - DNA, 26 bp, Artificial Sequence
  Synthetic Construct
ttacttatccaaaacgtccactgctg

SEQ ID NO: 33 - DNA, 642 bp, Synechococcus elongatus
atgggcgttgagctgcgcagttacgtctacctcgacaatttgcaacggcaacacgcctcctatatcggtacagtcgccacaggctttttgaccctgccaggggatgcctcggtctggattgaaatctccccgggtattgaaatcaaccggatgatggacatcgccctcaaggcggcggtggtgcggcctggagtgcagttcatcgaacgcctctacggcttgatggaagtccacgccagtaatcaaggcgaagtccgtgaagcgggacgtgccgttctctctgctctgggactgacggagcgcgatcgcctcaaacccaaaattgtctccagccaaatcatccgcaatattgatgctcaccaagcgcagctgatcaaccggcagcgccgtggtcaaatgctgctggctggtgaaaccctctacgtcctcgaagtgcaaccggcggcttatgcagcgctagcagccaacgaagcggaaaaggcggcgttgatcaacatcctgcaagtcagtgcgattggcagttttgggcgactctttttgggtggggaggagcgcgacatcattgctggctcgcgggctgctgtagcagcactggaaaacctgtcgggacgtgagcatcccggcgatcgctcgcgggagtag

SEQ ID NO: 34 - protein, 213 aa, Synechococcus elongatus
MGVELRSYVYLDNLQRQHASYIGTVATGFLTLPGDASVWIEISPGIEINRMMDIALKAAVVRPGVQFIERLYGLMEVHASNQGEVREAGRAVLSALGLTERDRLKPKIVSSQIIRNIDAHQAQLINRQRRGQMLLAGETLYVLEVQPAAYAALAANEAEKAALINILQVSAIGSFGRLFLGGEERDIIAGSRAAVAALENLSGREHPGDRSRE

SEQ ID NO: 35 - DNA, 831 bp, Synechococcus elongatus
atgtcggcttctcttcccgcctattctcagcctcgcaatgcaggtgcactaggggtcatttgtacccgtagttttccagcggttgtcggcactgcagacatgatgctcaagtcggccgatgtcacattgatcggctatgagaaaacaggctcgggcttttgtacagcaatcatccggggtggctatgccgacatcaagctggctcttgaggctggcgtagcgacagctcgtcagtttgagcagtacgtttccagcactattctgccgcggcctcaaggcaacctcgaagccgtgttgccgattagccggcggctctcccaagaagccatggccacgcgatcgcatcagaatgttggcgcgattgggctaattgagaccaatgggttccctgctttggttggagcagccgatgccatgctcaaatcggctaacgtcaagctgatttgttatgagaaaacgggcagcggtctctgtactgcgatcgtgcaaggcacggtttctaatgtgaccgttgcggtcgaagccgggatgtatgccgctgagcggatcggccagctcaacgcaatcatggtcattcccagaccgctagacgacttgatggacagcttgcctgagccgcagtcggatagcgaagcagcccagccactccaattaccgctgcgggttcgcgaaaaacaaccgctgttggagctaccggaactcgaacggcagccgatcgcgatcgaagcaccgcgacttttagcagaagagcgacagtctgcgttggaattggctcaagagacaccgctcgccgagcccttagagctccccaatcctcgtgatgatcagtga

SEQ ID NO: 36 - protein, 276 aa, Synechococcus elongatus
MSASLPAYSQPRNAGALGVICTRSFPAVVGTADMMLKSADVTLIGYEKTGSGFCTAIIRGGYADIKLALEAGVATARQFEQYVSSTILPRPQGNLEAVLPISRRLSQEAMATRSHQNVGAIGLIETNGFPALVGAADAMLKSANVKLICYEKTGSGLCTAIVQGTVSNVTVAVEAGMYAAERIGQLNAIMVIPRPLDDLMDSLPEPQSDSEAAQPLQLPLRVREKQPLLELPELERQPIAIEAPRLLAEERQSALELAQETPLAEPLELPNPRDDQ

SEQ ID NO: 37 - DNA, 309 bp, Synechococcus elongatus
atgcctattgcggttggaatgatcgagaccctgggcttcccggctgttgtggaagcagctgacgcgatggtcaaagcagcgcgtgtcacgctggttggctatgagaagattggcagcggccgcgtcactgtcattgtccggggagacgtttcggaagttcaagcttctgtctctgcgggtctcgattcggcgaaacgggttgccggtggtgaagtgctgtcgcaccacatcattgcgcgtccccacgagaacttggaatacgttctcccgattcgctacaccgaagctgttgaacaattccgcatgtaa

SEQ ID NO: 38 - protein, 102 aa, Synechococcus elongatus
MPIAVGMIETLGFPAVVEAADAMVKAARVTLVGYEKIGSGRVTVIVRGDVSEVQASVSAGLDSAKRVAGGEVLSHHIIARPHENLEYVLPIRYTEAVEQFRM

SEQ ID NO: 39 - DNA, 300 bp, Synechococcus elongatus
atgcgcattgctaaggttcgcggaaccgtagtcagtacctacaaagagcccagcctgcaaggggtaaagttcttggttgttcagttcttggatgaggctggacaggcacttcaagagtatgaggttgctgctgacatgattggcgctggcgttgacgagtgggtgttgattagccgcggcagtcaagcgcgccatgtgcgcgattgtcaggaacgaccggttgatgcagctgtcattgccatcatcgatacggtcaacgtggaaaaccgctccgtctacgacaaacgcgagcacagctaa

SEQ ID NO: 40 - protein, 99 aa, Synechococcus elongatus
MRIAKVRGTVVSTYKEPSLQGVKFLVVQFLDEAGQALQEYEVAADMIGAGVDEWVLISRGSQARHVRDCQERPVDAAVIAIIDTVNVENRSVYDKREHS

SEQ ID NO: 41 - DNA, 972 bp, Synechococcus elongatus
tctgcttataacggacaaggtcgattaagttctgaagtaattactcaagttcgaagtttgttaaaccaaggatatcgaattggaactgaacatgctgataagagacgatttagaactagttcttggcaaccttgtgctcctattcaatctactaatgagagacaggtattgtctgaacttgaaaattgtctttctgaacatgaaggtgaatacgttcgattgttaggaattgataccaatactagatctcgtgtttttgaagctttaattcaacgacctgatggttcggttcctgaatcgttaggatctcaacctgtggcagtagcttcaggtggaggtcgacaatcatcttatgcaagtgtatctggaaatttatctgctgaagtagttaataaagtacgtaatctattagctcaaggatatcgaattggtacagaacacgcagacaaaagacgatttcgtacttcttcatggcagtcatgcgcaccaatccagagttctaacgagcgtcaagttcttgctgagcttgaaaactgcttaagtgagcatgagggagagtacgttagattacttggtatcgatactgcttctagaagtcgtgttttcgaagcacttatacaagatccacaaggacctgtaggttctgctaaagctgcagccgctcctgtatcttcagctactccaagttctcatagttatacttctaatggatctagttcgagcgatgtcgctggacaggttcgaggtcttctagcacagggttaccgtataagtgctgaagtagctgataagcgtagattccaaacaagttcttggcaaagtttacctgctcttagtggacagtctgaagcaactgtattgcctgctttggagtcaattcttcaagaacacaaaggtaagtatgtacgtcttattgggattgaccctgcagctcgtcgtcgagtagctgagttgttgattcagaagccgtaa

SEQ ID NO: 42 - protein, 323 aa, Synechococcus elongatus
SAYNGQGRLSSEVITQVRSLLNQGYRIGTEHADKRRFRTSSWQPCAPIQSTNERQVLSELENCLSEHEGEYVRLLGIDTNTRSRVFEALIQRPDGSVPESLGSQPVAVASGGGRQSSYASVSGNLSAEVVNKVRNLLAQGYRIGTEHADKRRFRTSSWQSCAPIQSSNERQVLAELENCLSEHEGEYVRLLGIDTASRSRVFEALIQDPQGPVGSAKAAAAPVSSATPSSHSYTSNGSSSSDVAGQVRGLLAQGYRISAEVADKRRFQTSSWQSLPALSGQSEATVLPALESILQEHKGKYVRLIGIDPAARRRVAELLIQKP

SEQ ID NO: 43 - DNA, 1614 bp, Synechococcus elongatus
ccttctccaacaactgtacctgttgctactgctggtagattggctgaaccttatattgatcctgctgctcaagttcatgcaattgctagtataatcggcgacgtacgtattgcagctggagtaagagttgcagctggagtttcgattcgtgctgatgaaggagcaccatttcaagtaggtaaagaatctattcttcaagagggagctgtaattcatggattggaatatggtcgtgtattgggtgatgatcaagcagactattccgtctggataggccagcgagtagctattactcataaagcacttattcatggaccagcttatcttggagatgattgttttgtaggtttccgatctaccgtatttaacgctcgtgttggagccggttcggtaatcatgatgcacgcccttgtccaagacgtagagattcctcctggtagatatgttccttctggagcaattatcacaactcaacagcaagctgatcgactacctgaagttcgtcctgaagatcgagaatttgctagacatataattggatcacctccagtaattgtaagatctactccagcagctactgctgattttcactcaacaccaactccttctccacttcgtccatcgtctagtgaggcaacaactgtatctgcttataacggacaaggtcgattaagttctgaagtaattactcaagttcgaagtttgttaaaccaaggatatcgaattggaactgaacatgctgataagagacgatttagaactagttcttggcaaccttgtgctcctattcaatctactaatgagagacaggtattgtctgaacttgaaaattgtctttctgaacatgaaggtgaatacgttcgattgttaggaattgataccaatactagatctcgtgtttttgaagctttaattcaacgacctgatggttcggttcctgaatcgttaggatctcaacctgtggcagtagcttcaggtggaggtcgacaatcatcttatgcaagtgtatctggaaatttatctgctgaagtagttaataaagtacgtaatctattagctcaaggatatcgaattggtacagaacacgcagacaaaagacgatttcgtacttcttcatggcagtcatgcgcaccaatccagagttctaacgagcgtcaagttcttgctgagcttgaaaactgcttaagtgagcatgagggagagtacgttagattacttggtatcgatactgcttctagaagtcgtgttttcgaagcacttatacaagatccacaaggacctgtaggttctgctaaagctgcagccgctcctgtatcttcagctactccaagttctcatagttatacttctaatggatctagttcgagcgatgtcgctggacaggttcgaggtcttctagcacagggttaccgtataagtgctgaagtagctgataagcgtagattccaaacaagttcttggcaaagtttacctgctcttagtggacagtctgaagcaactgtattgcctgctttggagtcaattcttcaagaacacaaaggtaagtatgtacgtcttattgggattgaccctgcagctcgtcgtcgagtagctgagttgttgattcagaagccg

SEQ ID NO: 44 - protein, 538 aa, Synechococcus elongatus
PSPTTVPVATAGRLAEPYIDPAAQVHAIASIIGDVRIAAGVRVAAGVSIRADEGAPFQVGKESILQEGAVIHGLEYGRVLGDDQADYSVWIGQRVAITHKALIHGPAYLGDDCFVGFRSTVFNARVGAGSVIMMHALVQDVEIPPGRYVPSGAIITTQQQADRLPEVRPEDREFARHIIGSPPVIVRSTPAATADFHSTPTPSPLRPSSSEATTVSAYNGQGRLSSEVITQVRSLLNQGYRIGTEHADKRRFRTSSWQPCAPIQSTNERQVLSELENCLSEHEGEYVRLLGIDTNTRSRVFEALIQRPDGSVPESLGSQPVAVASGGGRQSSYASVSGNLSAEVVNKVRNLLAQGYRIGTEHADKRRFRTSSWQSCAPIQSSNERQVLAELENCLSEHEGEYVRLLGIDTASRSRVFEALIQDPQGPVGSAKAAAAPVSSATPSSHSYTSNGSSSSDVAGQVRGLLAQGYRISAEVADKRRFQTSSWQSLPALSGQSEATVLPALESILQEHKGKYVRLIGIDPAARRRVAELLIQKP

SEQ ID NO: 45 - DNA, 1419 bp, Synechococcus elongatus
atgcctaaaacccaaagtgctgctggatataaagcaggagttaaagattataaacttacctattatactccagattatactccaaaagataccgatttacttgctgcatttcgattcagtcctcaaccaggagtaccagcagatgaagctggtgctgcaattgcagcagaaagttcaacaggaacttggactaccgtttggacagatcttctaaccgatatggatagatataaagggaaatgttatcatattgaaccagtacaaggagaagagaattcctattttgcttttattgcatatcctcttgatttgtttgaagaaggatcagttactaacattctaactagtatcgttggaaatgtatttggattcaaagctatacgatcactacgtttggaagatatacgtttcccagttgctttggttaaaactttccaagggcctccacatggaattcaagttgaaagagatttattaaacaagtatgggcgaccaatgcttggatgtacaattaagcctaaattagggctatctgctaaaaactatggacgtgctgtatatgagtgtttaagaggaggattagattttactaaagatgatgaaaatattaattcacaaccttttcaacgatggcgagatagatttctttttgttgccgatgccattcataaatcacaagccgagactggagaaattaagggacattatctaaatgtaaccgccccaacatgtgaagaaatgatgaagcgagctgaatttgctaaagaattgggtatgccaatcataatgcatgattttctaactgctggattcaccgccaatactactttagctaagtggtgtcgtgataatggtgtattacttcatatacatcgagcaatgcatgctgtaatagatagacaacgaaaccatggtattcattttcgtgttttagcaaaatgtcttcgattgagtggaggggatcatttgcattctgggactgttgtagggaaattggaaggagataaagcctcaactcttggatttgtagatctaatgcgagaagatcatatagaggcagatagaagtagaggtgtatttttcacccaagattgggctagtatgcctggggttcttcctgtagctagtggaggaattcatgtttggcacatgccagcactagtagaaatcttcggagatgattcagttttacaatttggtggaggaactctaggtcatccatggggaaatgcaccaggtgcaacagcaaatcgtgttgctttagaagcatgtgtacaagctcgtaatgagggtcgagatttatatagagaagggggagatatacttagagaggctggaaaatggtctccagaattggcagctgcccttgatctatggaaagaaataaagtttgaatttgagacaatggataagctttaa

SEQ ID NO: 46 - protein, 472 aa, Synechococcus elongatus
MPKTQSAAGYKAGVKDYKLTYYTPDYTPKDTDLLAAFRFSPQPGVPADEAGAAIAAESSTGTWTTVWTDLLTDMDRYKGKCYHIEPVQGEENSYFAFIAYPLDLFEEGSVTNILTSIVGNVFGFKAIRSLRLEDIRFPVALVKTFQGPPHGIQVERDLLNKYGRPMLGCTIKPKLGLSAKNYGRAVYECLRGGLDFTKDDENINSQPFQRWRDRFLFVADAIHKSQAETGEIKGHYLNVTAPTCEEMMKRAEFAKELGMPIIMHDFLTAGFTANTTLAKWCRDNGVLLHIHRAMHAVIDRQRNHGIHFRVLAKCLRLSGGDHLHSGTVVGKLEGDKASTLGFVDLMREDHIEADRSRGVFFTQDWASMPGVLPVASGGIHVWHMPALVEIFGDDSVLQFGGGTLGHPWGNAPGATANRVALEACVQARNEGRDLYREGGDILREAGKWSPELAAALDLWKEIKFEFETMDKL

SEQ ID NO: 47 - DNA, 336 bp, Synechococcus elongatus
atgagtatgaaaaccttgccaaaagaacgaagatttgaaaccttcagttatttacctcctctttctgatcgtcaaattgctgctcaaatcgaatatatgatagaacaaggttttcatccattgatagaatttaatgaacattcaaatccagaagaattctattggaccatgtggaaactacctttgtttgattgtaagtctccacaacaagtattggatgaggtacgagaatgtcgttccgagtatggtgattgttatattagagttgcaggatttgataacatcaaacaatgtcaaaccgttagtttcattgtgcatcgacctggaagatattaa

SEQ ID NO: 48 - protein, 111 aa, Synechococcus elongatus
MSMKTLPKERRFETFSYLPPLSDRQIAAQIEYMIEQGFHPLIEFNEHSNPEEFYWTMWKLPLFDCKSPQQVLDEVRECRSEYGDCYIRVAGFDNIKQCQTVSFIVHRPGRY

SEQ ID NO: 49 - DNA, 459 bp, Synechococcus elongatus
atggcgtcaacgcagagggcgaagccgatggagatgccccgcatcagtcgcgatacagcccgcatgttggtcaattacctgacctatcaagcggtctgtgtgattcgggatcaattggctgagacgaatccggccggtgcataccggctgcaggttttctcggctgagttctcctttcaggatggagaagcttacctagcagctctactcaaccacgatcgcgaattgggcctgcgggtgatgacagtacgggaacatttggccgagcatattctcgactacctgccggagatgacgatcgctcagatccaggaggcgaatattaatcatcgccgtgctttgcttgaacggctgacgggtcttggggcagagcctagcttgccggagaccgaggtgagcgatcgccccagtgactcagccactcctgatgatgcttctaatgcctcccatgcggattga

SEQ ID NO: 50 - protein, 152 aa, Synechococcus elongatus
MASTQRAKPMEMPRISRDTARMLVNYLTYQAVCVIRDQLAETNPAGAYRLQVFSAEFSFQDGEAYLAALLNHDRELGLRVMTVREHLAEHILDYLPEMTIAQIQEANINHRRALLERLTGLGAEPSLPETEVSDRPSDSATPDDASNASHAD

SEQ ID NO: 51 - DNA, 975 bp, Synechococcus elongatus
atgagcgcttataacggccaaggccgactcagttccgaagtcatcacccaagtccggagtttgctgaaccagggctatcggattgggacggaacatgcggacaagcgccgcttccggactagctcttggcagccctgcgcgccgattcaaagcacgaacgagcgccaggtcttgagcgaactggaaaattgtctgagcgaacacgaaggtgaatacgttcgcttgctcggcatcgataccaatactcgcagccgtgtttttgaagccctgattcaacggcccgatggttcggttcctgaatcgctggggagccaaccggtggcagtcgcttccggtggtggccgtcagagcagctatgccagcgtcagcggcaacctctcagcagaagtggtcaataaagtccgcaacctcttagcccaaggctatcggattgggacggaacatgcagacaagcgccgctttcggactagctcttggcagtcctgcgcaccgattcaaagttcgaatgagcgccaggttctggctgaactggaaaactgtctgagcgagcacgaaggtgagtacgttcgcctgctgggcatcgacactgctagccgcagtcgtgtttttgaagccctgatccaagatccccaaggaccggtgggttccgccaaagctgccgccgcacctgtgagttcggcaacgcccagcagccacagctacacctcaaatggatcgagttcgagcgatgtcgctggacaggttcggggtctgctagcccaaggctaccggatcagtgcggaagtcgccgataagcgtcgcttccaaaccagctcttggcagagtttgccggctctgagtggccagagcgaagcaactgtcttgcctgctttggagtcaattctgcaagagcacaagggtaagtatgtgcgcctgattgggattgaccctgcggctcgtcgtcgcgtggctgaactgttgattcaaaagccgtaa

SEQ ID NO: 52 - protein, 324 aa, Synechococcus elongatus
MSAYNGQGRLSSEVITQVRSLLNQGYRIGTEHADKRRFRTSSWQPCAPIQSTNERQVLSELENCLSEHEGEYVRLLGIDTNTRSRVFEALIQRPDGSVPESLGSQPVAVASGGGRQSSYASVSGNLSAEVVNKVRNLLAQGYRIGTEHADKRRFRTSSWQSCAPIQSSNERQVLAELENCLSEHEGEYVRLLGIDTASRSRVFEALIQDPQGPVGSAKAAAAPVSSATPSSHSYTSNGSSSSDVAGQVRGLLAQGYRISAEVADKRRFQTSSWQSLPALSGQSEATVLPALESILQEHKGKYVRLIGIDPAARRRVAELLIQKP

SEQ ID NO: 53 - DNA, 309 bp, Synechococcus elongatus
atgccaattgctgtcggaacgattcaaaccctcggatttccgccgattattgctgcggcagatgcgatggtcaaagcggctcgggtcaccatcacccagtatggattggcggaaagtgcccaattctttgtctcggtgcggggacctgtttcggaagtcgaaacggctgttgaagcagggttgaaagcagttgctgaaaccgaaggggcagagctgatcaattacatcgtcatcccgaatccacaagaaaacgtggaaacggtgatgccgatcgacttcacggctgaatccgagccctttcggtcttaa

SEQ ID NO: 54 - protein, 102 aa, Synechococcus elongatus
MPIAVGTIQTLGFPPIIAAADAMVKAARVTITQYGLAESAQFFVSVRGPVSEVETAVEAGLKAVAETEGAELINYIVIPNPQENVETVMPIDFTAESEPFRS

SEQ ID NO: 55 - DNA, 342 bp, Synechococcus elongatus
atgtctcagcaggcaattggctcgctggaaacgaagggctttcccccaatcttggcggcagctgatgccatggtcaaagctggccgaatcacgattgtgagctacatgcgggccggtagcgctcgctttgcagtcaacattcggggggatgtctcagaagtcaaaacggcgatggatgcgggcattgaagccgcgaaaaatacgcctggtggcaccctcgaaacgtgggtgatcatccctcgcccgcatgaaaacgtggaagcggtcttcccgatcggctttggcccagaagtggaacaatatcgactctctgccgaaggaactggtagtggccgccgttaa

SEQ ID NO: 56 - protein, 113 aa, Synechococcus elongatus
MSQQAIGSLETKGFPPILAAADAMVKAGRITIVSYMRAGSARFAVNIRGDVSEVKTAMDAGIEAAKNTPGGTLETWVIIPRPHENVEAVFPIGFGPEVEQYRLSAEGTGSGRR

SEQ ID NO: 57 - DNA, 819 bp, Synechococcus elongatus
atgcgcaagctcatcgaggggttacggcatttccgtacgtcctactacccgtctcatcgggacctgttcgagcagtttgccaaaggtcagcaccctcgagtcctgttcattacctgctcagactcgcgcattgaccctaacctcattacccagtcgggcatgggtgagctgttcgtcattcgcaacgctggcaatctgatcccgcccttcggtgccgccaacggtggtgaaggggcatcgatcgaatacgcgatcgcagctttgaacattgagcatgttgtggtctgcggtcactcgcactgcggtgcgatgaaagggctgctcaagctcaatcagctgcaagaggacatgccgctggtctatgactggctgcagcatgcccaagccacccgccgcctagtcttggataactacagcggttatgagactgacgacttggtagagattctggtcgccgagaatgtgctgacgcagatcgagaaccttaagacctacccgatcgtgcgatcgcgccttttccaaggcaagctgcagatttttggctggatttatgaagttgaaagcggcgaggtcttgcagattagccgtaccagcagtgatgacacaggcattgatgaatgtccagtgcgtttgcccggcagccaggagaaagccattctcggtcgttgtgtcgtccccctgaccgaagaagtggccgttgctccaccagagccggagcctgtgatcgcggctgtggcggctccacccgccaactactccagtcgcggttggttggcccctgaacaacaacagcggatttatcgcggcaatgctagctag

SEQ ID NO: 58 - protein, 272 aa, Synechococcus elongatus
MRKLIEGLRHFRTSYYPSHRDLFEQFAKGQHPRVLFITCSDSRIDPNLITQSGMGELFVIRNAGNLIPPFGAANGGEGASIEYAIAALNIEHVVVCGHSHCGAMKGLLKLNQLQEDMPLVYDWLQHAQATRRLVLDNYSGYETDDLVEILVAENVLTQIENLKTYPIVRSRLFQGKLQIFGWIYEVESGEVLQISRTSSDDTGIDECPVRLPGSQEKAILGRCVVPLTEEVAVAPPEPEPVIAAVAAPPANYSSRGWLAPEQQQRIYRGNAS

SEQ ID NO: 59 - DNA, 486 bp, Synechococcus elongatus
atgcacctaccacctctggaacctccaattagtgatcgatattttgcttcaggtgaggttacaattgcagctgatgtagttatagcacctggggtattgcttattgcagaagctgacagtcggattgaaattgcatcaggagtttgtattggactcggcagtgtaattcatgcacgaggaggtgcaattataattcaagcaggcgctttactggcagctggcgtacttattgttggacaatcaattgttgggcggcaagcatgtcttggtgcatccactacccttgttaatacttctattgaggctggaggtgttacagcaccaggaagtttactttcagctgaaacacctcccacgactgctacagttagttcctcagagcctgctgggaggtctccccaatcctcagcaattgctcatcctaccaaagtatatggaaaagaacaatttttaaggatgcgacaaagtatgttccctgatcgataa

SEQ ID NO: 60 - protein, 161 aa, Synechococcus elongatus
MHLPPLEPPISDRYFASGEVTIAADVVIAPGVLLIAEADSRIEIASGVCIGLGSVIHARGGAIIIQAGALLAAGVLIVGQSIVGRQACLGASTTLVNTSIEAGGVTAPGSLLSAETPPTTATVSSSEPAGRSPQSSAIAHPTKVYGKEQFLRMRQSMFPDR

SEQ ID NO: 61 - protein, 472 aa, Synechococcus elongatus; identical to SEQ ID NO: 46

SEQ ID NO: 62 - protein, 111 aa, Synechococcus elongatus; identical to SEQ ID NO: 48

SEQ ID NO: 63 - protein, 470 aa, Prochlorococcus marinus
MSKKYDAGVKEYRDTYWTPDYVPLDTDLLACFKCTGQEGVPREEVAAAVAAESSTGTWSTVWSELLTDLEFYKGRCYRIEDVPGDKESFYAFIAYPLDLFEEGSITNVLTSLVGNVFGFKALRHLRLEDIRFPMAFIKTCGGPPNGIVVERDRLNKYGRPLLGCTIKPKLGLSGKNYGRVVYECLRGGLDLTKDDENINSQPFQRWRERFEFVAEAVKLAQQETGEVKGHYLNCTATTPEEMYERAEFAKELDMPIIMHDYITGGFTANTGLANWCRKNGMLLHIHRAMHAVIDRHPKHGIHFRVLAKCLRLSGGDQLHTGTVVGKLEGDRQTTLGYIDNLRESFVPEDRSRGNFFDQDWGSMPGVFAVASGGIHVWHMPALLAIFGDDSCLQFGGGTHGHPWGSAAGAAANRVALEACVKARNAGREIEKESRDILMEAAKHSPELAIALETWKEIKFEFDTVDKLDVQ

SEQ ID NO: 64 - protein, 113 aa, Prochlorococcus marinus
MPFQSTVGDYQTVATLETFGFLPPMTQDEIYDQIAYIIAQGWSPVIEHVHPSGSMQTYWSYWKLPFFGEKDLNMVVSELEACHRAYPDHHVRMVGYDAYTQSQGTAFVVFEGR

SEQ ID NO: 65 - protein, 473 aa, Halothiobacillus neapolitanus
MAVKKYSAGVKEYRQTYWMPEYTPLDSDILACFKITPQPGVDREEAAAAVAAESSTGTWTTVWTDLLTDMDYYKGRAYRIEDVPGDDAAFYAFIAYPIDLFEEGSVVNVFTSLVGNVFGFKAVRGLRLEDVRFPLAYVKTCGGPPHGIQVERDKMNKYGRPLLGCTIKPKLGLSAKNYGRAVYECLRGGLDFTKDDENINSQPFMRWRDRFLFVQDATETAEAQTGERKGHYLNVTAPTPEEMYKRAEFAKEIGAPIIMHDYITGGFTANTGLAKWCQDNGVLLHIHRAMHAVIDRNPNHGIHFRVLTKILRLSGGDHLHTGTVVGKLEGDRASTLGWIDLLRESFIPEDRSRGIFFDQDWGSMPGVFAVASGGIHVWHMPALVNIFGDDSVLQFGGGTLGHPWGNAAGAAANRVALEACVEARNQGRDIEKEGKEILTAAAQHSPELKIAMETWKEIKFEFDTVDKLDTQNR

SEQ ID NO: 66 - protein, 110 aa, Halothiobacillus neapolitanus
MAEMQDYKQSLKYETFSYLPPMNAERIRAQIKYAIAQGWSPGIEHVEVKNSMNQYWYMWKLPFFGEQNVDNVLAEIEACRSAYPTHQVKLVAYDNYAQSLGLAFVVYRGN

SEQ ID NO: 67 - protein, 486 aa, Rhodobacter sphaeroides
MDTKTTEIKGKERYKAGVLKYAQMGYWDGDYVPKDTDVLALFRITPQEGVDPVEAAAAVAGESSTATWTVVWTDRLTACDSYRAKAYRVEPVPGTPGQYFCYVAYDLILFEEGSIANLTASIIGNVFSFKPLKAARLEDMRFPVAYVKTYKGPPTGIVGERERLDKFGKPLLGATTKPKLGLSGKNYGRVVYEGLKGGLDFMKDDENINSQPFMHWRDRFLYVMEAVNLASAQTGEVKGHYLNITAGTMEEMYRRAEFAKSLGSVIVMVDLIIGYTAIQSISEWCRQNDMILHMHRAGHGTYTRQKNHGISFRVIAKWLRLAGVDHLHCGTAVGKLEGDPLTVQGYYNVCREPFNTVDLPRGIFFEQDWADLRKVMPVASGGIHAGQMHQLLSLFGDDVVLQFGGGTIGHPMGIQAGATANRVALEAMVLARNEGRNIDVEGPEILRAAAKWCKPLEAALDTWGNITFNYTSTDTSDFVPTASVAM

SEQ ID NO: 68 - DNA, 61 bp, Artificial Sequence
  Synthetic Construct
gtcaacagatctcaagaaggagatatacccatggttagtaaaggtgaagaattgtttactg

SEQ ID NO: 69 - DNA, 53 bp, Artificial Sequence
  Synthetic Construct
tgaacccccacttccaccagaacctcccttgtacaactcgtccattcctaaag

SEQ ID NO: 70 - DNA, 43 bp, Artificial Sequence
  Synthetic Construct
ctggtggaagtgggggttcatctgcttataacggacaaggtcg

SEQ ID NO: 71 - DNA, 44 bp, Artificial Sequence
  Synthetic Construct
agatgcggccgcacgcgtttacggcttctgaatcaacaactcag

SEQ ID NO: 72 - DNA, 51 bp, Artificial Sequence
  Synthetic Construct
gaaattaatacgactcactatagggttacttatccaaaacgtccactgctg

SEQ ID NO: 73 - DNA, 24 bp, Artificial Sequence
  Synthetic Construct
atcatattcactctggtaccgtag

SEQ ID NO: 74 - DNA, 49 bp, Artificial Sequence
  Synthetic Construct
gaaattaatacgactcactatagggtttgccaactaccttagtgatctc

SEQ ID NO: 75 - DNA, 22 bp, Artificial Sequence
  Synthetic Construct
atggctcgtgaagcggttatcg

SEQ ID NO: 76 - DNA, 53 bp, Artificial Sequence
  Synthetic Construct
gaaattaatacgactcactatagggaagcttatccattgtctcaaattcaaac

SEQ ID NO: 77 - DNA, 27 bp, Artificial Sequence
  Synthetic Construct
gtatgccaatcataatgcatgattttc

SEQ ID NO: 78 - DNA, 50 bp, Artificial Sequence
  Synthetic Construct
gaaattaatacgactcactatagggatatcttccaggtcgatgcacaatg

SEQ ID NO: 79 - DNA, 35 bp, Artificial Sequence
  Synthetic Construct
ggatccatgagtatgaaaaccttgccaaaagaacg

SEQ ID NO: 80 - DNA, 48 bp, Artificial Sequence
  Synthetic Construct
gaaattaatacgactcactatagggtcaatccgcatgggaggcattag

SEQ ID NO: 81 - DNA, 19 bp, Artificial Sequence
  Synthetic Construct
cgcatcagtcgcgatacag

SEQ ID NO: 82 - DNA, 49 bp, Artificial Sequence
  Synthetic Construct
gaaattaatacgactcactatagggcggcttttgaatcaacagttcagc

SEQ ID NO: 83 - DNA, 21 bp, Artificial Sequence
  Synthetic Construct
tcctgcgcaccgattcaaagt

SEQ ID NO: 84 - DNA, 46 bp, Artificial Sequence
  Synthetic Construct
gaaattaatacgactcactatagggcggcttctgaatcaacaactc

SEQ ID NO: 85 - DNA, 24 bp, Artificial Sequence
  Synthetic Construct
acctgatggttcggttcctgaatc

SEQ ID NO: 86 - DNA, 6119 bp, Artificial Sequence
  Synthetic Construct
cactatctcgaccttgaactaccagagcgttataaatattcggcatcttgcccgggggaaaggctacatctagtaccggaccgatgatttggacgacacgccccgggttttttttttcaagcgtggaaaccccagaaccagaagtagtaggattgattctcataataataaaataaataaatatgtcgaaatgtttttgcaaaaattatcgaattcaaaataaatgtccgctagcacgtcgatcggttaattcaataaaatgggaattagcactcgatttcgttggcaccatgcaattgaaccgattcaattgtttacttattcactgagactgagtgaatttgcaagcccacccaacctattttaattttaaaatctcaagtggatgaatcagaatcttgagaaagtctttcatttgtctatcattatagacaatcccatccatattatctattctatggaattcgaacctgaactttattttctatttctattacgattcattatttgtatctaattggctcctcttcttatttatttttgatttcaatttcagcatatcgatttatgcctagcctattcttttctttgtgtttttctttcttttttatacctttcatagattcatagaggaattccgtatattttcacatctaggatttacatatacaacatataccactgtcaagggggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataatattagtattagttggaaattttgtgaaagattcctgtgaaaagtttcattaacacggaattcgtgtcgagtagaccttgttgttgtgagaattcttaattcatgagttgtagggagggatttatgcctaaaacccaaagtgctgctggatataaagcaggagttaaagattataaacttacctattatactccagattatactccaaaagataccgatttacttgctgcatttcgattcagtcctcaaccaggagtaccagcagatgaagctggtgctgcaattgcagcagaaagttcaacaggaacttggactaccgtttggacagatcttctaaccgatatggatagatataaagggaaatgttatcatattgaaccagtacaaggagaagagaattcctattttgcttttattgcatatcctcttgatttgtttgaagaaggatcagttactaacattctaactagtatcgttggaaatgtatttggattcaaagctatacgatcactacgtttggaagatatacgtttcccagttgctttggttaaaactttccaagggcctccacatggaattcaagttgaaagagatttattaaacaagtatgggcgaccaatgcttggatgtacaattaagcctaaattagggctatctgctaaaaactatggacgtgctgtatatgagtgtttaagaggaggattagattttactaaagatgatgaaaatattaattcacaaccttttcaacgatggcgagatagatttctttttgttgccgatgccattcataaatcacaagccgagactggagaaattaagggacattatctaaatgtaaccgccccaacatgtgaagaaatgatgaagcgagctgaatttgctaaagaattgggtatgccaatcataatgcatgattttctaactgctggattcaccgccaatactactttagctaagtggtgtcgtgataatggtgtattacttcatatacatcgagcaatgcatgctgtaatagatagacaacgaaaccatggtattcattttcgtgttttagcaaaatgtcttcgattgagtggaggggatcatttgcattctgggactgttgtagggaaattggaaggagataaagcctcaactcttggatttgtagatctaatgcgagaagatcatatagaggcagatagaagtagaggtgtatttttcacccaagattgggctagtatgcctggggttcttcctgtagctagtggaggaattcatgtttggcacatgccagcactagtagaaatcttcggagatgattcagttttacaatttggtggaggaactctaggtcatccatggggaaatgcaccaggtgcaacagcaaatcgtgttgctttagaagcatgtgtacaagctcgtaatgagggtcgagatttatatagagaagggggagatatacttagagaggctggaaaatggtctccagaattggcagctgcccttgatctatggaaagaaataaagtttgaatttgagacaatggataagctttaacgcgcgtcgcgcgtcgcgcgcgttcaatttattcaattgtaaaataaacgacgtgggtatctagggaggtatctagggagtagtcatttccaaatgaattctccctagatacatatctaattctaattaatttaattaattaattaaaatgggttaagatcatttatttacaacggaatggtatacaaagtcaacagatctcaagaaggagatatacccatgagtatgaaaaccttgccaaaagaacgaagatttgaaaccttcagttatttacctcctctttctgatcgtcaaattgctgctcaaatcgaatatatgatagaacaaggttttcatccattgatagaatttaatgaacattcaaatccagaagaattctattggaccatgtggaaactacctttgtttgattgtaagtctccacaacaagtattggatgaggtacgagaatgtcgttccgagtatggtgattgttatattagagttgcaggatttgataacatcaaacaatgtcaaaccgttagtttcattgtgcatcgacctggaagatattaaacgcgcgttcgttagtgttagtctagatctagtttagtaaaaaacgagcaatataagccttctttaaataagaaagagggcttatattactcgtttttttctataaaaatgagcaaatttttatagagtatcatatttaagatcatttatttacaacggaatggtatacaaagtcaacagatctcaagaaggagatatacccatggcgtcaacgcagagggcgaagccgatggagatgccccgcatcagtcgcgatacagcccgcatgttggtcaattacctgacctatcaagcggtctgtgtgattcgggatcaattggctgagacgaatccggccggtgcataccggctgcaggttttctcggctgagttctcctttcaggatggagaagcttacctagcagctctactcaaccacgatcgcgaattgggcctgcgggtgatgacagtacgggaacatttggccgagcatattctcgactacctgccggagatgacgatcgctcagatccaggaggcgaatattaatcatcgccgtgctttgcttgaacggctgacgggtcttggggcagagcctagcttgccggagaccgaggtgagcgatcgccccagtgactcagccactcctgatgatgcttctaatgcctcccatgcggattgaacgcgcgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaagggccgcctgccctgcagataacttcgtataatgtatgctatacgaagttatcccgggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataatcaaccgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtataacttcgtataatgtatgctatacgaagttatcctgcagatgcaggtcgaccatatgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagaaaactaaatcaaaatctaagactcaaatctttctattgttgtcttggatccacaattaatcctacggatccttaggattggtatattcttttctatcctgtagtttgtagtttccctgaatcaagccaagtatcacacctctttctacccatcctgtatattgtcccctttgttccgtgttgaaatagaaccttaatttattacttatttttttattaaattttagatttgttagtgattagatattagtattagacgagattttacgaaacaattatttttttatttctttataggagaggacaaatctcttttttcgatgcgaatttgacacgacataggagaagccgccctttattaaaaattatattattttaaataatataaagggggttccaacatattaatatatagtgaagtgttcccccagattcagaactttttttcaatactcacaatccttattagttaataatcctagtgattggatttctatgcttagtctgataggaaataagatattcaaataaataattttatagcgaatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcctattgaaaataccaatgaagatccaaatcgaaaagtgaaaaacattcatagttggaggaatcgtgacaattctagttgcagtaatgttgattattt

SEQ ID NO: 87 - DNA, 6665 bp, Artificial Sequence
  Synthetic Construct
cactatctcgaccttgaactaccagagcgttataaatattcggcatcttgcccgggggaaaggctacatctagtaccggaccgatgatttggacgacacgccccgggttttttttttcaagcgtggaaaccccagaaccagaagtagtaggattgattctcataataataaaataaataaatatgtcgaaatgtttttgcaaaaattatcgaattcaaaataaatgtccgctagcacgtcgatcggttaattcaataaaatgggaattagcactcgatttcgttggcaccatgcaattgaaccgattcaattgtttacttattcactgagactgagtgaatttgcaagcccacccaacctattttaattttaaaatctcaagtggatgaatcagaatcttgagaaagtctttcatttgtctatcattatagacaatcccatccatattatctattctatggaattcgaacctgaactttattttctatttctattacgattcattatttgtatctaattggctcctcttcttatttatttttgatttcaatttcagcatatcgatttatgcctagcctattcttttctttgtgtttttctttcttttttatacctttcatagattcatagaggaattccgtatattttcacatctaggatttacatatacaacatataccactgtcaagggggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataatattagtattagttggaaattttgtgaaagattcctgtgaaaagtttcattaacacggaattcgtgtcgagtagaccttgttgttgtgagaattcttaattcatgagttgtagggagggatttatgcctaaaacccaaagtgctgctggatataaagcaggagttaaagattataaacttacctattatactccagattatactccaaaagataccgatttacttgctgcatttcgattcagtcctcaaccaggagtaccagcagatgaagctggtgctgcaattgcagcagaaagttcaacaggaacttggactaccgtttggacagatcttctaaccgatatggatagatataaagggaaatgttatcatattgaaccagtacaaggagaagagaattcctattttgcttttattgcatatcctcttgatttgtttgaagaaggatcagttactaacattctaactagtatcgttggaaatgtatttggattcaaagctatacgatcactacgtttggaagatatacgtttcccagttgctttggttaaaactttccaagggcctccacatggaattcaagttgaaagagatttattaaacaagtatgggcgaccaatgcttggatgtacaattaagcctaaattagggctatctgctaaaaactatggacgtgctgtatatgagtgtttaagaggaggattagattttactaaagatgatgaaaatattaattcacaaccttttcaacgatggcgagatagatttctttttgttgccgatgccattcataaatcacaagccgagactggagaaattaagggacattatctaaatgtaaccgccccaacatgtgaagaaatgatgaagcgagctgaatttgctaaagaattgggtatgccaatcataatgcatgattttctaactgctggattcaccgccaatactactttagctaagtggtgtcgtgataatggtgtattacttcatatacatcgagcaatgcatgctgtaatagatagacaacgaaaccatggtattcattttcgtgttttagcaaaatgtcttcgattgagtggaggggatcatttgcattctgggactgttgtagggaaattggaaggagataaagcctcaactcttggatttgtagatctaatgcgagaagatcatatagaggcagatagaagtagaggtgtatttttcacccaagattgggctagtatgcctggggttcttcctgtagctagtggaggaattcatgtttggcacatgccagcactagtagaaatcttcggagatgattcagttttacaatttggtggaggaactctaggtcatccatggggaaatgcaccaggtgcaacagcaaatcgtgttgctttagaagcatgtgtacaagctcgtaatgagggtcgagatttatatagagaagggggagatatacttagagaggctggaaaatggtctccagaattggcagctgcccttgatctatggaaagaaataaagtttgaatttgagacaatggataagctttaacgcgcgtcgcgcgtcgcgcgcgagtcttactaaaacgaaatgaaattaatgaaatataaaaaagaggatgtgaaagactcgtatataacgtgatataaaattttatctactcttctctttcacttccatcatatttattttgtaagatcatttatttacaacggaatggtatacaaagtcaacagatctcaagaaggagatatacccatgagtatgaaaaccttgccaaaagaacgaagatttgaaaccttcagttatttacctcctctttctgatcgtcaaattgctgctcaaatcgaatatatgatagaacaaggttttcatccattgatagaatttaatgaacattcaaatccagaagaattctattggaccatgtggaaactacctttgtttgattgtaagtctccacaacaagtattggatgaggtacgagaatgtcgttccgagtatggtgattgttatattagagttgcaggatttgataacatcaaacaatgtcaaaccgttagtttcattgtgcatcgacctggaagatattaaacgcgcgttcgttagtgttagtctagatctagtttagtaaaaaacgagcaatataagccttctttaaataagaaagagggcttatattactcgtttttttctataaaaatgagcaaatttttatagagtatcatatttaagatcatttatttacaacggaatggtatacaaagtcaacagatctcaagaaggagatatacatatgggagaaggagatatacatatgggagaaggagatatacccatgagcgcttataacggccaaggccgactcagttccgaagtcatcacccaagtccggagtttgctgaaccagggctatcggattgggacggaacatgcggacaagcgccgcttccggactagctcttggcagccctgcgcgccgattcaaagcacgaacgagcgccaggtcttgagcgaactggaaaattgtctgagcgaacacgaaggtgaatacgttcgcttgctcggcatcgataccaatactcgcagccgtgtttttgaagccctgattcaacggcccgatggttcggttcctgaatcgctggggagccaaccggtggcagtcgcttccggtggtggccgtcagagcagctatgccagcgtcagcggcaacctctcagcagaagtggtcaataaagtccgcaacctcttagcccaaggctatcggattgggacggaacatgcagacaagcgccgctttcggactagctcttggcagtcctgcgcaccgattcaaagttcgaatgagcgccaggttctggctgaactggaaaactgtctgagcgagcacgaaggtgagtacgttcgcctgctgggcatcgacactgctagccgcagtcgtgtttttgaagccctgatccaagatccccaaggaccggtgggttccgccaaagctgccgccgcacctgtgagttcggcaacgcccagcagccacagctacacctcaaatggatcgagttcgagcgatgtcgctggacaggttcggggtctgctagcccaaggctaccggatcagtgcggaagtcgccgataagcgtcgcttccaaaccagctcttggcagagtttgccggctctgagtggccagagcgaagcaactgtcttgcctgctttggagtcaattctgcaagagcacaagggtaagtatgtgcgcctgattgggattgaccctgcggctcgtcgtcgcgtggctgaactgttgattcaaaagccgtaaacgcgcgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaagggccgcctgccctgcagataacttcgtataatgtatgctatacgaagttatcccgggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataatcaaccgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtataacttcgtataatgtatgctatacgaagttatcctgcagatgcaggtcgaccatatgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagaaaactaaatcaaaatctaagactcaaatctttctattgttgtcttggatccacaattaatcctacggatccttaggattggtatattcttttctatcctgtagtttgtagtttccctgaatcaagccaagtatcacacctctttctacccatcctgtatattgtcccctttgttccgtgttgaaatagaaccttaatttattacttatttttttattaaattttagatttgttagtgattagatattagtattagacgagattttacgaaacaattatttttttatttctttataggagaggacaaatctcttttttcgatgcgaatttgacacgacataggagaagccgccctttattaaaaattatattattttaaataatataaagggggttccaacatattaatatatagtgaagtgttcccccagattcagaactttttttcaatactcacaatccttattagttaataatcctagtgattggatttctatgcttagtctgataggaaataagatattcaaataaataattttatagcgaatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcctattgaaaataccaatgaagatccaaatcgaaaagtgaaaaacattcatagttggaggaatcgtgacaattctagttgcagtaatgttgattattt

SEQ ID NO: 88 - DNA, 5439 bp, Artificial Sequence
  Synthetic Construct
cactatctcgaccttgaactaccagagcgttataaatattcggcatcttgcccgggggaaaggctacatctagtaccggaccgatgatttggacgacacgccccgggttttttttttcaagcgtggaaaccccagaaccagaagtagtaggattgattctcataataataaaataaataaatatgtcgaaatgtttttgcaaaaattatcgaattcaaaataaatgtccgctagcacgtcgatcggttaattcaataaaatgggaattagcactcgatttcgttggcaccatgcaattgaaccgattcaattgtttacttattcactgagactgagtgaatttgcaagcccacccaacctattttaattttaaaatctcaagtggatgaatcagaatcttgagaaagtctttcatttgtctatcattatagacaatcccatccatattatctattctatggaattcgaacctgaactttattttctatttctattacgattcattatttgtatctaattggctcctcttcttatttatttttgatttcaatttcagcatatcgatttatgcctagcctattcttttctttgtgtttttctttcttttttatacctttcatagattcatagaggaattccgtatattttcacatctaggatttacatatacaacatataccactgtcaagggggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataatattagtattagttggaaattttgtgaaagattcctgtgaaaagtttcattaacacggaattcgtgtcgagtagaccttgttgttgtgagaattcttaattcatgagttgtagggagggatttatgcctaaaacccaaagtgctgctggatataaagcaggagttaaagattataaacttacctattatactccagattatactccaaaagataccgatttacttgctgcatttcgattcagtcctcaaccaggagtaccagcagatgaagctggtgctgcaattgcagcagaaagttcaacaggaacttggactaccgtttggacagatcttctaaccgatatggatagatataaagggaaatgttatcatattgaaccagtacaaggagaagagaattcctattttgcttttattgcatatcctcttgatttgtttgaagaaggatcagttactaacattctaactagtatcgttggaaatgtatttggattcaaagctatacgatcactacgtttggaagatatacgtttcccagttgctttggttaaaactttccaagggcctccacatggaattcaagttgaaagagatttattaaacaagtatgggcgaccaatgcttggatgtacaattaagcctaaattagggctatctgctaaaaactatggacgtgctgtatatgagtgtttaagaggaggattagattttactaaagatgatgaaaatattaattcacaaccttttcaacgatggcgagatagatttctttttgttgccgatgccattcataaatcacaagccgagactggagaaattaagggacattatctaaatgtaaccgccccaacatgtgaagaaatgatgaagcgagctgaatttgctaaagaattgggtatgccaatcataatgcatgattttctaactgctggattcaccgccaatactactttagctaagtggtgtcgtgataatggtgtattacttcatatacatcgagcaatgcatgctgtaatagatagacaacgaaaccatggtattcattttcgtgttttagcaaaatgtcttcgattgagtggaggggatcatttgcattctgggactgttgtagggaaattggaaggagataaagcctcaactcttggatttgtagatctaatgcgagaagatcatatagaggcagatagaagtagaggtgtatttttcacccaagattgggctagtatgcctggggttcttcctgtagctagtggaggaattcatgtttggcacatgccagcactagtagaaatcttcggagatgattcagttttacaatttggtggaggaactctaggtcatccatggggaaatgcaccaggtgcaacagcaaatcgtgttgctttagaagcatgtgtacaagctcgtaatgagggtcgagatttatatagagaagggggagatatacttagagaggctggaaaatggtctccagaattggcagctgcccttgatctatggaaagaaataaagtttgaatttgagacaatggataagctttaacgcgcgtcgcgcgcgagtcttactaaaacgaaatgaaattaatgaaatataaaaaagaggatgtgaaagactcgtatataacgtgatataaaattttatctactcttctctttcacttccatcatatttattttgtaagatcatttatttacaacggaatggtatacaaagtcaacagatctcaagaaggagatatacccatgagtatgaaaaccttgccaaaagaacgaagatttgaaaccttcagttatttacctcctctttctgatcgtcaaattgctgctcaaatcgaatatatgatagaacaaggttttcatccattgatagaatttaatgaacattcaaatccagaagaattctattggaccatgtggaaactacctttgtttgattgtaagtctccacaacaagtattggatgaggtacgagaatgtcgttccgagtatggtgattgttatattagagttgcaggatttgataacatcaaacaatgtcaaaccgttagtttcattgtgcatcgacctggaagatattaaacgcgcgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaagggccgcctgccctgcagataacttcgtataatgtatgctatacgaagttatcccgggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataatcaaccgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtataacttcgtataatgtatgctatacgaagttatcctgcagatgcaggtcgaccatatgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagaaaactaaatcaaaatctaagactcaaatctttctattgttgtcttggatccacaattaatcctacggatccttaggattggtatattcttttctatcctgtagtttgtagtttccctgaatcaagccaagtatcacacctctttctacccatcctgtatattgtcccctttgttccgtgttgaaatagaaccttaatttattacttatttttttattaaattttagatttgttagtgattagatattagtattagacgagattttacgaaacaattatttttttatttctttataggagaggacaaatctcttttttcgatgcgaatttgacacgacataggagaagccgccctttattaaaaattatattattttaaataatataaagggggttccaacatattaatatatagtgaagtgttcccccagattcagaactttttttcaatactcacaatccttattagttaataatcctagtgattggatttctatgcttagtctgataggaaataagatattcaaataaataattttatagcgaatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcctattgaaaataccaatgaagatccaaatcgaaaagtgaaaaacattcatagttggaggaatcgtgacaattctagttgcagtaatgttgattattt

SEQ ID NO: 89 - DNA, 7408 bp, Artificial Sequence
  Synthetic Construct
cactatctcgaccttgaactaccagagcgttataaatattcggcatcttgcccgggggaaaggctacatctagtaccggaccgatgatttggacgacacgccccgggttttttttttcaagcgtggaaaccccagaaccagaagtagtaggattgattctcataataataaaataaataaatatgtcgaaatgtttttgcaaaaattatcgaattcaaaataaatgtccgctagcacgtcgatcggttaattcaataaaatgggaattagcactcgatttcgttggcaccatgcaattgaaccgattcaattgtttacttattcactgagactgagtgaatttgcaagcccacccaacctattttaattttaaaatctcaagtggatgaatcagaatcttgagaaagtctttcatttgtctatcattatagacaatcccatccatattatctattctatggaattcgaacctgaactttattttctatttctattacgattcattatttgtatctaattggctcctcttcttatttatttttgatttcaatttcagcatatcgatttatgcctagcctattcttttctttgtgtttttctttcttttttatacctttcatagattcatagaggaattccgtatattttcacatctaggatttacatatacaacatataccactgtcaagggggaagttcttattatttaggttagtcaggtatttccatttcaaaaaaaaaaaaagtaaaaaagaaaaattgggttgcgctatatatatgaaagagtatacaataatgatgtatttggcaaatcaaataccatggtctaataatcaaacattctgattagttgataatattagtattagttggaaattttgtgaaagattcctgtgaaaagtttcattaacacggaattcgtgtcgagtagaccttgttgttgtgagaattcttaattcatgagttgtagggagggatttatgcctaaaacccaaagtgctgctggatataaagcaggagttaaagattataaacttacctattatactccagattatactccaaaagataccgatttacttgctgcatttcgattcagtcctcaaccaggagtaccagcagatgaagctggtgctgcaattgcagcagaaagttcaacaggaacttggactaccgtttggacagatcttctaaccgatatggatagatataaagggaaatgttatcatattgaaccagtacaaggagaagagaattcctattttgcttttattgcatatcctcttgatttgtttgaagaaggatcagttactaacattctaactagtatcgttggaaatgtatttggattcaaagctatacgatcactacgtttggaagatatacgtttcccagttgctttggttaaaactttccaagggcctccacatggaattcaagttgaaagagatttattaaacaagtatgggcgaccaatgcttggatgtacaattaagcctaaattagggctatctgctaaaaactatggacgtgctgtatatgagtgtttaagaggaggattagattttactaaagatgatgaaaatattaattcacaaccttttcaacgatggcgagatagatttctttttgttgccgatgccattcataaatcacaagccgagactggagaaattaagggacattatctaaatgtaaccgccccaacatgtgaagaaatgatgaagcgagctgaatttgctaaagaattgggtatgccaatcataatgcatgattttctaactgctggattcaccgccaatactactttagctaagtggtgtcgtgataatggtgtattacttcatatacatcgagcaatgcatgctgtaatagatagacaacgaaaccatggtattcattttcgtgttttagcaaaatgtcttcgattgagtggaggggatcatttgcattctgggactgttgtagggaaattggaaggagataaagcctcaactcttggatttgtagatctaatgcgagaagatcatatagaggcagatagaagtagaggtgtatttttcacccaagattgggctagtatgcctggggttcttcctgtagctagtggaggaattcatgtttggcacatgccagcactagtagaaatcttcggagatgattcagttttacaatttggtggaggaactctaggtcatccatggggaaatgcaccaggtgcaacagcaaatcgtgttgctttagaagcatgtgtacaagctcgtaatgagggtcgagatttatatagagaagggggagatatacttagagaggctggaaaatggtctccagaattggcagctgcccttgatctatggaaagaaataaagtttgaatttgagacaatggataagctttaacgcgcgtcgcgcgtcgcgcgcgttcaatttattcaattgtaaaataaacgacgtgggtatctagggaggtatctagggagtagtcatttccaaatgaattctccctagatacatatctaattctaattaatttaattaattaattaaaatgggttaagatcatttatttacaacggaatggtatacaaagtcaacagatctcaagaaggagatatacccatgagtatgaaaaccttgccaaaagaacgaagatttgaaaccttcagttatttacctcctctttctgatcgtcaaattgctgctcaaatcgaatatatgatagaacaaggttttcatccattgatagaatttaatgaacattcaaatccagaagaattctattggaccatgtggaaactacctttgtttgattgtaagtctccacaacaagtattggatgaggtacgagaatgtcgttccgagtatggtgattgttatattagagttgcaggatttgataacatcaaacaatgtcaaaccgttagtttcattgtgcatcgacctggaagatattaaacgcgcgagtcttactaaaacgaaatgaaattaatgaaatataaaaaagaggatgtgaaagactcgtatataacgtgatataaaattttatctactcttctctttcacttccatcatatttattttgtaagatcatttatttacaacggaatggtatacaaagtcaacagatctcaagaaggagatatacatatgggagaaggagatatacatatgggagaaggagatatacccatggttagtaaaggtgaagaattgtttactggagtagttcctattcttgtagagttagatggagatgtaaatggacataaattctctgtatctggtgaaggtgaaggagatgctacttatggtaaattgaccttgaagtttatctgtactactggaaaacttccagtaccatggcctacacttgtaactacatttggatatggtgtacaatgttttgcaagatatcctgatcacatgaaacagcatgatttctttaaatctgctatgcctgaaggatatgttcaagaacgaaccatctttttcaaagacgatggtaactacaaaactagagctgaggttaagtttgaaggagatactttagttaatcgaattgaattgaaaggaatagatttcaaagaggacggtaatattcttggacataaacttgaatataattacaatagtcacaatgtatatattatggctgataaacagaagaatggaatcaaagttaacttcaaaattcgacataacatagaagatggatctgtacaattagctgaccattatcaacagaatactccaattggagatggtcctgtattacttcctgataatcactatcttagttatcaatctgcattaagtaaagatcctaatgaaaaacgtgatcacatggtattacttgaatttgtaactgctgctgggattactttaggaatggacgagttgtacaagggaggttctggtggaagtgggggttcatctgcttataacggacaaggtcgattaagttctgaagtaattactcaagttcgaagtttgttaaaccaaggatatcgaattggaactgaacatgctgataagagacgatttagaactagttcttggcaaccttgtgctcctattcaatctactaatgagagacaggtattgtctgaacttgaaaattgtctttctgaacatgaaggtgaatacgttcgattgttaggaattgataccaatactagatctcgtgtttttgaagctttaattcaacgacctgatggttcggttcctgaatcgttaggatctcaacctgtggcagtagcttcaggtggaggtcgacaatcatcttatgcaagtgtatctggaaatttatctgctgaagtagttaataaagtacgtaatctattagctcaaggatatcgaattggtacagaacacgcagacaaaagacgatttcgtacttcttcatggcagtcatgcgcaccaatccagagttctaacgagcgtcaagttcttgctgagcttgaaaactgcttaagtgagcatgagggagagtacgttagattacttggtatcgatactgcttctagaagtcgtgttttcgaagcacttatacaagatccacaaggacctgtaggttctgctaaagctgcagccgctcctgtatcttcagctactccaagttctcatagttatacttctaatggatctagttcgagcgatgtcgctggacaggttcgaggtcttctagcacagggttaccgtataagtgctgaagtagctgataagcgtagattccaaacaagttcttggcaaagtttacctgctcttagtggacagtctgaagcaactgtattgcctgctttggagtcaattcttcaagaacacaaaggtaagtatgtacgtcttattgggattgaccctgcagctcgtcgtcgagtagctgagttgttgattcagaagccgtaaacgcgcgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaagggccgcctgccctgcagataacttcgtataatgtatgctatacgaagttatcccgggcaacccactagcatatcgaaattctaattttctgtagagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagattttaccatggctcgtgaagcggttatcgccgaagtatcaactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcactaaggtagttggcaaataatcaaccgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtataacttcgtataatgtatgctatacgaagttatcctgcagatgcaggtcgaccatatgaaacagtagacattagcagataaattagcaggaaataaagaaggataaggagaaagaactcaagtaattatccttcgttctcttaattgaattgcaattaaactcggcccaatcttttactaaaaggattgagccgaatacaacaaagattctattgcatatattttgactaagtatatacttacctagatatacaagatttgaaatacaaaatctagaaaactaaatcaaaatctaagactcaaatctttctattgttgtcttggatccacaattaatcctacggatccttaggattggtatattcttttctatcctgtagtttgtagtttccctgaatcaagccaagtatcacacctctttctacccatcctgtatattgtcccctttgttccgtgttgaaatagaaccttaatttattacttatttttttattaaattttagatttgttagtgattagatattagtattagacgagattttacgaaacaattatttttttatttctttataggagaggacaaatctcttttttcgatgcgaatttgacacgacataggagaagccgccctttattaaaaattatattattttaaataatataaagggggttccaacatattaatatatagtgaagtgttcccccagattcagaactttttttcaatactcacaatccttattagttaataatcctagtgattggatttctatgcttagtctgataggaaataagatattcaaataaataattttatagcgaatgactattcatctattgtattttcatgcaaatagggggcaagaaaactctatggaaagatggtggtttaattcgatgttgtttaagaaggagttcgaacgcaggtgtgggctaaataaatcaatgggcagtcttggtcctattgaaaataccaatgaagatccaaatcgaaaagtgaaaaacattcatagttggaggaatcgtgacaattctagttgcagtaatgttgattattt

SEQ ID NO: 90 - DNA, 1449 bp, Limonium gibertii
atgagttgtagggagggacttatgtcaccacaaacagagactaaatcttttgttggattcaaagctggtgttaaagattacaaattgacttattatactcctgaatatgaaaccctagatactgatatcttggcagcatttcgagtaactcctcaacctggagttccaccagaggaagcaggggctgcagtagccgccgaatcttctactggtacatggacaactgtgtggaccgatggacttaccaaccttgatcgttacaaaggacgatgctaccacatcgagcctgttgctggagaagaaagtcaatttattgcttatgtagcttacccattagacctttttgaagaaggttctgtgactaatatgtttacttccattgtgggtaatgtatttgggttcaaagctctacgtgctctacgtttggaagatttgagaatccctcctgcttattcaaaaactttccaaggcccgcctcacggtatccaagttgaaagagataaattgaacaaatatggtcgtcccctgttgggatgtactattaaacctaaattggggttgtccgctaagaactacggccgagctgtttatgaatgtcttcgcggtggacttgattttaccaaagatgatgaaaacgtgaactcccaaccatttatgcgttggagagaccgtttcttattttgtaccgaagctatttataaagcacaggctgaaacaggtgaagtcaaaggacattacttgaatgctactgcagctacatccgaagaaatgataaaaagagctgcgtgtgctagagaattgggagttcctatcgtaatgcacgactatttaacagggggattcacttcaaatactagtttagctcattattgccgcgataatggcctacttcttcacatccaccgtgcaatgcacgcagttattgatagacagaaaaatcacggtatgcacttccgtgtactagctaaagccctacgtatgtctggtggagaccatattcatgctggtactgtagtaggtaaacttgaaggagaaagagagatcactttagggtttgttgatttactacgtgatgattatattgaaaaagaccgatctcgcggtatttatttcactcaagattgggtttccatgccgggtgttatacctgttgcttcgggcggtattcacgtttggcatatgcccgctctaaccgagatctttggagatgattccgtactgcaattcggtggtggaactttaggccacccttggggaaatgcaccaggtgctgtagcgaatcgagtagctctagaagcctgtgtacaagctcgtaatgagggacgtgatcttgctcgtgagggtaacgaaattatccgtcaagctgctacatggagtcctgaactagctgccgcttgtgaagtatggaaggaaatcaaatttgaattcgccgcaatggatactttgtaa

SEQ ID NO: 91 - protein, 482 aa, Limonium gibertii
MSCREGLMSPQTETKSFVGFKAGVKDYKLTYYTPEYETLDTDILAAFRVTPQPGVPPEEAGAAVAAESSTGTWTTVWTDGLTNLDRYKGRCYHIEPVAGEESQFIAYVAYPLDLFEEGSVTNMFTSIVGNVFGFKALRALRLEDLRIPPAYSKTFQGPPHGIQVERDKLNKYGRPLLGCTIKPKLGLSAKNYGRAVYECLRGGLDFTKDDENVNSQPFMRWRDRFLFCTEAIYKAQAETGEVKGHYLNATAATSEEMIKRAACARELGVPIVMHDYLTGGFTSNTSLAHYCRDNGLLLHIHRAMHAVIDRQKNHGMHFRVLAKALRMSGGDHIHAGTVVGKLEGEREITLGFVDLLRDDYIEKDRSRGIYFTQDWVSMPGVIPVASGGIHVWHMPALTEIFGDDSVLQFGGGTLGHPWGNAPGAVANRVALEACVQARNEGRDLAREGNEIIRQAATWSPELAAACEVWKEIKFEFAAMDTL

SEQ ID NO: 92 - DNA, 462 bp, Limonium gibertii
atgaccatgattacgccaagctcagaattaaccctcactaaagggactagtcctgcaggtttaaacgaattcgcccttggtggcagagtccgatgcatgcaggtatggccaccagagggtttgaagaagttcgagaccttgtcataccttccccctctagaccgtgaaggtctagccaacgagatctcttaccttatgagaatgggatgggttccctgcctggaattcgaagtcggcgaggcctacatccaccgtgagtaccacaacctcccaggatactatgacggacgctactggacaatgtggaagcttcccatgtacggatgcactgacccagctcaggtcttgaaggaagtcgacgagtgctctcagctttacccacacgcccacgtcaggatcctcggattcgacaacaagcgtcaagtgcagtgcatcagtttcatcgcctacaagccaccataa

SEQ ID NO: 93 - protein, 153 aa, Limonium gibertii
MTMITPSSELTLTKGTSPAGLNEFALGGRVRCMQVWPPEGLKKFETLSYLPPLDREGLANEISYLMRMGWVPCLEFEVGEAYIHREYHNLPGYYDGRYWTMWKLPMYGCTDPAQVLKEVDECSQLYPHAHVRILGFDNKRQVQCISFIAYKPP

(19) United States
(12) Patent Application Publication
(10) Pub. No.: US 2018/0298401 A1
(43) Pub. Date: Oct. 18, 2018

(54) ENGINEERING PHOTOSYNTHESIS

(71) Applicants: Cornell University, Ithaca, NY (US); Rothamsted Research Ltd., Hertfordshire (GB)

(72) Inventors: Maureen HANSON, Ithaca, NY (US); Myat LIN, Ithaca, NY (US); Martin Afan John PARRY, Steppingley, Bedford (GB)

(73) Assignees: Cornell University, Ithaca, NY (US); Rothamsted Research Ltd., Hertfordshire (GB)

(21) Appl. No.: 15/525,773

(22) PCT Filed: Nov. 12, 2015
(86) PCT No.: PCT/US15/60389

§ 371 Date: May 10, 2017

Related U.S. Application Data
(60) Provisional application No. 62/078,787, filed on Nov. 12, 2014.

(51) Int. Cl. C12N 15/82; C12N 9/88

(52) U.S. Cl. CPC C12N 15/8269; C12Y 401/01039; C12N 9/88

(57) ABSTRACT

Disclosed are plants including a cyanobacterial ribulose-1,5,-bisphosphate carboxylase/oxygenase (Rubisco) which can assemble and fix carbon without an interacting protein.

### CROSS-REFERENCE TO RELATED APPLICATIONS

This application claims benefit of U.S. Provisional Application No. 62/078,787, filed Nov. 12, 2014, which is hereby incorporated by reference in its entirety.

### STATEMENT AS TO FEDERALLY FUNDED RESEARCH

This invention was made with government support under National Science Foundation grant number EF-1105584 and National Institutes of Health Award Number F32GM103019. The government has certain rights in the invention.

### BACKGROUND OF THE INVENTION

The invention, in general, involves engineering photosynthesis in plants; in particular, C3 plants.

In photosynthetic organisms, D-ribulose-1,5-bisphosphate carboxylase/oxygenase (Rubisco) is the major enzyme assimilating atmospheric CO₂ into the biosphere (Andersson et al., Plant Physiol. Biochem. 46:275-291, 2008). Rubisco catalyses the incorporation of CO₂ into biological compounds in photosynthetic organisms (Andersson et al., Plant Physiol. Biochem. 46:275-291, 2008). Some variation in the catalytic properties of Rubisco from diverse sources is apparent. Harnessing this variation has the potential to confer superior photosynthetic characteristics to specific crops and environments (Zhu et al., Annu. Rev. Plant Biol. 61:235-261, 2010).

C4 plants, cyanobacteria, and hornworts have evolved forms of CO₂-concentrating mechanisms (CCM) that allow them to utilize forms of Rubisco that have higher catalytic rates and lower CO₂ affinity, whereas C3 plants, which lack a CCM, are constrained to express forms of Rubisco with higher CO₂ affinity but a relatively low rate of turnover (Whitney et al., Plant Physiol. 155:27-35, 2011). In plants, Rubisco is a L858 hexadecamer consisting of eight small subunits (SSU) and eight large subunits (LSU). Although the SSU genes are located in the nucleus, the LSU is encoded by the chloroplast genome, which has complicated previous attempts to engineer improvements in higher plant Rubisco (Whitney et al., Plant Physiol. 155:27-35, 2011; Dhingra et al., Proc. Natl Acad. Sci. USA 101:6315-6320, 2004).

### SUMMARY OF THE INVENTION

In general, the invention features a plant including a cyanobacterial ribulose-1,5,-bisphosphate carboxylase/oxygenase (Rubisco) which can assemble and fix carbon without an interacting protein (such as RbcX or CcmM35). In preferred embodiments, the plant is a C3 plant. Exemplary C3 plants include, without limitation, a variety of crop plants such as lettuce, tobacco, petunia, potato, tomato, soybean, carrot, cabbage, poplar, alfalfa, crucifers such as oilseed rape, and sugar beet. In yet other preferred embodiments, the cyanobacterial Rubisco assembles and fixes carbon in the cytoplasm of a plant cell. And in other preferred embodiments, the cyanobacterial Rubisco assembles and fixes carbon in a microcompartment in the cytoplasm. In other embodiments, the cyanobacterial Rubisco assembles and fixes carbon in a chloroplast of a plant cell. And in yet other preferred embodiments, the cyanobacterial Rubisco assembles and fixes carbon in a microcompartment in the chloroplast.

In another aspect, the invention features a method of expressing a cyanobacterial Rubisco in a plant cell, the method including expressing a cyanobacterial large Rubisco subunit (LSU) and a cyanobacterial small Rubisco subunit (SSU) in the plant cell which can assemble and fix carbon without an interacting protein (such as RbcX or CcmM35). In preferred embodiments, the plant is a C3 plant. In yet other preferred embodiments, the cyanobacterial Rubisco assembles and fixes carbon in the cytoplasm of the plant cell. And in other preferred embodiments, the cyanobacterial Rubisco assembles and fixes carbon in a microcompartment in the cytoplasm. In other embodiments, the cyanobacterial Rubisco assembles and fixes carbon in a chloroplast of the plant cell. And in yet other preferred embodiments, the cyanobacterial Rubisco assembles and fixes carbon in a microcompartment in the chloroplast.

And in another aspect, the invention features a method of engineering a plant expressing a cyanobacterial Rubisco, the method including (a) providing a plant cell that expresses a polypeptide having substantial identity to a cyanobacterial Rubisco LSU and a polypeptide having substantial identity to a cyanobacterial Rubisco SSU which can assemble and fix carbon without an interacting protein; and (b) regenerating a plant from the plant cell wherein the plant expresses the cyanobacterial Rubisco when compared to a corresponding untransformed plant. In preferred embodiments, the plant is a C3 plant. In yet other preferred embodiments, the cyanobacterial Rubisco assembles and fixes carbon in the cytoplasm of the plant cell. And in other preferred embodiments, the cyanobacterial Rubisco assembles and fixes carbon in a microcompartment in the cytoplasm. In other embodiments, the cyanobacterial Rubisco assembles and fixes carbon in a chloroplast of the plant cell. And in yet other preferred embodiments, the cyanobacterial Rubisco assembles and fixes carbon in a microcompartment in the chloroplast.

Cells and organisms described herein are, in general, “transformed” or “transgenic.” These terms accordingly refer to any cell (e.g., a host cell) or organism into which a recombinant or heterologous nucleic acid molecule (e.g., one or more DNA constructs) has been introduced. Thus, the nucleic acid molecule can be stably expressed (e.g., maintained in a functional form in the cell for longer than about three months) or non-stably maintained in a functional form in the cell for less than three months, or in other words is transiently expressed. Transgenic or transformed cells or organisms accordingly contain genetic material not found in untransformed cells or organisms. The term “untransformed” refers to cells that have not been through the transformation process.

The cells and organisms described herein are generally, but not limited to, plants (e.g., transgenic) or plant cells (e.g., transgenic), and the recombinant or heterologous nucleic acid molecules (e.g., a transgene) is inserted by artifice into the nuclear or plastidic genomes of the cells or organisms described herein. Progeny plant or plants deriving from (e.g., by propagating or breeding) the stable integration of heterologous genetic material into a specific location or locations within the nuclear genome or plastidic genome(s) or both of the original transformed cell are generally referred to as a “transgenic line” or a “transgenic plant line.” Transgenic plants or transgenic plant lines thus, for example, contain genetic material not found in an untransformed plant of the same species, variety, or cultivar.

The term “plant” as used herein includes whole plants or plant parts or plant components. By “plant part” or “plant component” is meant a part, segment, or organ obtained from, for example, an intact plant, plant tissue, or plant cell. Exemplary plant parts or plant components include, without limitation, somatic embryos, leaves, seeds, stems, roots, flowers, tendrils, fruits, scions, and rootstocks. Exemplary transformable plants include a variety of vascular plants (e.g., dicotyledonous and monocotyledonous plants as well as gymnosperms) and lower non-vascular plants. Preferably, the transgenic plant is a C3 plant, and in still other further preferred embodiments, chloroplasts of the C3 plant include heterologous genetic material such as a cyanobacterial Rubisco or a red-type Rubisco. In some embodiments, the cyanobacterial Rubisco or a red-type Rubisco or a Rhodobacter sphaeroides or a Halothiobacilus Rubisco or even a Limonium gibertii Rubisco or any combination thereof is housed in a recombinant microcompartment within the chloroplast of the plant or plant component. In some embodiments, the cyanobacterial Rubisco or a Rhodobacter sphaeroides or a Halothiobacilus Rubisco or a Limonium gibertii Rubisco or any combination thereof is housed in a microcompartment of the plant's cytoplasm.

By “plant cell” is meant any self-propagating cell bounded by a semi-permeable membrane and containing a plastid. Such a cell also requires a cell wall if further propagation is desired. Plant cell, as used herein includes, without limitation, suspension cultures of plant cells such as those obtained from embryos, meristematic regions, callus tissue, leaves, roots, shoots, gametophytes, sporophytes, pollen, and microspores.

As is disclosed herein, the cells and organisms include a cyanobacterial Rubisco (with or without an interacting protein such as RbcX or CcmM35) which assembles and fixes carbon. Cyanobacterial

Rubisco is preferably expressed in chloroplasts or, alternatively, in other preferred embodiments may be expressed in recombinant microcompartments in the chloroplasts. And in yet other preferred embodiments, a red-type Rubisco is expressed in chloroplasts or, alternatively, in other preferred embodiments may be expressed in recombinant microcompartments in the chloroplasts. Red-type Rubisco is found in photosynthetic bacteria, non-green algae, and phytoplankton. And in still other preferred embodiments, a Halothiobacilus Rubisco is expressed in chloroplasts or, alternatively, in other preferred embodiments may be expressed in recombinant microcompartments in the chloroplasts. In some embodiments, the aforementioned Rubiscos are expressed in microcompartments located in the plant's cytoplasm. Generation of such cells and organisms starts using standard transformation methodologies. The term “transformation” thus generally refers to the transfer of one or more recombinant or heterologous nucleic acid molecule (e.g., a transgene) into a host cell or organism. Methods for introducing nucleic acid molecules into host cells are well known in the art and include, for instance, those methods described herein. By “transgene” is meant any piece of a nucleic acid molecule (e.g., DNA or a recombinant polynucleotide) which is inserted by artifice into a cell, and becomes part of the genome of the organism which develops from that cell. Such a transgene may include a gene which is partly or entirely heterologous (i.e., foreign) to the transgenic organism, or may represent a gene having sequence identity to an endogenous gene of the organism. Exemplary useful genetic constructs such as SeLS, SeLSX, SeLSM35, and SeLSYM35 are described herein. Exemplary constructs for expressing red-type, Halothiobacillus rubiscos, Procholorococcus, or Limonium Rubiscos in plants are similar to those described for SeLS line. Here Rubisco large and small subunit genes in the SeLS construct are replaced with those from the corresponding Rubisco enzymes. Similar promoter, terminators, IEE, and other regulatory sequences are used in these constructs.

Plants expressing cyanobacterial Rubisco are preferably generated according to the methods described herein. In addition, plants expressing a red-type Rubisco or a Halothiobacilus Rubisco may be generated.

By cyanobacterial Rubisco is meant a Rubisco having substantial identity to a Rubisco found in a cyanobacterium such as Synechococcus or Procholorococcus (see, for example, FIGS. 9 and 10). Exemplary red-type and Halothiobacilus Rubiscos are described in FIG. 10. Other useful Rubiscos have substantial identity to a red-type and Halothiobacilus Rubisco described in FIG. 10.

Other useful Rubsicos include those from Limonium gibertii as disclosed in FIG. 10.

Microcompartments, besides improving photosynthesis, may also be used to reduce nitrogen demands on the plant that result from C3 plants having to invest a lot of nitrogen in Rubisco. Microcompartments can also encapsulate other oxygen-sensitive pathways in them, such as nitrogen-fixing enzymes. Microcompartments in general are useful for concentrating reactants and enzymes together to enhance production of a product. Recombinant microcompartments are typically generated utilizing recombinant polynucleotides which, in turn, are transcribed and translated resulting in the production of recombinant polypeptides. A “recombinant polynucleotide” is a polynucleotide that is not in its native state, e.g., the polynucleotide comprises a nucleotide sequence not found in nature, or the polynucleotide is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acid. A “recombinant polypeptide” is a polypeptide produced by translation of a recombinant polynucleotide. A “synthetic polypeptide” is a polypeptide created by consecutive polymerization of isolated amino acid residues using methods well known in the art. An “isolated polypeptide,” whether a naturally occurring or a recombinant polypeptide, is more enriched in (or out of) a cell than the polypeptide in its natural state in a wild-type cell, e.g., more than about 5% enriched, more than about 10% enriched, or more than about 20%, or more than about 50%, or more, enriched, i.e., alternatively denoted: 105%, 110%, 120%, 150% or more, enriched relative to wild type standardized at 100%. Alternatively, or additionally, the isolated polypeptide is separated from other cellular components with which it is typically associated, e.g., by any of the various protein purification methods known in the art.

By “polypeptide” or “protein” is meant any chain of amino acids, regardless of length or post-translational modification (for example, glycosylation or phosphorylation).

Described herein are various polynucleotides and polypeptides useful in producing not only cyanobacterial Rubsico (e.g., SeLS, SeLSX, SeLSM35, and SeLSYM35) in a plant but also microcompartments and carboxysomes including ccmP, CcmP, ccmO, CcmO, ccmK2, CcmK2, ccmL, CcmL, ccmM35, CcmM35, ccmM58, CcmM58, Synechococcus LSU (Rubisco large subunit) nucleotide sequence, Synechococcus LSU (Rubisco large subunit), Synechococcus SSU (Rubisco small subunit) nucleotide sequence, Synechococcus SSU (Rubisco small subunit), rbcX, RbcX, ccmM35, CcmM35, ccmK3, CcmK3, ccmK4, CcmK4, ccaA, CcaA (carbonic anhydrase), ccmN, and CcmN (FIG. 9). Rubiscos substantially identical to those described in FIG. 10 may also be produced in plants, with or without microcompartments, as described herein.

It is understood that polynucleotides and polypeptides having substantial identity to such molecules are also useful in the methods disclosed herein. By “having substantial identity to” or by “substantially identical to” is meant a polynucleotide or polypeptide exhibiting at least 50% or 60%, preferably 70%, 75%, 85%, or 85%, more preferably 90%, and most preferably 95%, 96%, 97%, 98%, and 99% homology (or identity) to a reference nucleic acid or amino acid sequence. For nucleic acids, the length of comparison sequences will generally be at least 50 nucleotides, preferably at least 60 nucleotides, more preferably at least 75 nucleotides, and most preferably 110 nucleotides. For polypeptides, the length of comparison sequences will generally be at least 16 amino acids, preferably at least 20 amino acids, more preferably at least 25 amino acids, and most preferably 35 amino acids. Sequence identity is typically measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various substitutions, deletions, substitutions, and other modifications. Conservative substitutions typically include substitutions within the following groups: glycine alanine; valine, isoleucine, leucine; aspartic acid, glutamic acid, asparagine, glutamine; serine, threonine; lysine, arginine; and phenylalanine, tyrosine.

Other features and advantages of the invention will be apparent from the following description of the preferred embodiments thereof, and from the claims.

### BRIEF DESCRIPTION OF THE DRAWINGS

FIGS. 1a-1c show the replacement of the tobacco chloroplast rbcL with cyanobacterial genes. Panel a shows gene arrangements of the rbcL locus in the wild-type, SeLSX, SeLSM35, and SeLS tobacco lines. Endogenous chloroplast DNA elements are shown in grey and the newly introduced segments in black. The intergenic regions IG1, IG2, IG3 and IG4 include TpetD(At)-IEE-SD, TpsbA(At)-IEE-SD, Trps16(At)-IEE-SD and TpsbA(At)-IEE-SD18 respectively, where TpetD, TpsbA and Trps16 are the terminator sequences following the corresponding genes and At stands for the chloroplast of Arabidopsis thaliana as the source of these sequences. The selectable marker operon (SMO) includes LoxP-PpsbA-aadA-Trps16-LoxP, where PpsbA stands for the promoter of the psbA gene. The probe recognizes the rbcL promoter (PrbcL) region. The Nhel and Ndel sites used in the DNA blot along with the lengths of the expected DNA fragments detected by the probe are indicated. DIG, digoxygenin. Panel b shows DNA blot analysis of wild-type, SeLSX and SeLSM35 lines digested with Ndel and Nhel. Panel c shows analyses of RT-PCR products of 6 genes. Nt-rbcL is the only tobacco (Nt, Nicotiana tabacum) gene; all other genes are the transgenes introduced into the tobacco chloroplast genome. X=rbcX, M=ccmM35.

FIGS. 2a-2d show the cyanobacterial proteins in tobacco chloroplasts. Panel a shows Coomassie-stained gel and immunoblot of 14 pg of total leaf protein from wild-type (WT), SeLSX and SeLSM35 tobacco lines. Immunoblots were probed with the antibodies indicated. Molecular mass (kDa) of standard proteins are shown. Asterisk symbol indicates molecular mass of tobacco SSU; dagger symbol indicates molecular mass of cyanobacterial SSU. c, cyanobacteria; t, tobacco. Panels b-d are electron micrographs of leaf sections showing the localization of Rubisco in the stroma of mesophyll chloroplasts of wild-type (b), SeLSX (c) and SeLSM35 (d) tobacco lines. Leaf tissues were prepared by high pressure freeze fixation (HPF) in combination with immunogold labelling using an antitobacco Rubisco antibody (b) or an anti-cyanobacterial Rubisco antibody (c, d) and a secondary antibody conjugated with 10 nm gold particles, which are indicated with either black circles or arrows. Scale bars, 500 nm (top panels in b, d) and 200 nm (c and the bottom panels in b, d).

FIGS. 3a-3c show the Rubisco and CcmM35 content of SeLSM35 tobacco leaves. The stated concentrations of purified Se Rubisco (a) and CcmM35 (b) proteins were used as standards. a, Immunoblot using an antibody against cyanobacterial LSU (top) and the standard curve used to estimate the amount of cyanobacterial Rubisco in samples S1-S3 extracted from SeLSM35 tobacco leaves (bottom). b, Immunoblot using an antibody against CcmM (top) and the standard curve used to estimate the amount of CcmM35 in samples S4-S6 extracted from SeLSM35 tobacco leaves (bottom). The band intensities in the two standard curves were obtained with ImageJ software and the standard curves with Microsoft Excel. c, The absolute and relative amounts (mean ±standard deviation) of CcmM35 and cyanobacterial Rubisco in SeLSM35 tobacco line from two separate measurements. Each Rubisco holoenzyme is assumed to be composed of 8 LSU and an unknown quantity of SSU.

FIG. 4 shows the electron micrographs of ultrathin sections of leaf mesophyll cells from the chloroplast transformant SeLSM35. Large compartments containing cyanobacterial Rubisco and CcmM35 in the chloroplast stroma are indicated by black arrows. Leaf tissues were prepared by high pressure freeze fixation (HPF) in combination with immunogold labeling using an antibody against CcmM. A secondary antibody conjugated with 10-nm gold particles was used for the labelling. Scale bars, 500 nm. FIGS. 5a-5e show the phenotype of the wild-type and transplastomic tobacco lines. Plants were grown at atmospheric CO₂ level about 9,000 p.p.m. Panels a-e are pictures showing 6-week-old wild-type (a), SeLSX (b), and SeLSM35 (c); and 10-week old SeLSX (d) and SeLSM35 (e) tobacco lines grown in the same conditions. Scale bars, 5 cm.

FIG. 6 shows the carboxylase activities at different ¹⁴CO₂ concentrations. CO₂ fixation by crude leaf homogenates from tobacco lines expressing cyanobacterial Rubisco (SeLSX and SeLSM35) and wild-type tobacco (WT). The rates of carboxylase activity (mol CO₂ fixed per mol active sites per s) at each point of the curves are the mean ±standard deviation of the 2, 4 and 6 min data obtained in two independent assays at different CO₂ concentrations (125 μM, 250 μM, 640 μM).

FIGS. 7a-7b show Rubisco-specific ¹⁴CO₂ fixation by crude leaf homogenates from tobacco lines expressing cyanobacterial Rubisco (SeLSX and SeLSM35) and wild-type tobacco (WT). a, Carboxylase activity assayed with (+) and without (−) RuBP. b, Carboxylase activity assayed with (+) and without (−) the inhibitor CABP. The rates of carboxylase activity (mols fixed per mol act sites per s) are the mean ±standard deviation derived from the 2, 4 and 10 min data obtained in assays at 125 μM CO₂ (corresponding to 10 mM NaH¹⁴CO₃, at pH 8.0).

FIG. 8 shows a TEM image of SeLS line showing a chloroplast and the cyanobacterial Rubisco detected by immunogold labeling.

FIG. 9 shows the nucleotide and amino acid sequences for ccmP, CcmP, ccmO, CcmO, ccmK2, CcmK2, ccmL, CcmL, ccmM35, CcmM35, ccmM58, CcmM58, Synechococcus LS (Rubisco large subunit) nucleotide sequence, Synechococcus LS (Rubisco large subunit), Synechococcus SS (Rubisco small subunit) nucleotide sequence, Synechococcus SS (Rubisco small subunit), rbcX, RbcX, ccmM35, CcmM35, ccmK3, CcmK3, ccmK4, CcmK4, ccaA, CcaA (carbonic anhydrase), ccmN, and CcmN.

FIG. 10 shows sequences of Synechococcus elongatus (strain PCC 7942), Prochlorococcus marinus, Halothiobacillus neapolitanus c2, Rhodobacter sphaeroides, and Limonium gibertii Rubiscos.

FIG. 11 shows that the rbc/gene in tobacco chloroplasts was replaced with synthetic operons containing cyanobacterial genes. Different combinations of cyanobacterial genes were inserted to replace the tobacco rbcL gene in SeLS, SeLSX, SeLSM35 and SeLSYM35 tobacco lines. Three terminators from the chloroplast genome of Arabidopsis thaliana (At-Trps16, At-TpetD and At-TpsbA), intercistronic expression elements (IEE) and Shine Dalgarno sequences (SD or SD18) are inserted between the cyanobacterial genes.

FIGS. 12a-12c show the Replacement of the Nt-rbcl gene with synthetic operons containing cyanobacterial genes. (a) DNA blot analysis of 1 μg total DNA from each tobacco line digested with Ndel and Nhel restriction enzymes indicates the absence of wild-type DNA fragment in the four transplastomic tobacco lines. (b) RNA blot analysis of the wild-type and four transplastomic tobacco lines confirms the absence of transcripts containing Nt-rbcL gene in the four transplastomic tobacco lines. (c) RNA blot analysis with the RNA probe to detect transcripts containing the aadA selectable marker gene shows the transcripts from PpsbA promoter located immediately upstream as well as the read-through transcripts from the PrbcL promoter. Please refer to FIG. 11 for the configurations of operons in different transformants and FIG. 13 for the identification of transcripts resulting from PrbcL promoters.

FIGS. 13a-13d show RNA blot analyses show different patterns of transcript processing and concentrations in the four chloroplast transformants. Most transcripts expected to arise from the rbcL locus of the four chloroplast transformant lines due to incomplete processing at the IEE sites were detected on the RNA blots. Each blot was run with triplicate samples obtained from three different plants grown under the same conditions. The transcripts identified are indicated on the left side of the bands in each blot. Each transcript is named with the abbreviations of the transgenes included in that particular transcript (L=Se-rbcL, S=Se-rbcS, A=aadA, X=Se-rbcX, M35=Se-ccmM35 and YM35=YFP:Se-ccmM35). The scale bar in (a) also applies to (b), (c) and (d).

FIGS. 14a-14c shows the expression of Se7942 Rubisco in the four chloroplast transformants. (a) SDS-polyacrylamide gel stained with Coomassie (far left) together with immunoblots probed with an anti-tobacco LSU, anti-tobacco SSU, anti-Se LSU and anti-CcmM antisera indicate expression of cyanobacterial proteins and absence of tobacco Rubisco subunits. In all cases, 15 μg of total leaf protein from the indicated sources were loaded. (b) Tobacco SSU was detected in the immunoblot of Rubisco samples extracted from wild-type and the four tobacco chloroplast transformant lines. Rubisco complexes were extracted with Triton X-100 and concentrated using HiTrap-Q anion-exchange columns. (c) Native polyacrylamide gel stained with Coomassie Blue (far left) together with immunoblots probed with antibodies show the assembly of hexadecameric Rubisco complexes in the four tobacco chloroplast transformant lines. In all cases, 20 μg of total leaf protein from the indicated sources were loaded. Indicated bands correspond to tobacco Rubisco holoenzyme (Hₜ); Se7942 Rubisco holoenzyme (H_{c}); YFP-CcmM35 (YM35); CcmM35 (M35) and an unknown cross-reacting protein from tobacco (*). The mass of protein standards (M) are indicated (thyroglobulin (669 kDa); wheat Rubisco (550 kDa); BSA dimer (132 kDa); BSA (66 kDa); CcmM35His (37.5 kDa)).

FIG. 15 shows the localization of Rubisco in the chloroplast stroma of the wild-type and the four transplastomic tobacco lines. Electron micrographs of ultrathin sections of leaf mesophyll cells prepared by high pressure freeze fixation and freeze substitution. Ultrathin sections were probed with the indicated primary antibody and a secondary antibody conjugated with 10 nm gold particles (black circles). The labelling revealed the diffuse localization of cyanobacterial Rubisco into the chloroplast stroma of SeLS and SeLSX, whereas in SeLSYM35 and SeLSM35 the cyanobacterial enzyme localize in large aggregates with ccmM35. Scale bars=500 nm.

FIG. 16 shows the YFP-CcmM35 bodies in chloroplasts within leaf tissue of the SeLSYM35 tobacco line. The excitation wavelengths for YFP and chlorophyll a were 514 nm and 488 nm, respectively. Emitted spectra of 520-560 nm and 650-720 nm were collected for YFP (shown in green) and chlorophyll a (shown in red), respectively.

FIGS. 17a-17b show the determination and correspondence of Rubisco kinetic parameters with leaf photosynthesis. (a) Kinetic parameters of Se7942 Rubisco extracted from transgenic tobacco lines grown in air supplemented with 0.9% (v/v) CO₂ and of Rubisco from wild-type tobacco grown at ambient CO₂. Vₘₐₓ^{C} and Vₘₐₓ^{O}: rate constants for carboxylase and oxygenase activities respectively; K_{M}^{C} and K_{M}^{O}: Michaelis constants for CO₂ and O₂ respectively; S_{c/c}: specificity factor. Values are the mean ±sd of three independent determinations, and the S_{c/c} is the mean ±sd of five replicate determinations. (b) Rates of CO₂ assimilation using attached leaves of WT and transgenic tobacco. Results are expressed relative to leaf area (left); and Rubisco active site concentration (right). The illustrated (solid) lines were generated using a biochemical model (Farquhar et al., 1980) incorporating the kinetic parameters of (a) determined for WT (blue), SeLS (purple) and SeLSYM35 (red) tobacco lines. The modeled curves were also optimized to minimize deviation from the observed points by varying the leaf Rubisco content (broken lines). Data points are the mean values from three plants per line. Error bars indicate standard error, (omitted for clarity from the transformed lines, but shown in FIG. 18).

FIG. 18 shows the A-Ci curves from attached leaves containing the wild type and cyanobacterial Rubisco expressed in tobacco chloroplasts. Carrier gas composition (v/v): 98% N₂, 2% O₂ (open symbols); 79% N₂, 21% O₂ (filled symbols). Points show the mean and standard error of 3 plants per line.

FIGS. 19a-19c show the SeLS and SeLSYM35 grow significantly faster than SeLSX and SeLSM35 tobacco lines. (a) Wild-type tobacco was grown at normal (400 ppm) and 3% (v/v) CO₂ (denoted by “*”) while the SeLS, SeLSX, SeLSM35 and SeLSYM35 lines were all grown in air containing 3% (v/v) CO₂. Pictures show the plants at the indicated times (days) after germination. (b) Each plant was pictured at the indicated times, when the total leaf area reached ˜5,000 cm². Scale bars (white): 15 cm throughout. (c) From left to right: the increase in leaf area with time; the increase in leaf area with leaf number; and the increase in leaf area with plant height. Three weeks after germination, the total leaf area, number of leaves and plant height were recorded every 2-3 days until a total plant leaf area of ˜5,000 cm² was attained. The data are expressed as mean values ±sd of three plants per line.

FIGS. 20a-20f show the quantifications, fresh weight and dry weight of chloroplast transformants and wild type controls. (a) The four chloroplast transformants have lower total soluble protein compared to the wild-type plants. (b) All plants have similar amounts of total proteins. (c) The levels of chlorophylls a and b were higher in SeLS and SeLSYM35 plants compared to SeLSX and SeLSM35 plants. (d) The Rubisco contents in SeLS and SeLSX are dramatically lower than the wild-type, SeLSM35 and SeLSYM35 plants. (e) SeLSM35 and SeLSYM35 have greater leaf fresh weight than the wild-type, SeLS and SeLSX plants. (f) The SeLSYM35 and wild-type plants grown in 3% (v/v) CO₂ have greater leaf dry weight. WT plants were grown at 400 ppm CO₂ whereas WT* and the four chloroplast transformants were grown at 3% (v/v) CO₂. The data are means ±sd of 9 leaves from different positions in the plant canopy ranging from the youngest fully expanded to the oldest non-senescent leaf, collected from each of three plants per genotype.

FIGS. 21a-21c show the quantification of total leaf proteins and chlorophylls. Wild type tobacco (WT) grown at atmospheric CO₂ (400ppm) together with wild type tobacco (WT*) and four transgenic lines (SeLS, SeLSX, SeLSM35 and SeLSYM35) grown in air containing 3% (v/v) CO₂ are indicated. (a) Total soluble protein; (b) Total leaf protein; (c) Chlorophyll a and b content. The values are means ±sd of three leaves from each of the indicated positions (Top, upper; Mid, middle; Bot, bottom). Each leaf was collected from a separate plant (n=3).

FIGS. 22a-22c show the quantification of Rubisco and leaf fresh and dry weights. Wild type tobacco (WT) grown at atmospheric CO₂ (400ppm) together with wild type tobacco (WT*) and four transgenic lines

(SeLS, SeLSX, SeLSM35 and SeLSYM35) grown in air containing 3% (v/v) CO₂ are indicated. (a) Rubisco content; (b) leaf fresh weight; (c) leaf dry weight. The values are means ±sd of three leaves from the indicated positions on each plant (Top, upper; Mid, middle; Bot, bottom of the canopy). Each leaf was collected from a separate plant (n=3).

FIG. 23 shows the sequence of chloroplast transformation construct SeLSX.

FIG. 24 shows the sequence of chloroplast transformation construct SeLSM35.

FIG. 25 shows the sequence of chloroplast transformation construct SeLS.

FIG. 26 shows the sequence of chloroplast transformation construct SeLSYM35.

### DETAILED DESCRIPTION

Below we describe engineering plants to express a functional cyanobacterial form of Rubisco. Here we replaced chloroplast DNA that encodes the large subunit of Rubisco in the plant with that encoding the cyanobacterial enzyme. In particular, we co-expressed the cyanobacterial Rubisco with proteins that are involved in the Rubisco's assembly. We found that co-expression of cyanobacterial Rubisco with either the RbcX protein or the carboxysomal protein, CcmM35, were equally effective at forming functional Rubisco. Additionally, we found that that co-expression of cyanobacterial Rubisco alone resulted in assembly of an enzyme that is capable of fixing carbon.

Plant Expression Constructs

The construction of nuclear expression cassettes for use in virtually any plant, such as in C3 plants, is well established.

Expression cassettes are DNA constructs where various promoter, coding, and polyadenylation sequences are operably linked. In general, expression cassettes typically include a promoter that is operably linked to a sequence of interest which is operably linked to a polyadenylation or terminator region. In certain instances including, but not limited to, the expression of transgenes in a plant, it may also be useful to include an intron sequence to enhance expression. A variety of promoters can be used as well. One broad class of useful promoters is referred to as “constitutive” promoters in that they are active in most plant organs throughout plant development. For example, the promoter can be a viral promoter such as a CaMV35S promoter. The CaMV35S promoters are active in a variety of transformed plant tissues and most plant organs (e.g., callus, leaf, seed and root). Enhanced or duplicate versions of the CaMV35S promoters are particularly useful as well. Other useful promoters are known in the art.

Promoters that are active in certain plant tissues (i.e., tissue specific promoters) can also be used to drive expression of a carboxysome protein disclosed herein to facilitate production of a microcompartment in a plant cell. Transcriptional enhancer elements can also be used in conjunction with any promoter that is active in a plant cell or with any basal promoter element that requires an enhancer for activity in a plant cell. Transcriptional enhancer elements can activate transcription in various plant cells and are usually 100-200 base pairs long. The enhancer elements can be obtained by chemical synthesis or by isolation from regulatory elements that include such elements, and can include additional flanking nucleotides that contain useful restriction enzyme sites to facilitate subsequence manipulation. Enhancer elements can be typically placed within the region 5′ to the mRNA cap site associated with a promoter, but can also be located in regions that are 3′ to the cap site (i.e., within a 5′ untranslated region, an intron, or 3′ to a polyadenylation site) to provide for increased levels of expression of operably linked genes. Such enhancers are well known in the art. A polyadenylation signal provides for the addition of a polyadenylate sequence to the 3′ end of the RNA. The Agrobacterium tumor-inducing (Ti) plasmid nopaline synthase (NOS) gene and the pea ssRUBISCO E9 gene 3′ untranslated regions contain polyadenylate signals and represent non-limiting examples of such 3′ untranslated regions that can be used in constructing an expression cassette. It is understood that this group of exemplary polyadenylation regions is non-limiting and that one skilled in the art could employ other polyadenylation regions that are not explicitly cited here.

Additionally 5′ untranslated leader sequences can be operably linked to a coding sequence of interest in a plant expression cassette. Thus the plant expression cassette can contain one or more 5′ non-translated leader sequences which serve to increase expression of operably linked nucleic acid coding sequences encoding any of the polypeptides described herein.

Sequences encoding peptides that provide for the localization of any of the polypeptides described herein in to plastids can be operably linked to the sequences that encode the particular polypeptide. Transit sequences for incorporating nuclear-encoded proteins into plastids are well known in the art.

It is anticipated that any of the aforementioned plant expression elements can be used with a polynucleotide designed so that they will express one or more of the polypeptides encoded by any of the polynucleotides described herein in a plant or a plant part. Plant expression cassettes including one or more of the polynucleotides described herein which encode one or more of their respective polypeptides that will provide for expression of one or more polypeptides in a plant are provided herein.

The DNA constructs that include the plant expression cassettes described above are typically maintained in various vectors. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically include sequences that permit replication in both E. coli and Agrobacterium as well as one or more “border” sequences positioned so as to permit integration of the expression cassette into the plant chromosome. Selectable markers encoding genes that confer resistance to antibiotics are also typically included in the vectors to provide for their maintenance in bacterial hosts.

Much of the discussion above, which concerns nuclear transformation, is relevant to introduction of genes into plastids and chloroplasts, but there are some differences (see, for example, Hanson et al., Journal of Experimental Botany 64: 731-742, 2013). For example, polyadenylation signals are not placed on chloroplast transgenes; instead plastid 3′ stability sequences must be incorporated. Unlike typical Agrobacterium-mediated nuclear transformation, there is a simple method to ensure proper targeting of a transgene to a location of interest within the plastid genome, by surrounding the transgene with plastid DNA sequences so that homologous recombination will occur. Selection of proper promoter and 5′ untranslated region sequences is according to methods known in the art, and sometimes a suitable “downstream box” at the beginning of the translated region is needed to modulate expression (see, for example, Gray et al., Biotechnology and Bioengineering 102: 1045-1054, 2009). Furthermore, because plastid genes can be transcribed in operons, to optimize expression an intercistronic expression element (IEE) can be used so that monocistronic transcripts are obtained for better expression levels (see, for example, Zhou et al., The Plant Journal: for Cell and Molecular Biology 52: 961-972, 2007). No plastid transit sequence is needed on the plastid transgene since expression occurs from within the plastid.

In other embodiments, the amount of protein that accumulates due to expression from a chloroplast transgene can also be influenced by the identity of the second amino acid encoded by the transgene, due to its effect on protein stability (see, for example, Apel et al., Plant Journal 63: 636-650, 2010).

Plants and Methods for Obtaining Plants including Carboxysome Proteins

Methods of obtaining a plant (or a plant part) including a recombinant microcompartment are also optionally provided by this invention. First, expression vectors suitable for expression of any of the polypeptides disclosed herein are introduced into a plant, a plant cell or a plant tissue using transformation techniques according to standard methods well known in the art. Next a plant containing the plant expression vector is obtained by regenerating that plant from the plant, plant cell or plant tissue that received the expression vector. The final step, if desired, is to obtain a plant that expresses a carboxysome protein and, preferably, a microcompartment.

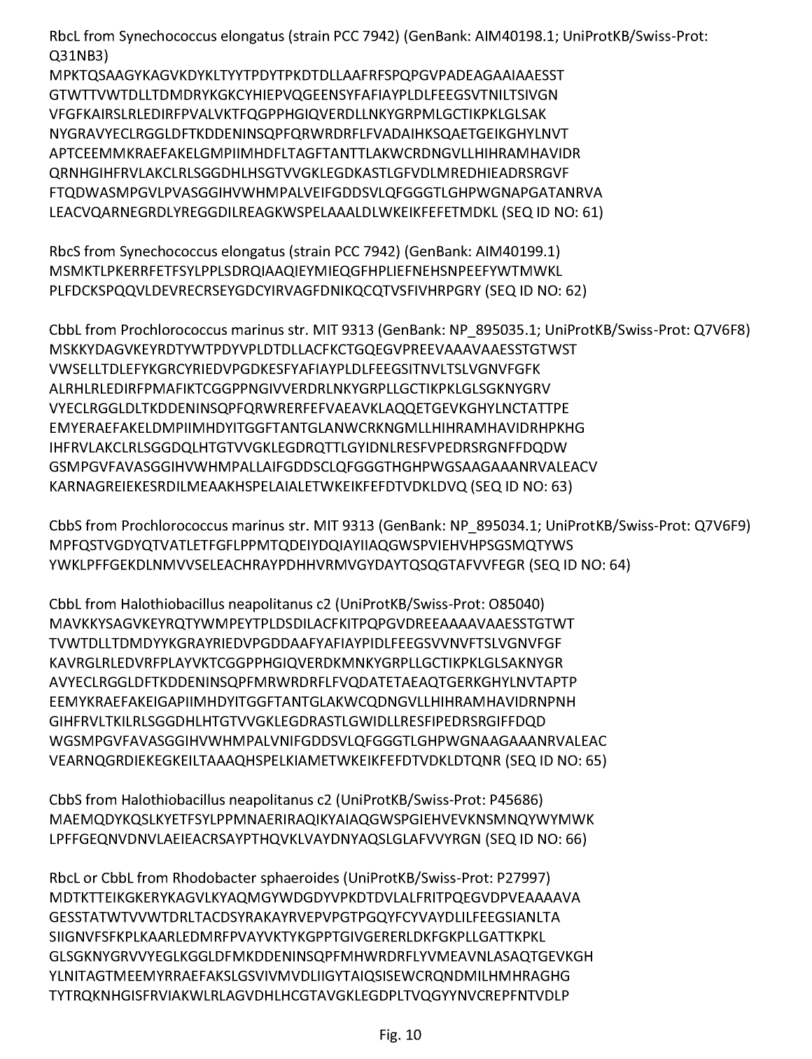

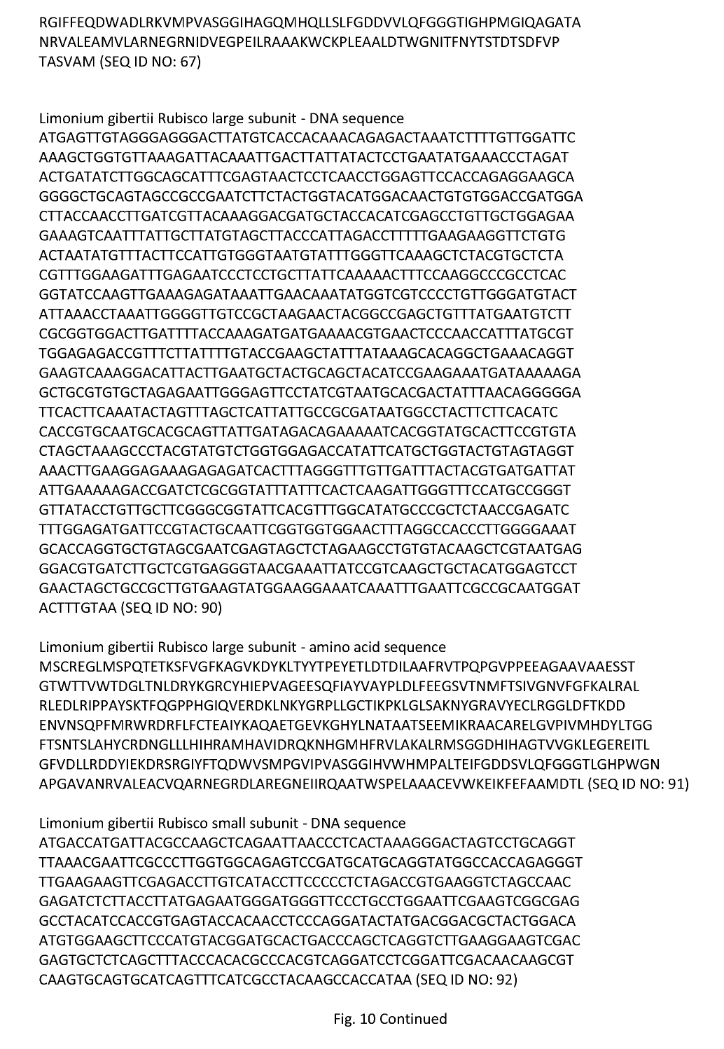

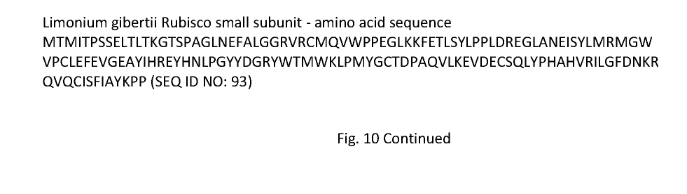

In particular, a microcompartment that includes a protein having substantial identity to CcmK2, a protein having substantial identity to CcmL, a protein having substantial identity to CcmO, a protein having substantial identity to CcmN, a protein having substantial identity to CcmM58, and a protein having substantial identity to CcaA is useful for housing a cyanobacterial Rubisco that includes a protein having substantial identity to cyanobacterial Rubisco LSU, and a protein having substantial identity to cyanobacterial Rubisco SSU, as well as Rubiscos having substantial identity to any one shown in FIG. 10. The amount of protein that accumulates due to expression from a chloroplast transgene can also be influenced by the identity of the second amino acid encoded by the transgene, due to its effect on protein stability (see, for example, Apel et al., Plant Journal 63: 636-650, 2010).

Plant expression vectors can be introduced into the chromosomes of a host plant via methods such as Agrobacterium-mediated transformation, particle-mediated transformation, DNA transfection, or DNA electroporation, or by so-called whiskers-mediated transformation. Exemplary methods of introducing transgenes are well known to those skilled in the art including those described herein.

Those skilled in the art will further appreciate that any of these gene transfer techniques can be used to introduce the expression vector into the chromosome of a plant cell, a plant tissue, a plant, or a plant part. When the plant expression vector is introduced into a plant cell or plant tissue, the transformed cells or tissues are typically regenerated into whole plants by culturing these cells or tissues under conditions that promote the formation of a whole plant (i.e., the process of regenerating leaves, stems, roots, and, in certain plants, reproductive tissues). The development or regeneration of transgenic plants from either single plant protoplasts or various explants is well known in the art. This regeneration and growth process typically includes the steps of selection of transformed cells and culturing selected cells under conditions that will yield rooted plantlets. The resulting transgenic rooted shoots are then planted in an appropriate plant growth medium such as soil. Transgenic plants having incorporated into their genome transgenic DNA segments encoding one or more of the polypeptides described herein are within the scope of the invention. It is further recognized that transgenic plants containing the DNA constructs described herein, and materials derived therefrom, may be identified through use of PCR or other methods that can specifically detect the sequences in the DNA constructs.

Once a plant is regenerated or recovered, a variety of methods can be used to identify or obtain a transgenic plant that includes one or more of the polypeptides described herein as well as includes a carboxysome. One general set of methods is to perform assays that measure the amount of the polypeptide that is produced. Alternatively, the amount of mRNA produced by the transgenic plant can be determined to identify plants that express of the polypeptide. Standard microscopic methods are also useful to identify plants engineered to include carboxysomes.

### EXAMPLE 1

In the following example, we describe two transplastomic tobacco lines with functional Rubisco from the cyanobacterium Synechococcus elongatus PCC7942 (Se7942). We knocked out the native tobacco gene encoding the large subunit of Rubisco by inserting the large and small subunit genes of the Se7942 enzyme, in combination with either the corresponding Se7942 assembly chaperone, RbcX, or an internal carboxysomal protein, CcmM35, which incorporates three small subunit-like domains (Saschenbrecker et al., Cell 129: 1189-1200, 2007; Long et al., J. Biol. Chem. 282:29323-29335, 2007). Se7942 Rubisco and CcmM35 formed macromolecular complexes within the chloroplast stroma, mirroring an early step in the biogenesis of cyanobacterial β-carboxysomes (Cameron et al., Cell 155: 1131-1140, 2013; Chen et al., PLoS ONE 8:e76127, 2013). Additionally, we describe a third transplastomic tobacco line with functional Rubisco from Se7942, without RbcX or the internal carboxysomal protein, CcmM35. All three transformed lines were photosynthetically competent, supporting autotrophic growth, and their respective forms of Rubisco had higher rates of CO₂ fixation per unit of enzyme than the tobacco control.

SeLSX, SeLSM35, and SeLS

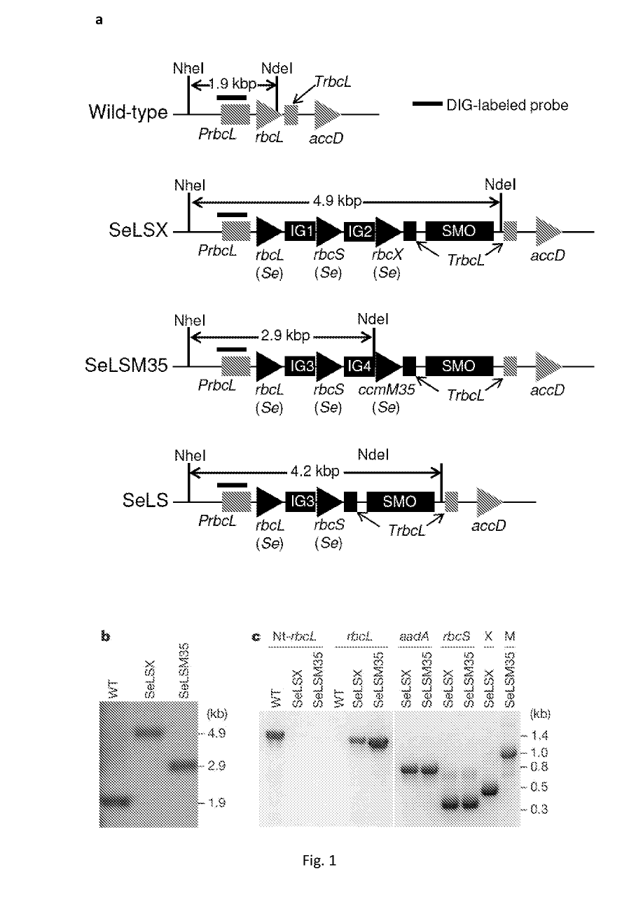

To test whether cyanobacterial LSU and SSU can assemble into a functional enzyme within higher plant chloroplasts, we generated two transplastomic tobacco lines, named here SeLSX and SeLSM35, using the biolistic delivery system (Maliga et al., Method Mol. Biol. 1132:147-163, 2014), to express the two Rubisco subunits from Se7942 along with either RbcX or CcmM35, respectively. A construct, SeLS, was also engineered to assemble Rubisco without RbcX or CcmM35. In each chloroplast transformant, three genes or two were co-transcribed from the tobacco rbcL promoter. Each downstream gene was preceded by an intercistronic expression element (IEE) and a Shine-Dalgarno sequence (SD) and equipped with a terminator to facilitate processing into translatable monocistronic transcripts (Zhou et al., Plant J. 52: 961-972, 2007; Dreschel et al., Nucleic Acids Res. 39:1427-1438, 2011)(FIG. 1a).

The three vectors we constructed were designed to replace the tobacco rbcL gene with the foreign DNA.

To determine whether all chloroplasts in each plant contained the transgenic locus rather than endogenous tobacco rbcL, we examined blots of total leaf DNA digested with restriction enzymes that would produce restriction fragment-length polymorphisms between the wild-type and transgenic loci (FIG. 1 b). We found that shoots arising after two rounds on selective medium were homoplasmic for the transgene locus, lacking the fragment corresponding to the wild-type chloroplast genome (FIG. 1 b). To verify these observations, we performed reverse transcription and PCR (RT-PCR) and observed no cDNA derived from the native rbcL transcript, whereas cDNAs produced from aad A, the selectable marker gene, and the cyanobacterial genes were detected (FIG. 1c).

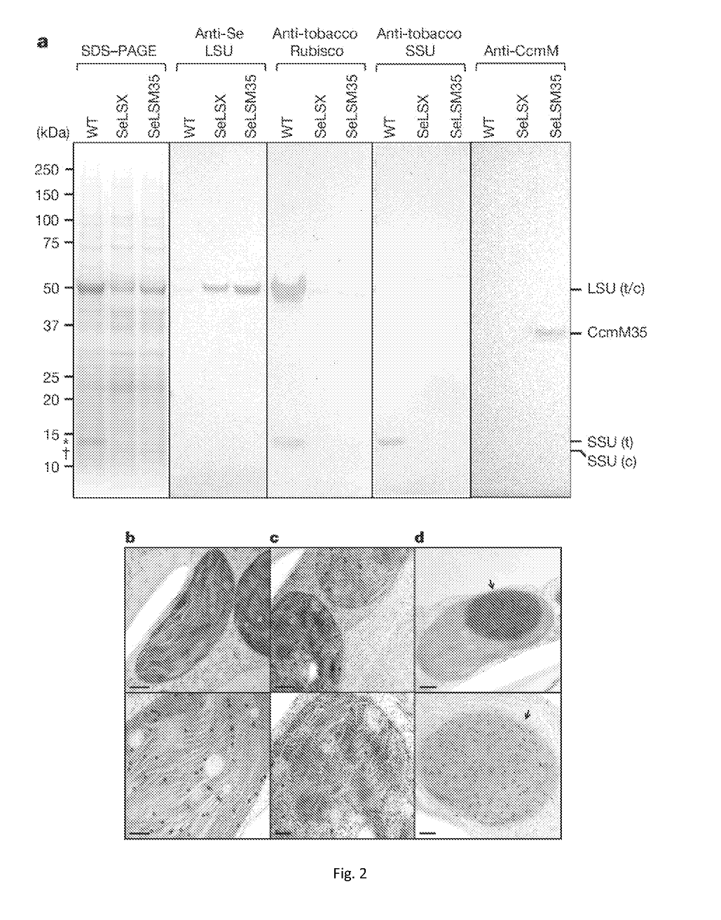

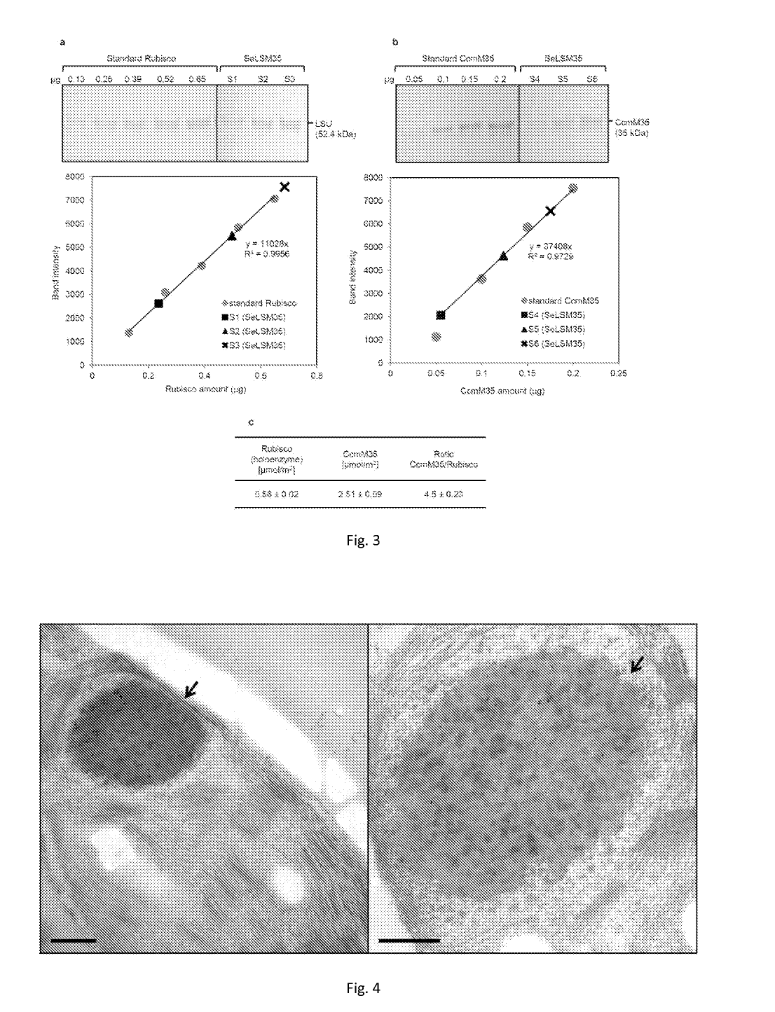

To observe the expression of the cyanobacterial proteins, we extracted total leaf proteins and examined them by SDS-PAGE and immunoblots. In Coomassie-stained gels, we detected protein bands at the predicted molecular masses of ˜52 kDa for the LSU and ˜13 kDa for the SSU of the cyanobacterial Rubisco in SeLSX and SeLSM35 samples, whereas wild-type tobacco exhibited a protein of the expected and distinct SSU mass of ˜15 kDa (FIG. 2a). Immunoblots probed with antibodies specific for either the cyanobacterial LSU, tobacco Rubisco, tobacco SSU or cyanobacterial CcmM35 verified the presence of cyanobacterial proteins in the two transformants and tobacco Rubisco only in the wild-type plant (FIG. 2a). Although no engineering of tobacco SSU genes was performed in the transgenic lines, tobacco SSU protein was undetectable, as expected, as its stability is known to be severely affected in the absence of a compatible LSU (Kanevski et al., Plant Physiol. 119: 133-142, 1999; Whitney et al., Proc. Natl. Acad. Sci. USA 98: 14738-14743, 2001). The absence of the tobacco SSU in the transformants also indicated that it could not form a stable complex with the cyanobacterial LSU. The estimated stoichiometry of CcmM35 per Rubisco holoenzyme in SeLSM35 transformant is about 4.5, which is consistent with the values reported for cyanobacteria (FIG. 3) (Long et al., Photosynth. Res. 109: 33-45, 2011).

In order to observe the configuration of the cyanobacterial Rubisco in the two transgenic lines, we examined the plant material by transmission electron microscopy (TEM) in combination with immunogold labelling.

Although the enzyme was localized to the chloroplast stroma in both transgenic lines, we observed markedly different patterns of molecular organization. In leaves of the SeLSX line, the cyanobacterial

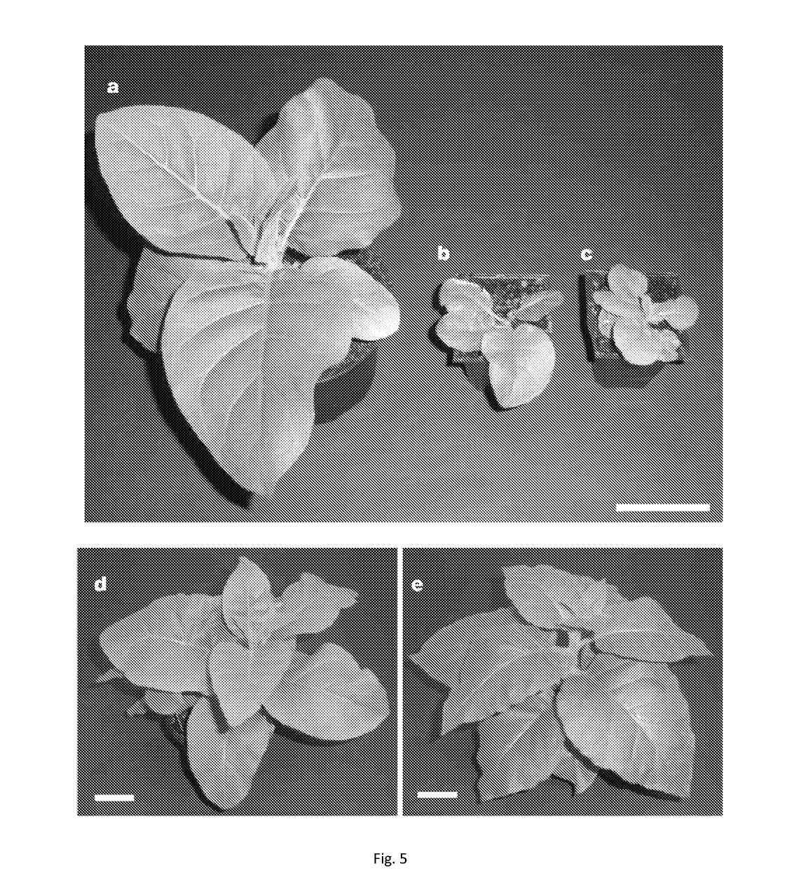

Rubisco showed a diffuse localization similar to endogenous Rubisco in wild-type tobacco (FIG. 2b, c). In contrast, in the SeLSM35 line, in which the Rubisco is co-expressed with CcmM35, the proteins were aggregated into a giant complex in each chloroplast (FIG. 2d and FIG. 4).

In Se7942, CcmM35 is translated from an internal ribosome entry site of the ccmM transcript, which also produces the full-length protein, CcmM58, with an additional amino-terminal domain (Long et al., Plant Physiol. 153: 285-293, 2010). Previous estimation of protein ratios suggested that Rubisco in Se PCC7942 probably exists as L8S5 units crosslinked by the SSU-like domains of CcmM35 resulting in their paracrystalline arrangement in the lumen of β-carboxysomes (Long et al., Photosynth. Res. 109: 33-45, 2011). The cyanobacterial mutant lacking CcmM58 produces large electrondense bodies of 300-500 nm with a rectangular cross-section composed of Rubisco and CcmM35 (Long et al., Plant Physiol. 153: 285-293, 2010). However, the structures formed inside chloroplasts are generally rounded in appearance without apparent internal order. This discrepancy probably arises from different ratios of Rubisco and CcmM35 or additional carboxysomal components potentially present in the cyanobacterial bodies. Remarkably, the structures observed in chloroplasts are highly similar in appearance to procarboxysomes recently identified as an important early stage in the carboxysome assembly (Cameron et al., Cell 155: 1131-1140, 2013) and likely facilitate future attempts to assemble β-carboxysomes in chloroplasts through expression of other essential components.

The specificity of the carboxylase activity of cyanobacterial Rubisco relative to its competing oxygenase activity (specificity factor) is known to be lower than that in higher plants, making it more sensitive to the inhibitory effects of oxygen than tobacco Rubisco (Whitney et al., Plant Physiol. 155: 27-35, 2011). SeLSX and SeLSM35 plants did not survive on soil at the normal atmospheric CO₂ concentration of -400 p.p.m., but were able to grow in CO₂-enriched (9,000 p.p.m.) air at a rate slower than the wild-type plant. Both transgenic plants have normal appearance (FIG. 5). Previous efforts to engineer tobacco Rubisco demonstrated that the growth rate and photosynthetic properties of transplastomic plants are generally consistent with the expression levels and catalytic properties of the recombinant Rubisco (Whitney et al., Plant Physiol. 155: 27-35, 2011; Whitney et al., Proc. Natl Acad. Sci. USA 98: 14738-14743, 2001). We believe it is also the case in our transplastomic plants. Our preliminary analyses to quantify the Rubisco content using the CABP (2-carboxy-D-arabinitol-1,5-bisphosphate) binding method indicate that the Rubisco concentrations in the two chloroplast transformants are approximately 12-18% of that in the wild-type plant (Table 1) (Yokota et al., Plant Physiol. 77: 735-739, 1985). Table 1 shows Rubisco, total soluble protein and chlorophyll content of the wild-type and transformed homoplastomic tobacco leaves of similar size, development and canopy position. In addition, the lower levels of total soluble proteins and chlorophyll concentrations probably contribute to the observed slow growth of the two chloroplast transformants (Table 1).

TABLE 1

| | Rubisco | Total soluble protein | Chlorophyll a & b |
|---|---|---|---|
| Sample | (g/m²) | (g/m²) | (g/m²) |
| Wild-type | 0.91 ± 0.09 | 3.74 ± 0.06 | 0.32 ± 0.02 |
| SeLSX | 0.11 ± 0.01 | 1.85 ± 0.02 | 0.21 ± 0.01 |
| SeLSM35 | 0.16 ± 0.01 | 1.46 ± 0.09 | 0.18 ± 0.01 |

To obtain the results shown in Table 1, wild-type plants were grown in air and the transformants in air supplemented with 0.9% (v/v) CO₂. Fresh 4 cm² leaf samples were homogenized in (1 ml) ice-cold extraction buffer. The crude homogenate was used for determination of chlorophyll and Rubisco content. The total soluble protein was determined by the Bradford method following extract clarification (13,200 g, 5 min, 4° C.). Values are means ±standard deviation from 3 different leaves per sample.

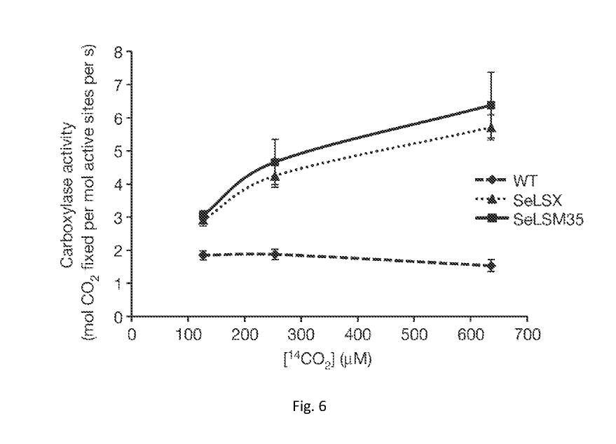

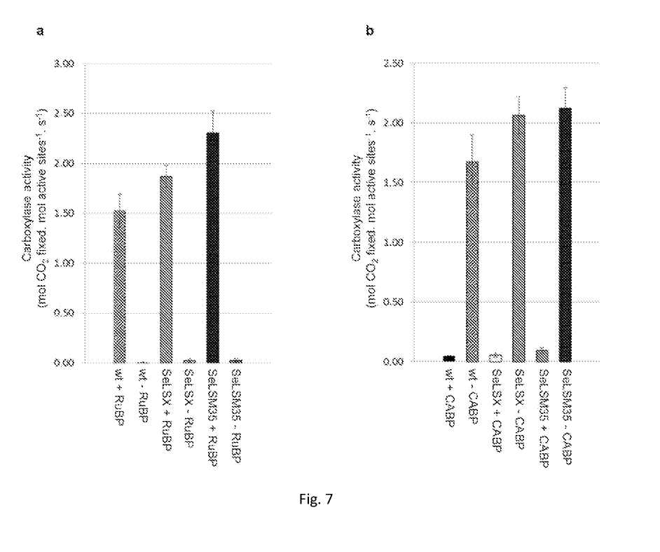

The fact that both transgenic lines could grow autotrophically indicated that active cyanobacterial Rubisco has assembled. We measured the carboxylase activities of the cyanobacterial Rubisco in the leaf homogenates at room temperature using ribulose bisphosphate (RuBP) and several concentrations of radio labelled sodium bicarbonate(NaH¹⁴CO₃). The assays were performed in the presence of 10 mM, 20 mM and 50 mM NaH¹⁴CO₃, which at pH 8.0 would generate dissolved CO₂ concentrations of approximately 125 μM, 250 μM and 640 μM, respectively. The carboxylase activity of Rubisco in the tobacco control did not increase upon increasing the CO₂ concentration, confirming that the native enzyme was already saturated at 125 μM of dissolved CO₂ (FIG. 6). In contrast, cyanobacterial Rubisco displayed greater carboxylase activity at higher CO₂ concentrations, with a rate of catalysis which exceeded that of the tobacco enzyme at each CO₂ concentration. Our measured kinetic values are consistent with the reported rate and Michaelis constants for CO₂ (˜3 s⁻¹ and 10.7 μM for tobacco and ˜12 s⁻¹ and 200 μM for the enzyme in Synechococcus PCC6301, respectively) (Whitney et al., Plant Physiol. 155: 27-35, 2011; Mueller-Cajar et al., Biochem. J. 414: 205-214, 2008). We confirmed that the carboxylase activities detected in our samples were specific to Rubisco, as they were entirely dependent on the presence of RuBP and were inhibited by CABP25 (FIG. 7). The high carboxylase activities detected in the transformants are consistent with the absence of interference by tobacco SSU in the assembly of bona fide cyanobacterial Rubisco in the chloroplasts. Furthermore, both transgenic lines exhibited high Rubisco activities despite differences in its intra-organellar organization.

We included RbcX in one of our chloroplast transformation vectors because it has been shown to enhance the assembly of the LSU core complex before formation of the final hexadecameric complex (Saschenbrecker et al., Cell 129:1189-1200, 2007). However, Se7942 lacking RbcX suffered no defect in growth rate or Rubisco activity (Emlyn-Jones et al., Plant Cell Physiol. 47: 1630-1640, 2006). As line SeLSM35 lacks RbcX but has active Rubisco, evidently Se-RbcX is not essential for the assembly of functional cyanobacterial Rubisco in chloroplasts. CcmM35, through its SSU-like domains, might assist in the assembly of cyanobacterial Rubisco in SeLSM35 in the absence of RbcX.

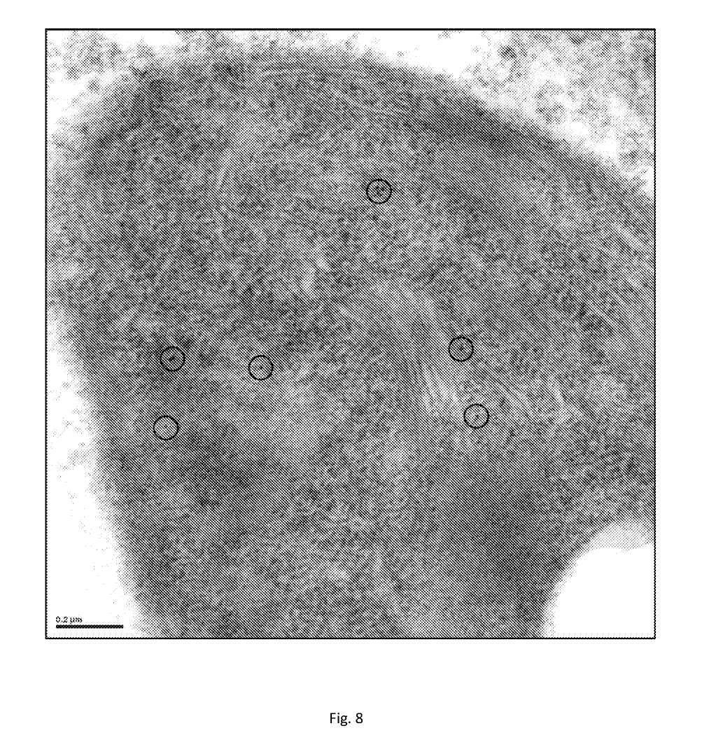

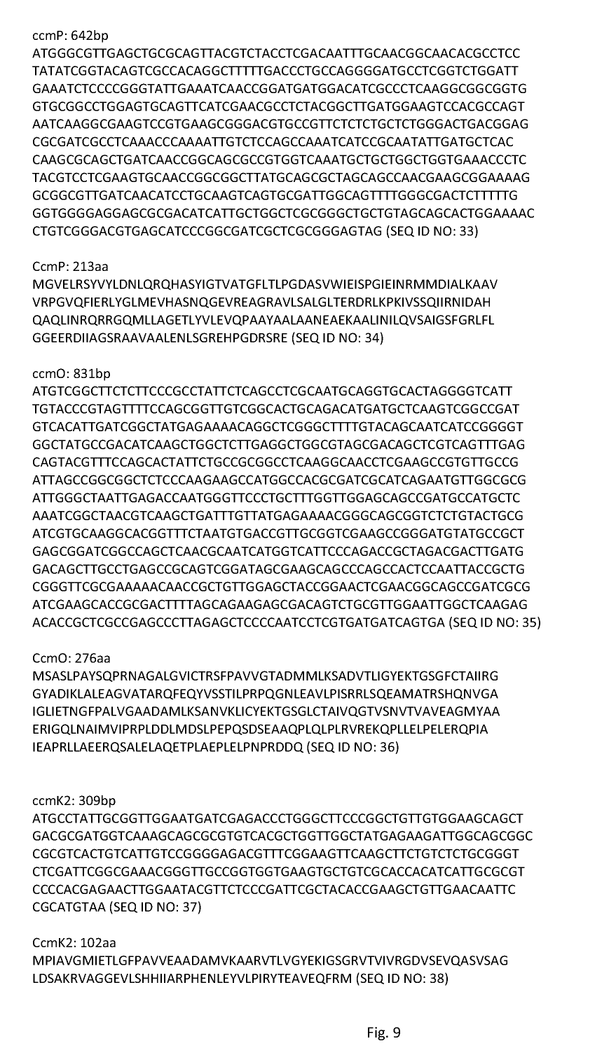

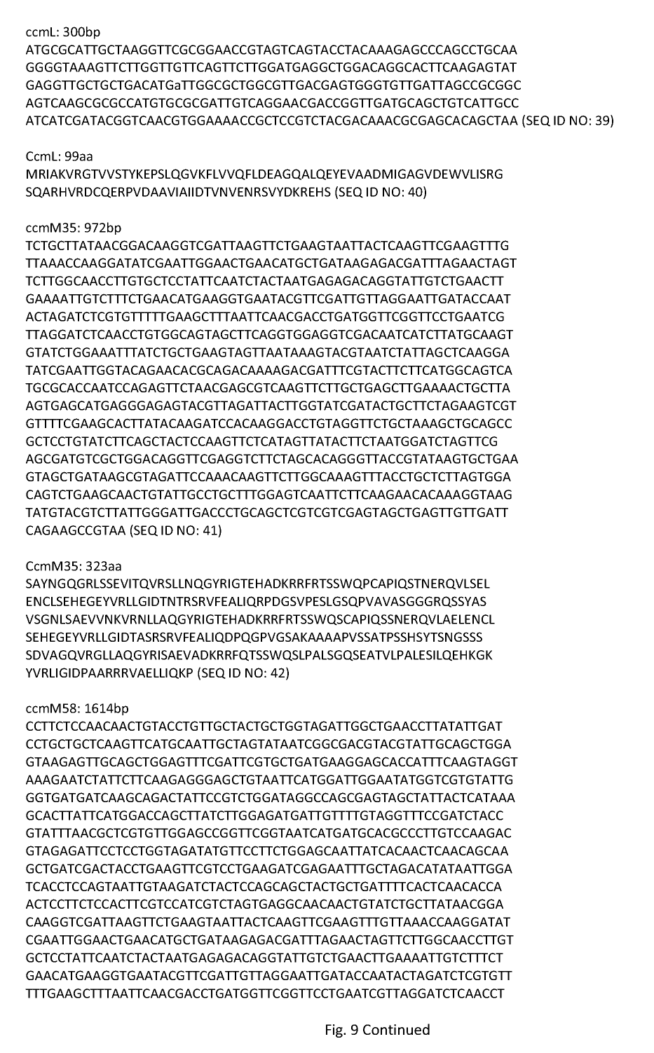

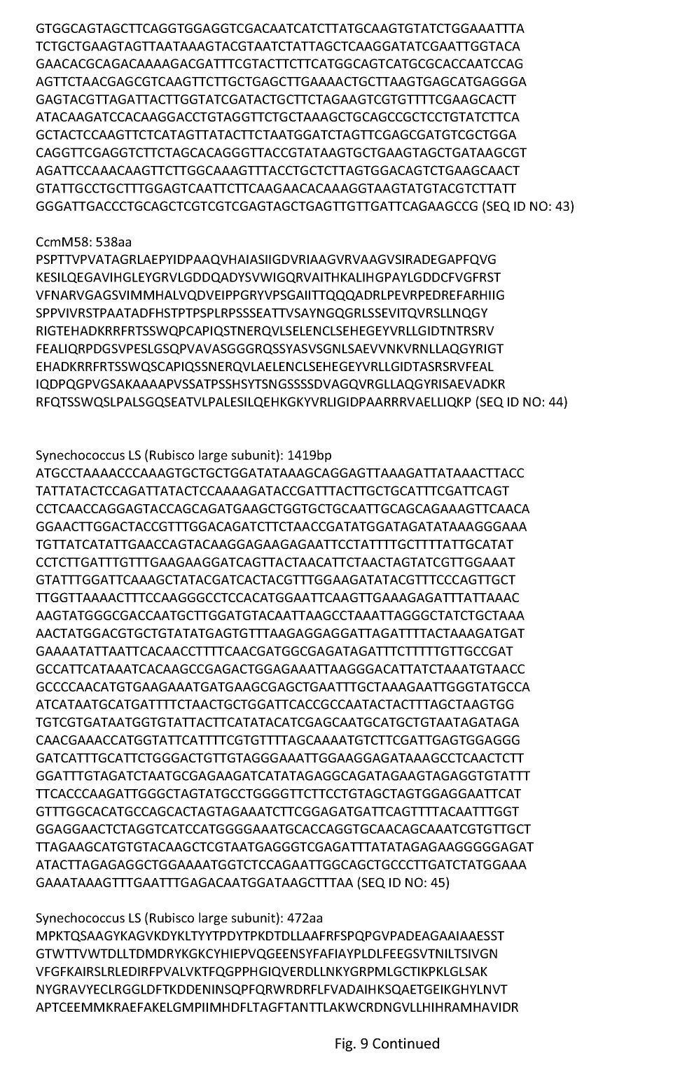

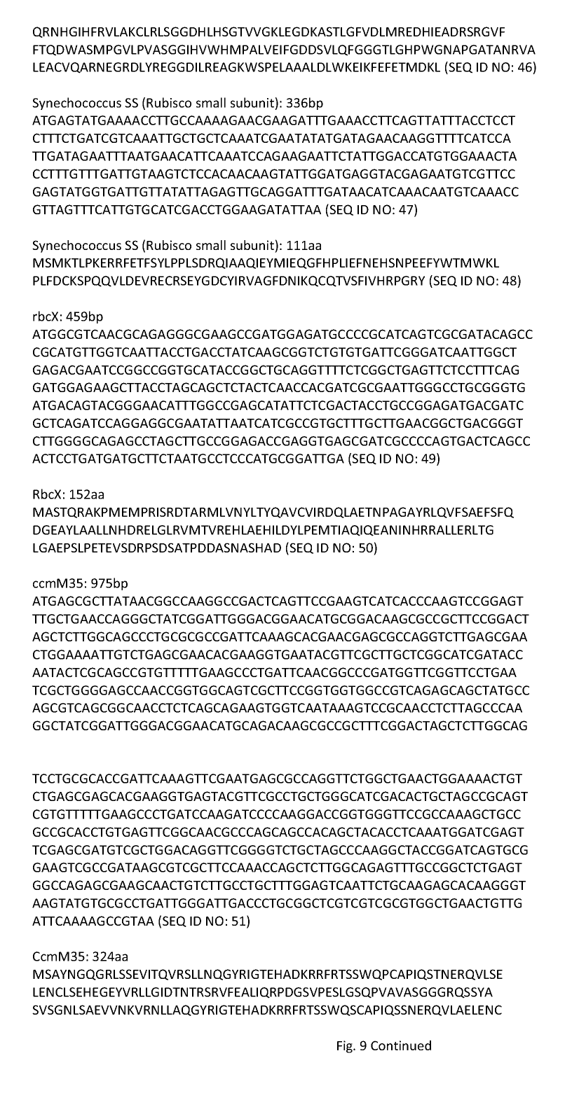

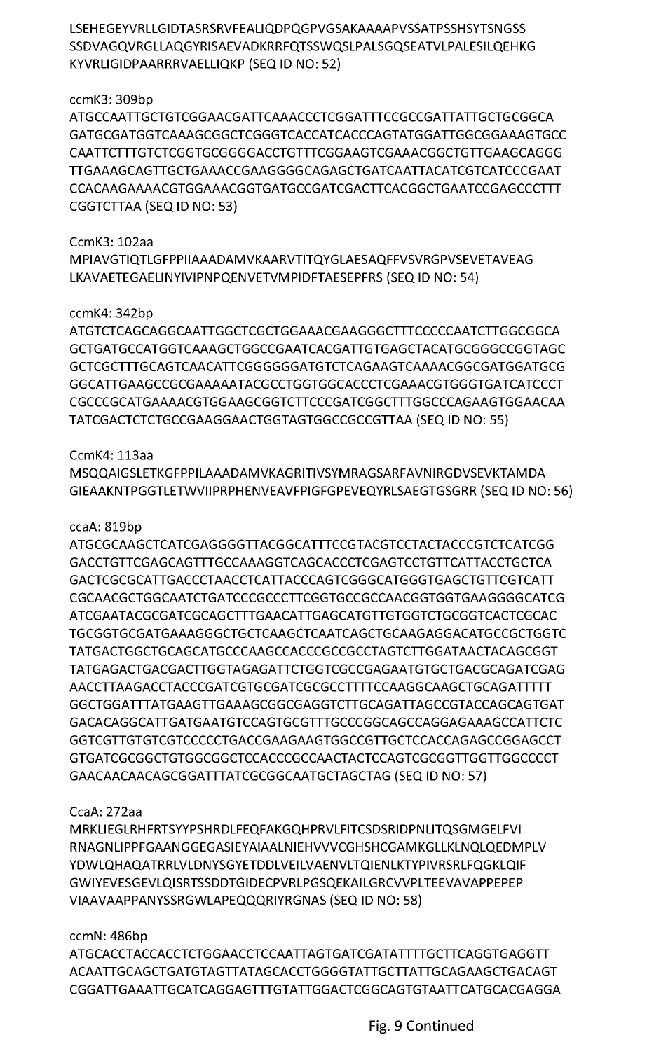

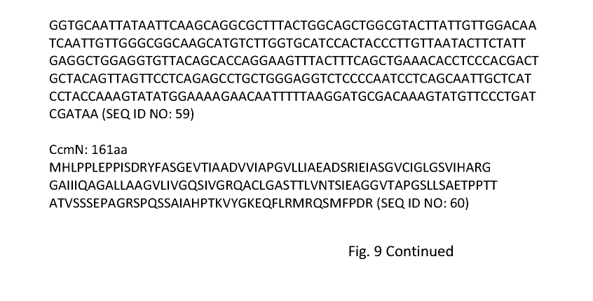

In addition, to test whether cyanobacterial LSU and SSU can assemble into a functional enzyme within higher plant chloroplasts, we generated a third transplastomic tobacco lines, named SeLS to express the two Rubisco subunits without rbcX or M35. The vector used to express SeLS without rbcX or M35 is shown in the Material and Methods (below). SeLS plants, like SeLSX and SeLSM35 plants did not survive on soil at the normal atmospheric CO₂ concentration of ˜400 p.p.m., but were able to grow in CO₂-enriched (9,000 p.p.m.) air at a rate slower than the wild-type plant. Carboxylases activities of SeLS were equivalent to those found in SeLSX plants. The distribution of Rubisco in the chloroplast stroma of SeLS plants is similar to that in SeLSX plants (FIG. 8). And like SeLSX and SeLSM35 plants, SeLS plants have normal appearance.

Materials and Methods

The above-described results in Example 1 were performed using the following materials and methods.

Construction of the transformation vectors. The Se-rbcL and Se-rbcS genes with codons optimized for chloroplast translation system were designed by Muhammad Waqar Hameed and synthesized by Bioneer. Table 2 contains the primers ordered from Integrated DNA Technologies and used in this work.

The amplifications of DNA molecules were carried out with Phusion High-Fidelity DNA polymerase (Thermo Scientific). The restriction enzymes and T4DNA ligase were also purchased from Thermo Scientific.

TABLE 2
Oligonucleotides used in the construction of choloroplast transformation vectors, DNA blot
analyses of the tobacco chloroplast rbcL locus and RT-PCR analyses of the tobacco
chloroplast rbcL gene and transgenes introduced in the transplastomic lines.

| Primers | Nucleotide sequences |
|---|---|
| F10LrbcLfor | CATGAGTTGTAGGGAGGGATTTATGCCCTAAAACCCAAAGTGCTG (SEQ ID NO: 1) |
| 4RErbcLrev | ATAACGCGTCTGCAGGGCAGGCGGCCGCCGCGCGCGTTAAAGCTTATCCATTGTCTCAAA (SEQ ID NO: 2) |
| F1for | GGCCCCCACTATCTCGACCTTGAACTACC (SEQ ID NO: 3) |
| F1for2 | AGCTCGGGCCCCAAATAATGATT (SEQ ID NO: 4) |
| F1rev | AAATCCCTCCCTACAACTCATG (SEQ ID NO: 5) |
| F2for | ATGCCTGCAGATGCAGGTCGACCATATGAAACAGTAGACATTAGCAGATAAATTAG (SEQ ID NO: 6) |
| F2rev | TCCAACGCGTTGGAAATAATCAACATTACTGCAACTAGAATTG (SEQ ID NO: 7) |
| SMOfor | CTATTGCTCCTTTCTTTTTCTGCAG (SEQ ID NO: 8) |
| SMOrev | ATGCCTGCAGGATAACTTCGTATAGCATACATTATACG (SEQ ID NO: 9) |
| TrbcLfor | AGATCGCGCGCGAAACAGTAGACATTAGCAGATAAATTAG (SEQ ID NO: 10) |
| TrbcLrev | AGATGGGCCCTTCAAATCTTGTATATCTAGGTAAGTATATAC (SEQ ID NO: 11) |
| IEESDrev | GTATATCTCCTTCTTGAGATCTGTTGACTTTGTATCCATTCCGTTGTAAATAAATGATC (SEQ ID NO: 12) |
| IEESD18rev | CCCATATGTATATCCTTCTCCCTATGTATATCTCCTTCTTGAGATCTGTTGAC (SEQ ID NO: 13) |
| SD19rev2 | CATGGGTATATCTCCTTCTCCCATATGTATATCTCCTTCTCCCATATGTA (SEQ ID NO: 14) |
| TpelDAtfor | AGATGGGCCCCACGCGTCGCGCGCGTTCAATTATTCAATTGTAAAATAAACGACG (SEQ ID NO: 15) |
| TpelDAtrev | CCATTCCGTTGTAAATAAATGATCTTAACCCATTTTAATTAATTAATTAAATTAATTAG (SEQ ID NO: 16) |
| TpsbAAtfor | AGATGGGCCCACGCGTCGCGCGCGTTCGTTAGTGTTAGTCAGATCTAG (SEQ ID NO: 17) |
| TpsbAAtrev | CCATTCCGTTGTAAATAAATGATCTTAAATATGATACTCTATAAAAATTTGCTC (SEQ ID NO: 18) |
| Trps16Atfor | AGATGGGCCCACGCGTCGCGCGCGAGTCTTACTAAAACGAATGAAATTAATG (SEQ ID NO: 19) |
| Trps16Atrev | CCATTCCGTTGTAAATAAATGATCTTACAAAATAAATATGATGGAAGTGAAAGAG (SEQ ID NO: 20) |
| rbcSfor | GTCAACAGATCTCAAGAAGGAGATATACCCATGAGTATGAAAACCCTTGCCAAAAG (SEQ ID NO: 21) |
| rbcSrev | AGATGCGGCCGCACGCGTTTAATATCTTCCAGGTCGATGCAC (SEQ ID NO: 22) |
| rbcXfor: | GTCAACAGATCTCAAGAAGGAGATATACCCATGGCGTCAACGCAGAGG (SEQ ID NO: 23) |
| rbcXrev: | AGATGCGGCCGCACGCGTCAATCCGCATGGGAGGCATTAG (SEQ ID NO: 24) |
| M35for: | GTCAACAGATCTCAAGAAGGAGATATACCCATGAGCGCCTTATAACGGCCAAGG (SEQ ID NO: 25) |
| M35rev | AGATGCGGCCGCACGCGTTTACGGCTTTTGAATCAACAGTTCAGC (SEQ ID NO: 26) |
| aadAfor: | ATGGCTCGTGAAGCGGTTATCG (SEQ ID NO: 27) |
| aadArev: | TTATTTGCCAACTACCTTAGTGATCTCG (SEQ ID NO: 28) |
| SSprbfor: | ACCATGCAATTGAACCGATTCAATTG (SEQ ID NO: 29) |
| SSprbrev: | TGTATACTCTTTCATATATATAGCGCCA (SEQ ID NO: 30) |
| Nt-rbcLfor: | ATGTCACCACAAACAGACTAAAG (SEQ ID NO: 31) |
| Nr-rbcLrev: | TTACTTATCCAAAACGTCCCACTGCTG (SEQ ID NO: 32) |

The two tobacco chloroplast genomic loci (F1 and F2) immediately flanking the rbcL gene (base pairs 56620-57599 and 59034-60033 of NCBI Reference Sequence: NC_001879.2) were amplified from the DNA extracted from tobacco plants using the primer pairs F1for-F1 rev and F2for-F2rev respectively and cloned into pCR8/GW/TOPO TA vector (Life Technologies) adding PstI and MluI restriction sites at the 59 and 39 end of F2, respectively. The Se-rbcL gene was amplified from pGEMTeasy-Se-rbcL with F1OLrbcLfor and 4RErbcLrev primers adding an overlap to the 3′ end of F1 at the 5′ end of Se-rbcL and four restriction sites, MauBI, NotI, PstI and MluI, at the 3′ end of Se-rbcL. This amplified Se-rbcL gene was designed to replace the tobacco rbcL in frame and allow the synthetic expansion of the operon. F1for2 and F1rev primers were used to amplify F1 from its pCR8 vector and the resulting product was then joined with the Se-rbcL amplicon by the overlap extension PCR procedure. The F1-Se-rbcL segment was then digested with ApaI and MluI restriction enzymes and ligated in top GEM-Teasy-Se-rbcL template treated with the same two enzymes to obtain the pGEM-F1-rbcL vector. F2 was digested out of its pCR8 vector with PstI and MluI enzymes and ligated into the similarly disgested pGEMF1-rbcL to yield the pGEM-F1-rbcL-F2 vector. The selectable marker operon (SMO) containing LoxP-PpsbA-aadA-Trps16-LoxP was amplified from a previously reported chloroplast transformation vector, pTetCBgIC (Gray et al., Plant Mol. Biol. 76:345-355, 2011), with SMOfor and SMOrev primers, digested with PstI and ligated in forward orientation to the PstI digested pGEM-F1-rbcL-F2 vector to obtain the pGEM-F1-rbcL-SMO-F2 vector. The rbcL terminator (TrbcL) was amplified from the tobacco DNA with TrbcLfor and TrbcLrev primers, digested with MauBI and Bsp120I enzymes and ligated between the MauBI and NotI sites of the pGEM-F1-rbcL-SMO-F2 vector to obtain the pCT-rbcL vector, which is ready to replace the tobacco rbcL with Se-rbcL and the SMO by the chloroplast transformation procedure. The Se-rbcL operon driven by the native rbcL promoter in pCT-rbcL was then expanded at the MauBI site with Se-rbcS, Se-rbcX and Se-ccmM35 as follows.

Three terminators from the Arabidopsis thaliana (At) chloroplast genome, TpetD(At), TpsbA(At) and Trps16(At), were amplified with their respective primer pairs, TpetDAtfor-TpetDAtrev, TpsbAAtfor-TpsbAAtrev and Trps16Atfor-Trps16Atrev, adding an overlap to the intercistronic expression element (IEE) at the 3′ end and two restriction sites, MluI and MauBI at the 5′ end of each terminator. Each terminator was extended at the 3′ end by IEE-s.d. or IEE-SD18 fragment with primers IEESDrev or IEESD18rev-SD18rev2 respectively, resulting in the four intergenic regions, IG1, 1G2, 1G3, and 1G4 in FIG. 1a. The Se-rbcX and SeccmM35 genes were amplified from the genomic DNA extracted from Se7942 using the primer pairs rbcXfor-rbcXrev and M35for-M35rev respectively, adding an overlap to the IEE-s.d. fragment at the 5′ end and a MluI site at the 3′ end of each gene. Similarly, Se-rbcS was amplified from pGEM-Teasy-Se-rbcL using the primer pair rbcS for-rbcSrev.Then, IG1-rbcS, IG2-rbcX, IG3-rbcS and 1G4-ccmM35 fragments were similarly generated by joining each intergenic fragment with the corresponding gene using the overlap extension PCR procedure. The MluI-digested 1G2-rbcX and 1G4-ccmM35 modules were each inserted into the MauBI site of the pCT-rbcL to obtain pCT-rbcL-rbcX and pCT-rbcL-ccmM35, respectively. Then the MluI-digested IG1-rbcS and 1G3-rbcS modules were each inserted into the MauBI site of pCT-rbcL-rbcX and pCT-rbcL-ccmM35 to obtain pCT-LSX and pCT-LSM vectors, respectively, which were used in the following chloroplast transformation procedure to replace the native rbcL gene with the cyanobacterial genes.

Generation of transplastomic tobacco plants. We used the Biolistic PDS-1000/He Particle Delivery System (Bio-Rad Laboratories) and a tissue-culture based selection method (Maliga et al., Methods Mol. Biol. 1132: 147-163, 2014). Two-week-old tobacco (Nicotiana tabacum cv. Samsun) seedlings germinated in sterile MS agar medium were bombarded with 0.6 μm gold particles carrying the appropriate chloroplast transformation vector. Two days later, the leaves were cut in half and put on RMOP agar plates containing 500 mg1⁻¹ of spectinomycin and incubated for 4-6 weeks at 23° C. with 14 h of light per day. The shoots arising from this medium were cut into small pieces of about 5 mm² and subjected to the second round of regeneration in the same RMOP medium for about 4-6weeks. The shoots from the second selection round were then transferred to MS agar medium containing 500 mg/l of spectinomycin for rooting and then to soil for growth in a greenhouse chamber with elevated atmospheric CO₂

DNA blot analyses of the rbcL locus of the chloroplast genome. We synthesized the digoxigenin(DIG)-sUTP-labelled DNA probe (56907-57411 of NCBI Reference Sequence: NC_001879.2) with PCR DIG Probe Synthesis Kit by Roche and SBprbfor-SBprbrev primer pair. The total DNA from leaf tissues were extracted with a standard CTAB-based procedure. The leaf tissues frozen in liquid nitrogen were finely ground in Eppendorf tubes in 600 μl of 2× CTAB buffer (2% hexadecyltrimethyl ammonium bromide, 1.4M sodium chloride, 20 mM EDTA, 100 mM Tris pH 8.0, 0.2% beta-mercaptoethanol) and incubated at 65° C. for 1 h. The DNA was extracted with 600 μl of chloroform containing 4% isopropanol. The DNA present in the upper layer transferred to a clean tube was precipitated with 0.8 volume of isopropanol at −70° C. for 1 h and pelleted with a microcentrifuge. The DNA pellet was washed with 200 μl of 70% ethanol and air-dried before it was dissolved in 100 μl of double-distilled water. After quality and concentration of the DNA samples were determined by a NanoDrop method, 1 μg of each DNA sample was digested by Ndel and Nhel restriction enzymes, and the digested fragments were separated on a 1% agarose gel. The DNA pieces in the gel were depurinated, denatured and then transferred and cross-linked to a nylon membrane according to the manufacturer's protocols. The DNA samples on the membrane blot were hybridized with the DIG-labelled probe, which was then detected with anti-digoxigenin alkaline phosphatase antibody using CDP-star chemiluminescent substrate (Roche) according to the manufacturer's specifications.

Analyses of the transcripts by RT-PCR. Total RNA was extracted from each leaf tissue sample with a standard TRIzol procedure. The leaf tissues frozen with liquid nitrogen were ground in 800 μl of trizol and incubated at 22° C. for 5 min. After the insoluble pieces were removed by centrifugation, 160 μl of chloroform was added to the supernatant, mixed vigorously for 15 s and incubated at 22° C. for 3 min. The two aqueous phases were separated in a centrifuged at 4° C. for 15 min and the upper layer transferred to a new tube was mixed with 500 μl of isopropanol. The sample was incubated at 22° C. for 10 min and centrifuged at 4° C. for 10 min. The pellet was resuspended in 800 μl of 75% ethanol and centrifuged again at 4° C. for 10 min. The pellet was air-dried and resuspended in 50 μl of molecular biology grade water. The RNA samples were treated with DNase using Ambion DNA-free kit (Life Technologies) and the cDNA for each gene was generated with its corresponding reverse primer using Sensiscript Reverse Transcription kit (Qiagen) according to the manufacturer's protocols. The cDNA samples were amplified with the PCR master mix (Bioline) and analysed in a 1% agarose gel.

SDS page, immunoblot and determination of CcmM35/Rubisco content. The crude leaf homogenates used in the carboxylase activity measurements were separated by SDS-PAGE using 4-20% polyacrylamide gradient gels (ThermoScientific, UK). For each sample, the same amount of protein, as determined by Bradford assay, was loaded onto the gel. After electrophoresis, the resolved proteins were transferred to a nitrocellulose membrane (Hybond-C Extra from GE Healthcare Life Sciences) using a western blot apparatus. The nitrocellulose membranes were immunoblotted using one of four primary polyclonal antibodies raised against: cyanobacterial (SePCC6301) Rubisco; tobaccoRubisco; the small subunit of tobacco Rubisco; and CcmM from Se PCC7942. The primary polyclonal antibody to detect CcmM35 was generated in rabbit with His-tagged CcmM58 protein purified from E. coli (Cambridge Research Biochemicals, UK) and used at a dilution of 1:500 in the immunoblots and from 1:500 to 1:2,000 for immunogold labelling, and was highly specific for CcmM (FIG. 2a). The primary antibodies were visualized by means of a secondary goat anti-rabbit peroxidase-conjugated antibody (Sigma). The absolute and relative content of Synechococcus Rubisco and CcmM35 in SeLSM35 leaves were determined using immunoblots with antibodies against CcmM and cyanobacterial Rubisco. The amounts of Rubisco and CcmM35 present in crude leaf homogenates were estimated by comparison with authentic protein standards (purified CcmM35 and cyanobacterial Rubisco). Amounts of CcmM35 and cyanobacterial Rubisco (μmol m⁻²) were the mean ±standard deviation for duplicate determinations. The band intensities were obtained using ImageJ software (NIH, USA) and the standard curves using Microsoft Excel.

Purification of cyanobacterial Rubisco and CcmM35 proteins. Synechococcus Rubisco was expressed in E. coli BL21 (DE3) cells using the vector pAn92 as previously described (Bainbridge et al., J. Exp. Bot. 46: 1269-1276, 1995). This material was harvested by centrifugation and resuspended in buffer containing 0.1M Bicine-NaOH pH 8.0, 20 mM MgCl₂, 50 mM NaHCO₃, 100 mM PMSF and bacterial protease inhibitor cocktail (Sigma). All steps in the purification were conducted at 0° C. The harvested cells were sonicated and cell debris removed by centrifugation (17,400 g, 20min, 4° C.). PEG-4000 and MgCl₂were added to the supernatant, giving final concentrations of 20% (w/v) and 20 mM, respectively. After 30min at 0° C., the precipitated Rubisco was sedimented by centrifugation (17,400 g, 20min, 4° C.) and the pellet resuspended in 25 mM triethanolamine (pH 7.8, HCl), 5 mM MgCl₂, 0.5 mM EDTA, 1 mM ε-aminocaproic acid, 1 mM benzamidine, 12.5% (v/v) glycerol, 2 mM DTT and 5 mM NaHCO₃. This material was subjected to anion-exchange chromatography using a 5m1 HiTrap O column (GE-Healthcare) pre-equilibrated with the same buffer. Rubisco was eluted with a 0-600 mM NaCl gradient in the same buffer. Fractions containing the most Rubisco activity (as judged by RuBP-dependent (Zhu et al., Annu. Rev. Plant Biol. 61: 235-261, 2010) CO₂ assimilation) were further purified and desalted by size-exclusion chromatography using a 2032.6 cm diameter column of Sephacryl S-200 HR (GE-Healthcare) pre-equilibrated and developed with (50 mM Bicine-NaOH pH8, 20 mM MgCl₂, 0.2 mM EDTA, 2 mM DTT). The resulting protein peak was concentrated by ultrafiltration using 20 ml capacity/150 kDa cut-off centrifugal concentrators (Thermo Pierce). The PCR-amplified ccmM35 gene from Se PCC7942 was cloned into pCR8/GW/TOPO TA vector (Life Technologies) and subsequently transferred to the Gateway pDEST17 E. coli expression vector (Life Technologies), which utilizes the T7 promoter to express the inserted gene and incorporates a 6XHis tag at the N terminus of the translated protein. The expression vector was transformed into Rosetta (DE3) competent cells, and the protein expression was induced with 0.5 mM IPTG at OD₆₀₀ₙₘ of 0.5. The cells in 0.5 litre LB culture were harvested after 4 h of growth at 37° C. and 250 r.p.m. The cells were resuspended in about 10m1 of ice-cold 50 mM sodium phosphate, 300 mM sodium chloride, 20 mM imidazole at pH 8.0 and broken with sonication. The cell debris were removed by centrifugation and the supernatant was mixed with 2 ml of Ni-NTA resin, which was then washed with 15 ml of the cell suspension buffer in a gravity-flow column and the bound protein was eluted with the buffer containing 200 mM imidazole. The purity of CcmM35 was assessed with SDS-PAGE, and its concentration was determined by the Bradford method.

Cryo-preparation of leaf material and transmission electron microscopy. Leaf material was cryofixed at a rate of 20,000 Kelvins per sec using a high pressure freezer unit (Leica Microsystems EM HPM100). The second step of freeze substitution of cryofixed samples was performed in an EMAFS unit (Leica Microsystems) at −85° C. for 48 h in 0.5% uranyl acetate in dry acetone. The samples were then infiltrated at low temperature in Lowicryl HM20 resin (Polysciences) and polymerized with a UV lamp (Lin et al., Plant J. 79:1-12, 2014).

For the immunogold labelling, gold grids carrying ultrathin sections (60-90 nm) of leaf tissue embedded in HM20 were treated using different rabbit primary antibodies against: cyanobacterial Rubisco from Se PCC6301; tobacco Rubisco; and CcmM35 (produced by Cambridge Research Biochemicals). A secondary goat polyclonal antibody to rabbit IgG conjugated with 10 nm gold particles (Abcam, UK) was used for the labelling.

Images were obtained using a transmission electron microscope (Jeol 2011 F) operating at 200 kV, equipped with a Gatan Ultrascan CCD camera and a Gatan Dual Vision CCD camera.

Plant material and growing conditions. Both transgenic and wild-type Nicotiana tabacum var. Samsun NN were grown in the same controlled environment chamber with 16 h of fluorescent light (43%) and 8 h dark, at 24° C. during the day and 22° C. during the night. The relative humidity was 70% during the day and 80% during the night. The atmospheric CO₂ concentration was kept constant at 9,000 p.p.m. (air containing 0.9% v/v CO₂).

Quantification of protein, Rubisco, and chlorophyll. Total soluble protein in the leaf homogenates was determined by the standard Bradford method. Rubisco active site concentration in the crude homogenate was determined using the [¹⁴C]-CABP binding assay (Yokota et al., Plant Physiol. 77: 735-739, 1985) or by quantifying LSU band intensity by immunoblotting. Each approach gave very similar results. Chlorophyll concentration was determined spectrophotometrically using unfractionated leaf homogenates (Wintermans et al., Biochim. Biophys. Acta 109: 448-453, 1965).

Carboxylase activity measurements. Leaf discs (1 cm²) were cut and promptly homogenized using an ice-cold pestle and mortar, in the presence of 500 μl of ice cold extraction buffer (50 mM EPPS-NaOH pH 8.0; 10 mM MgCl₂; 1 mM EDTA; 1 mM EGTA; 50 mM 2-mercaptoethanol; 20 mM DTT; 20 mM NaHCO3; 2 mM Na₂HPO₄; Sigma plant protease inhibitor cocktail (diluted 1:100); 1 mM PMSF; 2 mM benzamidine; 5 mM ε-aminocaproic acid). Rubisco carboxylase activity was measured immediately in 500 μl of assay buffer containing 100 mM EPPS-NaOH pH 8.0, 20 mM MgCl₂, 0.8 mM RuBP and 10 mM, 20 mM or 50 mM NaH¹⁴CO₃ (18.5 kBq per mol) at room temperature (22° C.). The assay was initiated by the addition of 20 μl of the leaf homogenate, and was quenched after 2, 4, 6 or 10 min, by the addition of 100 μl of 10M formic acid. The samples were oven dried and the acid stable ¹⁴C determined by liquid scintillation counting, following residue rehydration (400 μl H₂O) and the addition of 3.6 ml liquid scintillation cocktail (Ultima Gold, PerkinElmer, UK).

For Rubisco inhibition using the tight binding Rubisco inhibitor, 2-carboxy-D-arabinitol-1,5-bisphosphate (CABP), leaf homogenates were incubated on ice for 15 min in the presence of 50 μM CABP (Parry et al., J. Exp. Bot. 59: 1569-1580, 2008). Residual carboxylase activity (if any) was then measured as described above.

### EXAMPLE 2

In this example, we again show that neither RbcX nor CcmM35 is needed for assembly of active cyanobacterial Rubisco. Furthermore, by altering the gene regulatory sequences on the Rubisco transgenes, cyanobacterial Rubisco expression was enhanced and the transgenic plants grew at near wild-type growth rates in elevated CO₂. We performed detailed kinetic characterization of the enzymes produced with and without the RbcX and CcmM35 cyanobacterial proteins. These transgenic plants exhibit photosynthetic characteristics that confirm the predicted benefits of non-native forms of Rubisco with higher carboxylation rate constants in vascular plants and the potential nitrogen use efficiency that may be gained provided that adequate CO₂ can be concentrated near the enzyme. Indeed, we demonstrate that that cyanobacterial Rubisco assembles as functional enzyme in tobacco chloroplasts without any added cyanobacterial chaperones, and transgenic plants with up to 10-fold less Rubisco protein are able to grow nearly as fast as wild-type in elevated CO₂, demonstrating the potential gain in nitrogen use efficiency.

In this Example, we further studied the transformant named SeLS and generated an additional transformant named SELSYM35. In the transformant named SeLS, as is described in Example 1 and further in this Example, the two cyanobacterial Rubisco subunits were produced without RbcX or CcmM35, whereas in SeLSYM35 line, we fused YFP to the N-terminus of CcmM35 and optimized the codons of the fused yfp-ccmM35 gene for the chloroplast translation system. We demonstrate that altering the terminator sequences leads to increased accumulation of RNA encoding the cyanobacterial rbcS, which is located 3′ to the rbcL gene in our transgene operons. The improved transgene operons resulted in enhanced Rubisco expression and more rapid growth of the transgenic plants which fixed carbon using the cyanobacterial Rubisco. Thus, neither RbcX or CcmM35 are needed for cyanobacterial Rubisco assembly or vigorous growth under elevated CO₂.

Engineering of the Tobacco Chloroplast Genome with Synthetic Cyanobacterial Operons

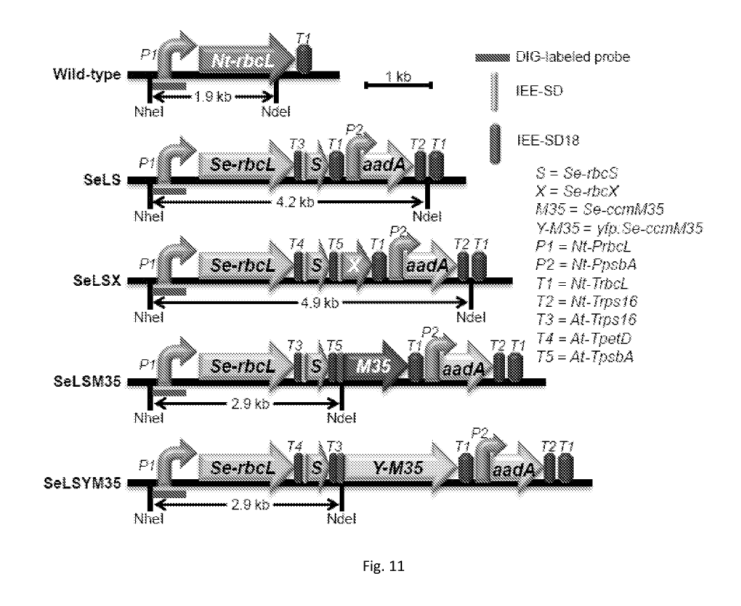

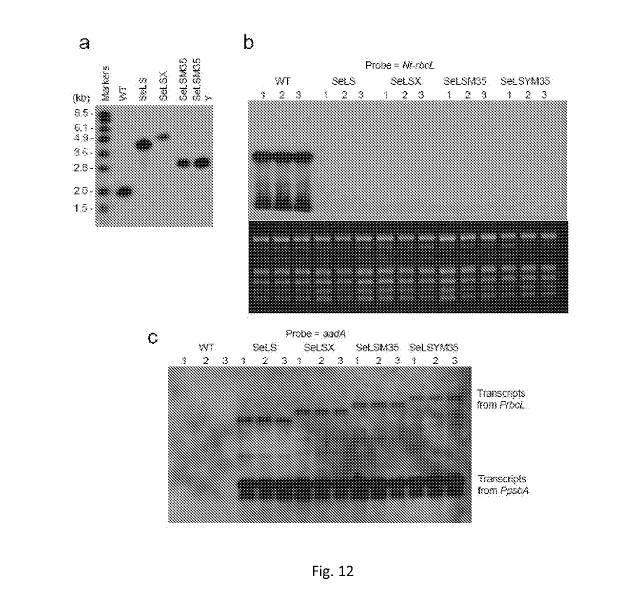

The synthetic operons in SeLS and SeLSYM35 possess similar architecture to the previous ones with a terminator, an intercistronic expression element (IEE) and a Shine-Dalgarno sequence (SD) occupying the intergenic regions (FIG. 11). Such an arrangement has been shown to result in reliable processing of the transcripts for successful translation of downstream genes inside chloroplasts (Lu et al., Proc. Natl. Acad. Sci. USA 110: E623-632, 2013; Lin et al., Nature 513: 547-550, 2014). Three terminators from the Arabidopsis chloroplast and the native rbcL terminator (Nt-TrbcL) were paired with different genes. The ccmM35 gene in SeLSM35 line and the yfp-ccmM35 gene in SeLSYM35 line are each preceded with “SD18” translation signal, which has three tandem Shine-Dalgarno sites for improved translation efficiency (Drechsel et al., Nucleic Acids Res. 39: 1427-1438, 2011). We confirmed the homoplasmy of the chloroplast genomes in the transformants with a DNA blot (FIG. 12a). The complete absence of the native rbcL transcript in the RNA blot also confirmed the successful gene replacement in all four transformants (FIG. 12b).

The use of Different Regulatory Elements in the Transformed Tobacco lines Alters the Expression of Transgenes

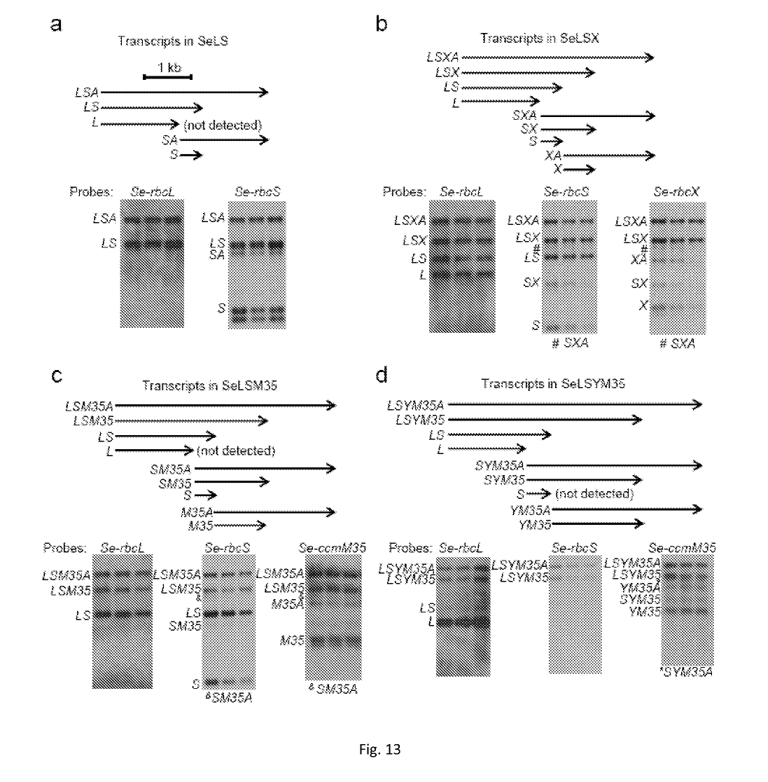

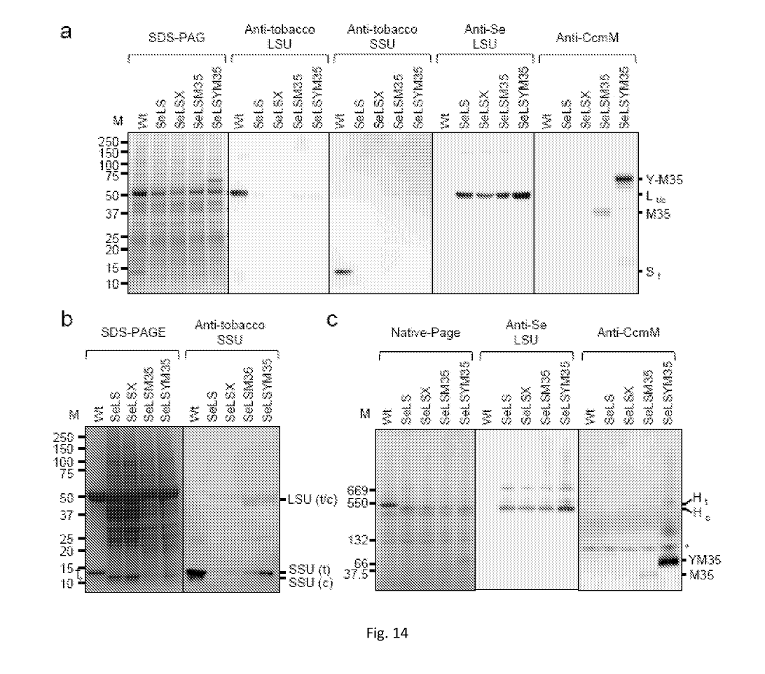

Analyses of the RNA transcripts from the transgene operons show that multigene transcripts are present in all RNA blots, indicating that the IEE sites are only partially processed (FIG. 13). Nevertheless, successful production of Rubisco complexes and CcmM35 proteins indicates that downstream genes in these transcripts are still being translated efficiently (FIG. 14). We found that the transcripts starting at downstream genes such as Se-rbcS and Se-rbcX were significantly less abundant than those starting at the Se-rbcL gene. The aadA transcript produced from the Nt-PpsbA promoter immediately upstream is highly abundant in all four transgenic lines (FIG. 12c).

One function of terminators is to stabilize the transcript upstream. Out of the three terminators from Arabidopsis used in this study, we could not detect any transcript ending with the At-Trps16 terminator, which is used in three of our transgene lines, SeLS, SeLSM35 and SeLSYM35. This observation indicates that the At-Trps16 terminator sequence used in our study does not perform well in stabilizing the upstream transcripts.

SDS-PAGE revealed bands of expected masses for the cyanobacterial and tobacco Rubisco large subunits (LSUs) and small subunits (SSUs), in agreement with published data (Chapman et al., Philos. Trans. R. Soc. Lond. B. Biol. Sci. 313: 367-378, 1986; Long et al., J. Biol. Chem. 282: 29323-29335, 2007) (FIG. 14). The presence of the Rubisco subunits and the two forms of CcmM35 proteins (with or without YFP) was confirmed by immunoblotting SDS-polyacrylamide gels with polyclonal antibodies raised against tobacco LSU, cyanobacterial LSU, tobacco SSU and CcmM. Expression of YFP-CcmM35 is higher in SeLSYM35 compared to the level of CcmM35 in the SeLSM35 line, perhaps due to the codon optimization of the yfp:ccmM35 coding region. Coincidentally, the SeLSYM35 tobacco line also produced the highest amount of cyanobacterial LSU, probably due to the high abundance of the corresponding transcript as well as the stabilizing effect of YFP-CcmM35 in that line.

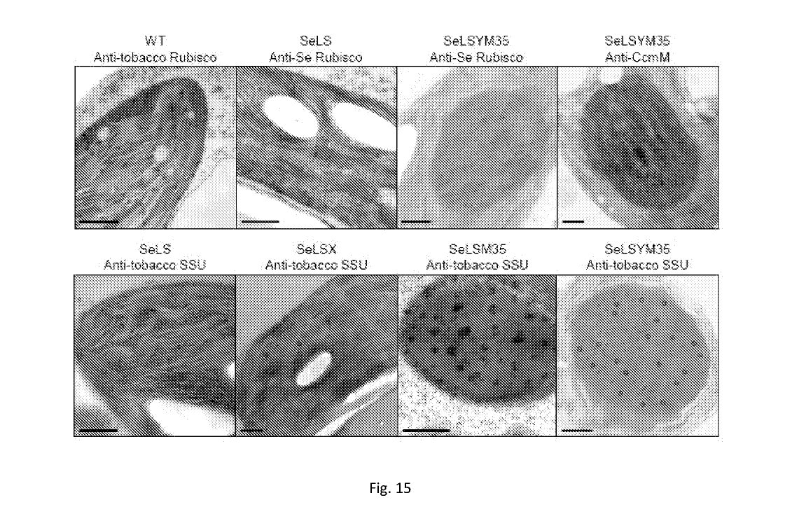

Consistent with previous work, we could not detect tobacco SSU in the total leaf proteins from all four transgenic tobacco lines (FIG. 14a), likely due to its instability in the absence of a compatible LSU (Kanevski et al., Plant Physiol. 119: 133-142, 1999; Whitney et al., Proc. Natl. Acad. Sci. USA 98: 14738-14743, 2001; Lin et al., Nature 513: 547-550, 2014). However, by partial purification of Rubisco following extraction with Triton X-100 and concentration by anion-exchange chromatography, we were able to detect a small amount of tobacco SSU in the Rubisco samples from the transformants, particularly in SeLSM35 and SeLSYM35 (FIG. 14b), indicating that some hybrid Rubisco enzymes containing both the cyanobacterial LSU and the tobacco SSU may have assembled in these transformants. In SeLSM35 and SeLSYM35 lines, the tobacco SSU is seen to be associated with the large complexes formed by the cyanobacterial Rubisco and CcmM35 (FIG. 15).

Expression of CcmM35 Results in Aggregates of Rubisco

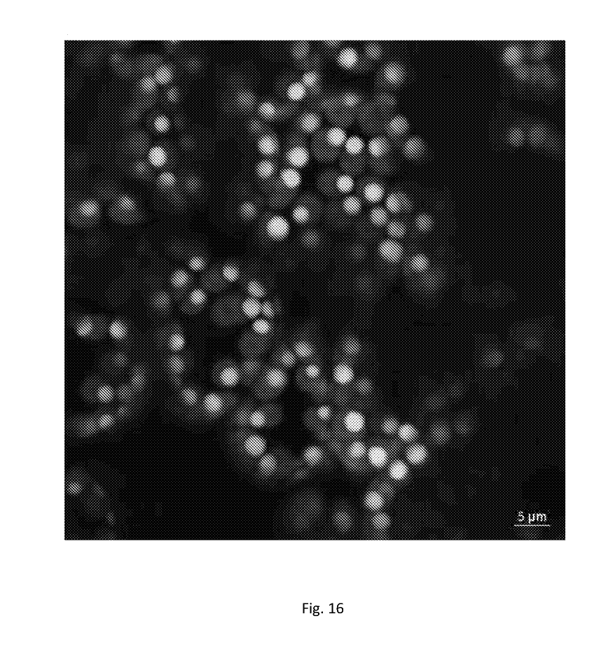

Non-denaturing acrylamide gel electrophoresis revealed bands consistent with the predicted molecular weight of the hexadecameric Rubisco holoenzyme from both tobacco (˜540 kDa) and Se7942 (˜520 kDa) made up of eight LSUs and eight SSUs (FIG. 14c). The composition of the transgenic and wild-type holoenzymes as well as the presence of CcmM35 and YFP-CcmM35 in the SeLSM35 and SeLSYM35 lines were confirmed by immunoblots. Although the formation of such Rubisco holoenzyme is to be expected in SeLS and SeLSX lines, the prevailing models and evidence indicate that CcmM35 may connect a large number of Rubisco complexes into a single and extensive aggregate through interactions between LSUs and SSU-like domains present in CcmM35 (Long et al., Photosynth. Res. 109: 33-45, 2011). We suspect that the treatment of our samples with Triton X-100 prior to gel electrophoresis partially disrupted such interactions, promoting the formation of the hexadecamer complexes observed on the gel. Even with Triton X-100, we were unable to completely solubilize these complexes formed between CcmM35 and Rubisco. The formation of large Rubisco aggregates in the presence of either type of CcmM35 was confirmed by transmission electron microscopy (TEM) with immunogold labelling (FIG. 15) (Lin et al., Nature 513: 547-550, 2014). In the SeLSYM35 line, the fluorescent image of the leaf tissue also displayed large spherical aggregates consistent with those observed by TEM (FIG. 16).

The Transformed Tobacco Plants Display Photosynthetic Performance Consistent with the Kinetic Properties of Cyanobacterial Rubisco

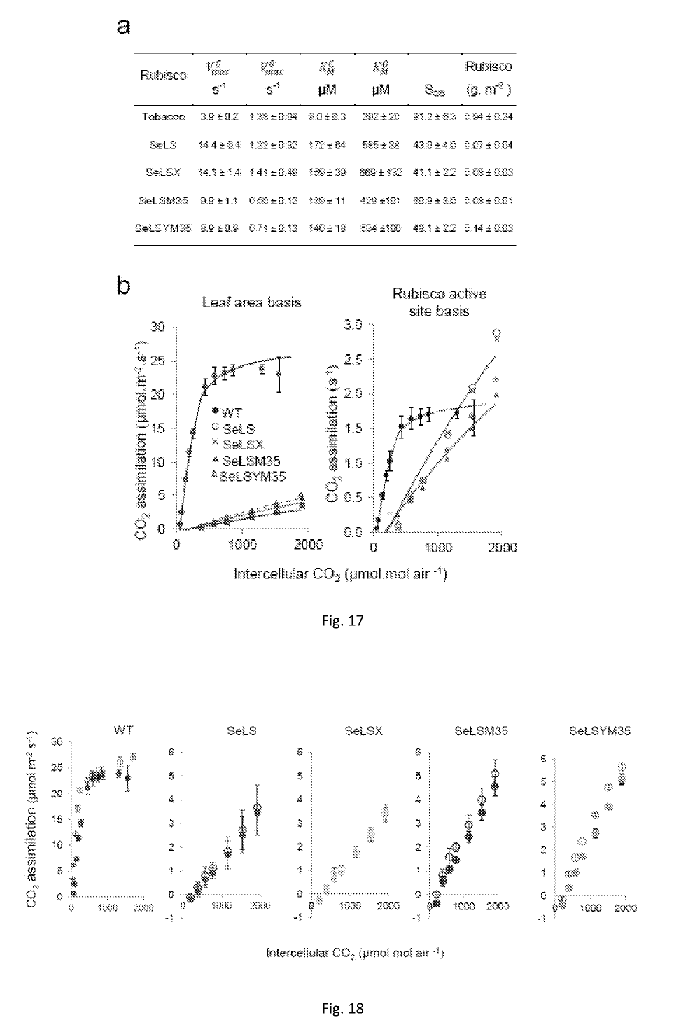

The kinetic parameters of the Rubisco extracted from leaves of SeLS and SeLSX (FIG. 17a) were the same as those reported in the literature for the native enzyme extracted from the cyanobacterium Synechococcus PCC7942 (Vₘₐₓ^{C}=˜14.4 s⁻¹ and K_{M}^{C}=˜169 μM) (Whitehead et al., Plant Physiol. 165: 398-411, 2014). Both the maximum catalytic rates (Vₘₐₓ^{C}) and Michaelis constants (K_{M}^{C}) for the enzymes extracted from SeLSM35 and SeLSYM35 were lower, probably due to the effect of minor amounts of tobacco SSU in the enzymes in these lines. The values for the specificity factors are consistent with published values, as were the kinetic properties of tobacco Rubisco, measured contemporaneously, validating our experimental approach (Whitney et al., Plant Physiol. 121: 579-588, 1999; Whitney et al., Proc. Natl. Acad. Sci. USA 98: 14738-14743, 2001).

We also determined the CO₂ dependence of photosynthesis (A-Ci) for all tobacco lines, both in normal air (FIG. 17b) and with a 10-fold reduction in ambient oxygen that would suppress photorespiration (FIG. 18). Expressed on a leaf area basis, it is clear that wild type tobacco had higher rates of net photosynthesis than any of the transgenic lines, at all intercellular CO₂ concentrations (Ci) (FIG. 17b). Suppression of photorespiration under diminished atmospheric O₂ was greater in the control than in the transgenic lines, as evident from the accompanying stimulation of photosynthesis at non-saturating levels of CO₂ (FIG. 18). When the rate of CO₂ assimilation was expressed relative to the corresponding concentration of Rubisco catalytic sites (FIG. 17b), the contrasting properties of the tobacco and cyanobacterial forms of Rubisco were evident: the tobacco enzyme saturating at 500 μmol intercellular CO₂ mol air⁻¹ with a rate of less than 2 s⁻¹, while turnover by the cyanobacterial counterpart continued to increase linearly across the entire range of CO₂ exceeding the rate of tobacco, although remaining well below the theoretical maximum of 14 s⁻¹ in the absence of O₂ [(Whitehead et al., Plant Physiol. 165: 398-411, 2014) and FIG. 17a] even at the highest level of CO₂. These observations fully support the respective rates and substrate affinity parameters in FIG. 17a, since substitution of these parameters into the biochemical model of leaf photosynthesis of Farquhar et al. (Farquhar et al., Planta 149: 78-90, 1980) generated curves which approximated the experimental data (FIG. 17b).

SeLS and SeLSYM35 Grow Substantially Faster than the SeLSX and SeLSM35 Transformants even in the Absence of Cyanobacterial Assembly Factors

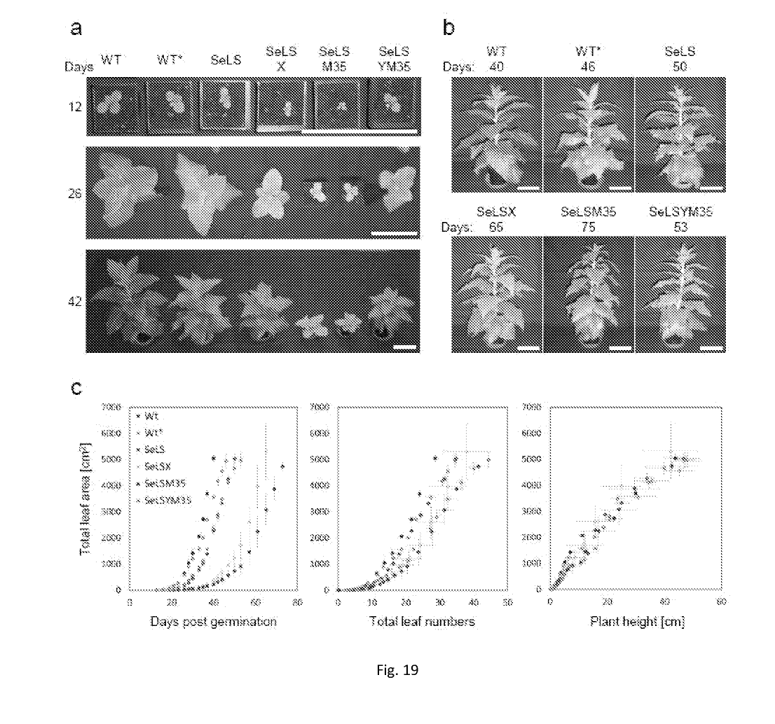

The growth and morphological characteristics of lines expressing Se7942 Rubisco were investigated during growth in air supplemented with 3% (v/v) CO₂. The two new transgenic lines exhibited substantially improved growth compared to the original transgenic lines, with the growth rate of SeLS approaching that of wild-type in 3% CO₂ despite lacking both RbcX and CcmM35 (FIG. 19). The SeLS and SeLSYM35 lines reached the same chosen end-point (immediately preceding anthesis, when the lines had a total leaf area of ˜5,000 cm² per plant) as the controls grown at 3% CO₂ only 4 to 7 days later, whereas the SeLSX and SeLSM35 lines reached the same developmental stage 19 and 27 days later, respectively (FIG. 19b). Acclimation of the wild-type plants to 3% CO₂ (Miller et al., Plant Physiol. 115: 1195-1200, 1997; Schaz et al., AoB Plants 6: 1-16, 2014) delayed them by about 6 days, relative to plants grown at ambient CO₂ (FIG. 19b). Furthermore, the wild-type tobacco plants grown at ambient CO₂ (400 μmol. CO₂ mol air⁻¹) were slightly shorter and showed a lower number of leaves at equivalent values of leaf area (FIG. 19c). The leaf distribution of SeLS tobacco plants was indistinguishable from that of the wild-type plants, whereas the other three transformants had numerous smaller leaves at equivalent values of total leaf area (FIG. 19c). However, all transformants expressing cyanobacterial Rubisco displayed total leaf areas similar to those of the wild-type plants at comparable heights (FIG. 19c).

The SeLS Transformant Displayed Dramatically Higher Efficiency in Rubisco Investment

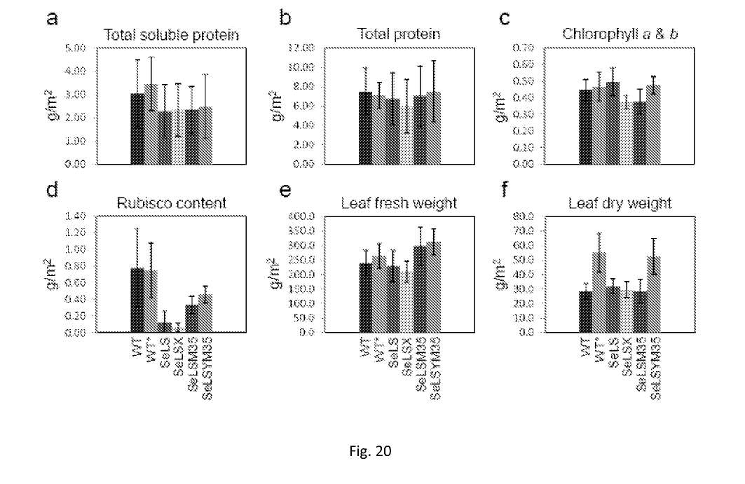

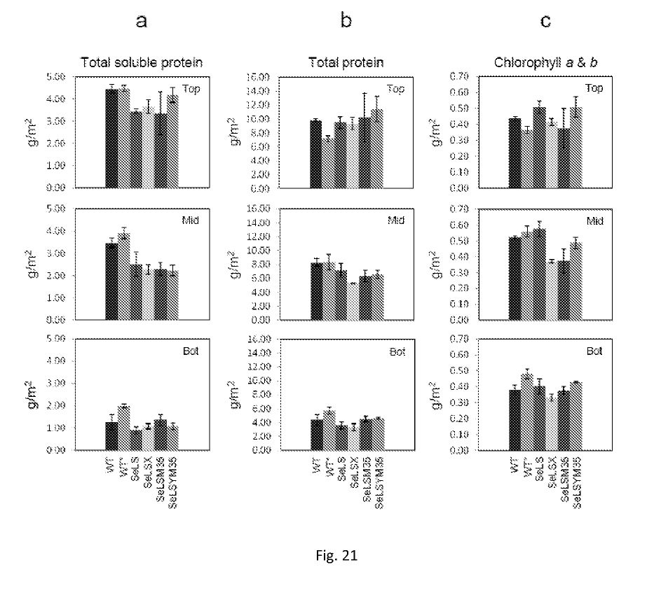

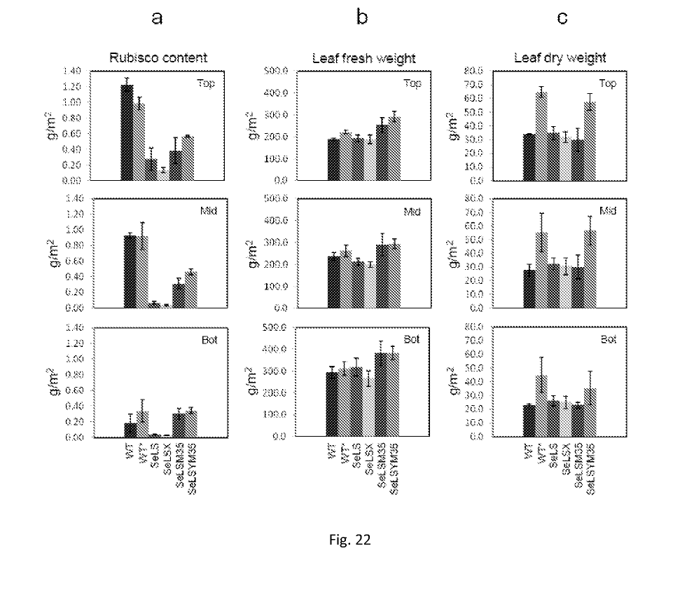

We determined leaf total protein, soluble protein, chlorophyll, Rubisco, fresh and dry mass, from plants at a similar developmental stage (total leaf area ˜5,000 cm² plant⁻¹, pre-anthesis) (FIG. 20). These constituents were also measured in leaves at three different positions on the tobacco shoots (youngest fully expanded (top), oldest non senescent (bottom) and intermediate leaves (middle)) (FIGS. 21 and 22). In general, the amounts of soluble protein in the four transgenic lines were lower than those in wild-type tobacco controls. The amounts of total protein in the two lines expressing CcmM35 (SeLSM35 and SeLSYM35) were similar to those in the controls, whereas they were lower in SeLS and SeLSX. The total chlorophyll contents were higher in SeLS and SeLSYM35, which also grew faster than the other transformants. More importantly, all four tobacco transformants, particularly SeLS and SeLSX, produced substantially less cyanobacterial Rubisco than the wild-type Rubisco in the control plants (FIG. 20d). Remarkably, the SeLS plants with up to 10-fold less Rubisco (FIG. 17a, FIG. 20d) were able to achieve growth rates approaching those of the wild-type plants, indicating the potential benefits of utilizing an inherently faster Rubisco.

As expected, the amount of protein (including Rubisco) declined from the youngest fully expanded to the oldest non senescent leaves (FIGS. 21 and 22). SeLSYM35 and SeLSM35 had higher Rubisco contents than SeLS and SeLSX, and the difference became more pronounced in the intermediate and oldest non senescent leaves (FIG. 17a). This suggests that association with CcmM35 can inhibit the degradation of cyanobacterial Rubisco. The fresh weights per unit leaf area in SeLSM35 and SeLSYM35 were higher than even the control plants (FIG. 17b). Relative to SeLSM35, the faster-growing SeLSYM35 exhibited greater dry weight, which was close to the value measured for the wild-type tobacco grown at the same CO₂ concentration (FIG. 17c).

### SUMMARY

In Example 1 and Example 2, SeLS, expressing only the two cyanobacterial Rubisco subunits without RbcX and CcmM35 was studied. Rubisco extracted from the SeLS tobacco plants was found to have the predicted molecular weight for hexadecameric holoenzyme (FIG. 14c) and kinetic parameters consistent with cyanobacterial Rubisco (FIG. 17a). These results clearly demonstrate that Se7942 Rubisco can be properly assembled by the tobacco chloroplast chaperones without the intervention of either cyanobacterial RbcX or CcmM35. In addition, modification of regulatory elements within the synthetic transgene operon lead to slightly enhanced Rubisco expression in SeLS plants compared to SeLSX plants. SeLS plants grow faster than other transformants and only slightly more slowly than the wild-type plants under a 3% CO₂ atmosphere (FIG. 19).

CcmM35 appears to impede the degradation of cyanobacterial Rubisco by chloroplast proteases as the leaves age (FIG. 22a). Despite greater cyanobacterial Rubisco abundance, SeLSM35 and SeLSYM35 plants do not grow more rapidly than SeLS. Although association with CcmM35 or tobacco SSU seems to have a slightly negative effect on cyanobacterial Rubisco kinetics, it is unlikely that this effect alone is responsible for the poorer growth of SeLSM35 and SeLSYM35 plants. Slower remobilization of Rubisco-CcmM35 complexes from aging leaves may also impair growth and development. We believe that organization of the cyanobacterial enzyme by CcmM35 into extensive complexes larger than 2 μm in size limits access of the substrates to active sites located in the complex interior, leading to reduced rates of photosynthesis and a commensurate underestimation of Rubisco content as determined by ¹⁴C-CABP binding. As a comparison, β-carboxysomes found in Se7942 are normally about 100-200 nm in size (Orus et al., Plant Physiol. 107:1159-1166, 1995; Cannon et al., Appl. Environ. Microbiol. 67: 5351-5361, 2001).

The chloroplast transformation technology used in the current work has the capacity to introduce multiple transgenes and appears ideal for the expression of β-carboxysomes or other CCMs in higher plant chloroplasts to improve photosynthesis (Lu et al., Proc. Natl. Acad. Sci. USA 110: E623-632, 2013). The discovery of the intercistronic expression element (IEE) has greatly facilitated the stacking of multiple transgenes in synthetic operons for reliable expression of downstream genes in the chloroplast genome (Zhou et al., Plant J. 52: 961-972, 2007; Kolotilin et al., Biotechnol. Biofuels 6: 65, 2013; Lu et al., Proc. Natl. Acad. Sci. USA 110: E623-632, 2013). Although the processing at the IEE sites was incomplete in our transformants, it is not surprising that the genes located downstream were still efficiently translated since proteins from unprocessed multigene transcripts are often successfully produced from tobacco chloroplast transformants (Quesada-Vargas et al., Plant Physiol. 138: 1746-1762, 2005; Whitney et al., Proc. Natl. Acad. Sci. USA 112: 3564-3569, 2015). Post-transcriptional control is crucial for chloroplast gene expression; genes located downstream of a large operon can still be efficiently expressed through a strong translational signal (Stern et al., Annu. Rev. Plant Biol. 61: 125-155, 2010; Hanson et al., J. Exp. Bot. 64: 731-742, 2013).

Materials and Methods

The above-described results in Example 2 were performed using the following materials and methods.

Construction of the transformation vectors. The amplifications of DNA molecules were carried out with Phusion High-Fidelity DNA polymerase (Thermo Scientific, Grand Island, New York). Table 3 (see below) contains the primers ordered from Integrated DNA Technologies (Coralville, Iowa) and used in this work. At-Trps16-IEE-SD-rbcS created in our previous work (Lin et al., Nature 513: 547-550, 2014) with overlap extension PCR was digested with MluI restriction enzyme (Thermo Scientific) and ligated in forward orientation at the MauBI site of pCT-rbcL vector (Lin et al., Nature 513: 547-550, 2014) to obtain the chloroplast transformation vector pCT-LS used in the generation of SeLS tobacco line.

TABLE 3
Oligonucleotides used in the construction of chloroplast transformation vectors, DNA
blot analyses of the tobacco chloroplast rbcL locus and RNA, blot analyses of the
transplastomic tobacco lines. The MluI restriction site is underlined.

| Primers | Nucleotide sequences |
|---|---|
| YFPfor | GTCAACAGATCTCAAGAAGGAGATATACCCATGGTTAGTAAAGGTGAAGAATTGTTTACTG (SEQ ID NO: 68) |
| YFPrev | TGAACCCCCCACCTTCCACCAGAACCTCCCCTTGTACAACTCGTCCATTCCTAAAG (SEQ ID NO: 69) |
| M35CMfor | CTGGTGGAAGTGGGGGTTCATCTGCTTATAACGGACAAGGTCG (SEQ ID NO: 70) |
| M35CMrev | AGATGCGGCCGCACGCGTTTACGGCTTCTGAATCAACAACTCAG (SEQ ID NO: 71) |
| T7-Nt-rbcLrev | GAAATTAATACGACTCACTATAGGGTTACTTATCCAAAACGTCCACTGCTG (SEQ ID NO: 72) |
| Nt-rbcLfor | ATCATATTCACTCTGGTACCGTAG (SEQ ID NO: 73) |
| T7-aadArev | GAAATTAATACGACTCACTATAGGGTTTGCCAACTACCCTTAGTGATCTC (SEQ ID NO: 74) |
| aadAfor | ATGGCTCGTGAAGCGGTTACG (SEQ ID NO: 75) |
| T7-Se-rbcLrev | GAAATTAATACGACTCACTATAGGGAGCTTATCCATTGTCTCAAATTCAAAC (SEQ ID NO: 76) |
| Se-rbcL5 | GTATGCCAATCATAATGCATGATTTTC (SEQ ID NO: 77) |
| T7-Se-rbcSrev | GAAATTAATACGACTCACTATAGGGATATCTTCCAGGTCGATGCACAATG (SEQ ID NO: 78) |
| Se-rbcS5 | GGATCCATGAGTATGAAAACCTTGCCAAAAGAACG (SEQ ID NO: 79) |
| T7-Se-rbcXrev | GAAATTATACGACTCACTATAGGGTCAATCCGCATGGGAGGCATTAG (SEQ ID NO: 80) |
| Se-rbcX5 | CGCATCAGTCGCGATACAG (SEQ ID NO: 81 ) |
| T7-Se-Mrev | GAAATTAATACGACTCACTATAGGGCGGCTTTTGAATCAACAGTTCAGC (SEQ ID NO: 82) |
| Se-M5 | TCCTGCGCACCGATTCAAAGT (SEQ ID NO: 83) |
| T7-Se- | GAAATTAATACGACTCACTATAGGGCGGCTTCTGAATCAACAACTC (SEQ ID NO: 84) |
| MCMrev | |
| Se-MCM5 | ACCTGATGGTTCGGTTCCTGAATC (SEQ ID NO: 85) |

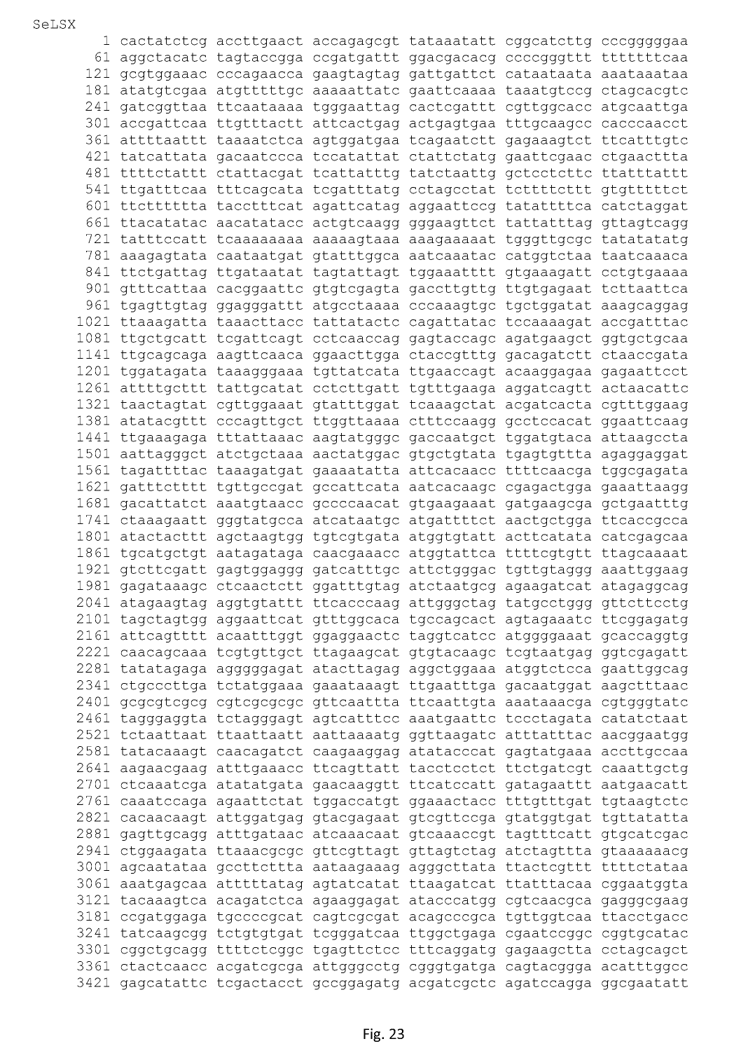

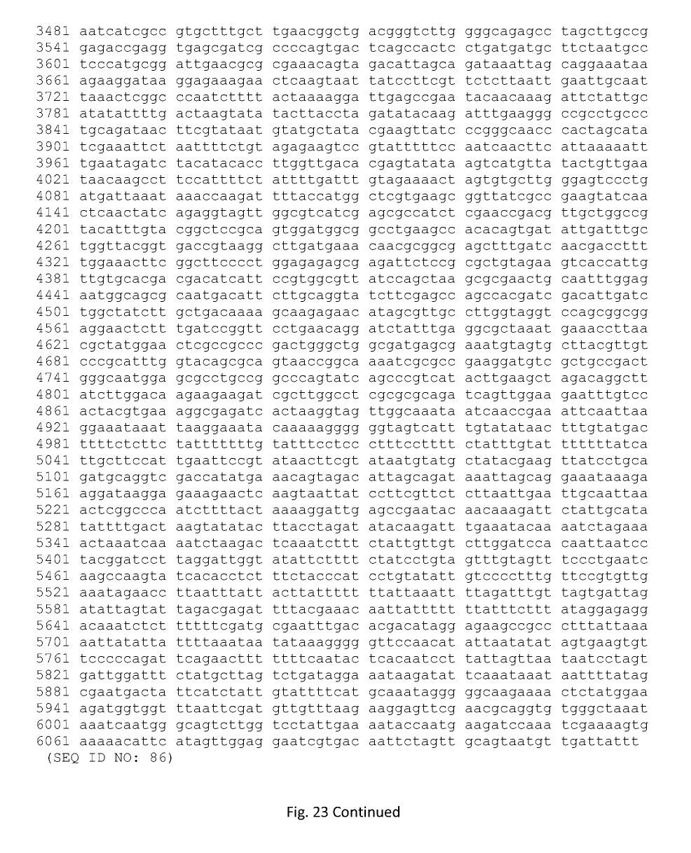

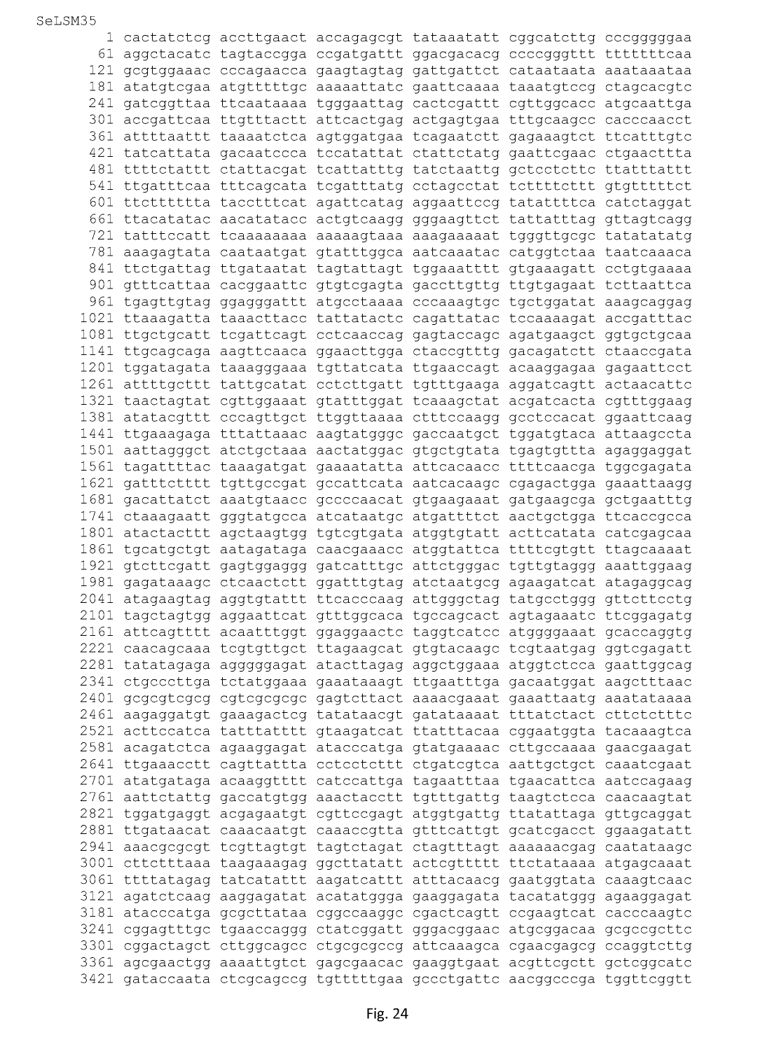

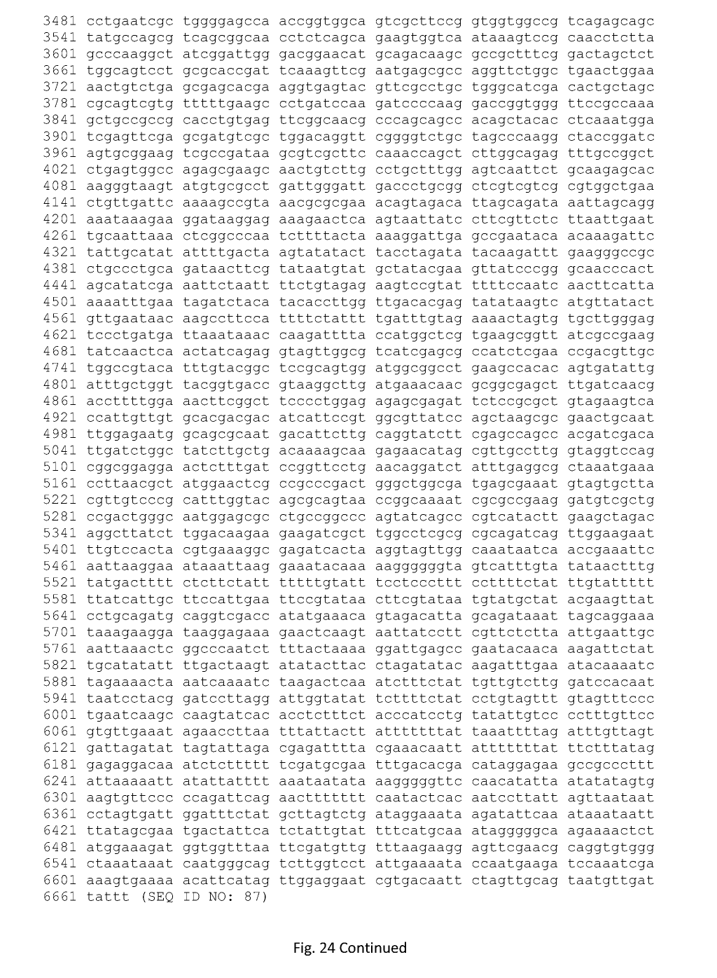

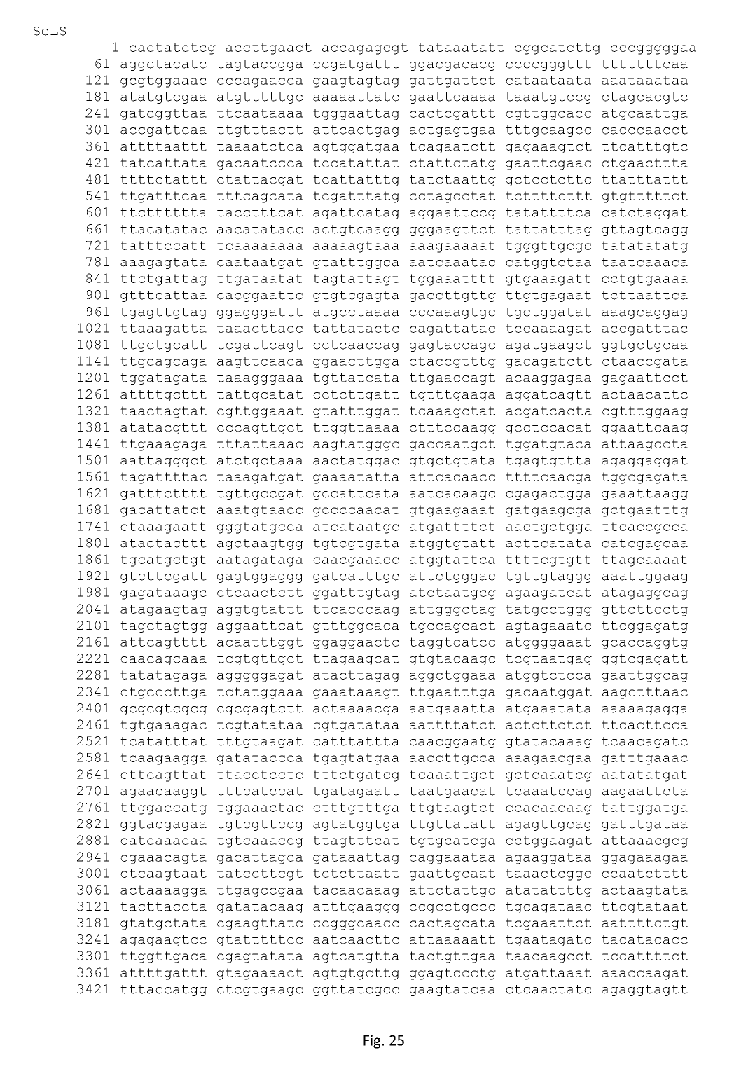

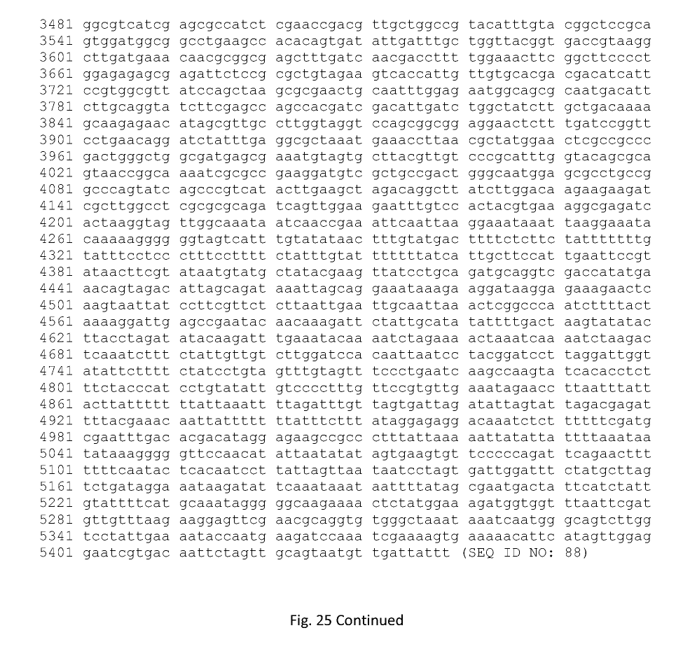

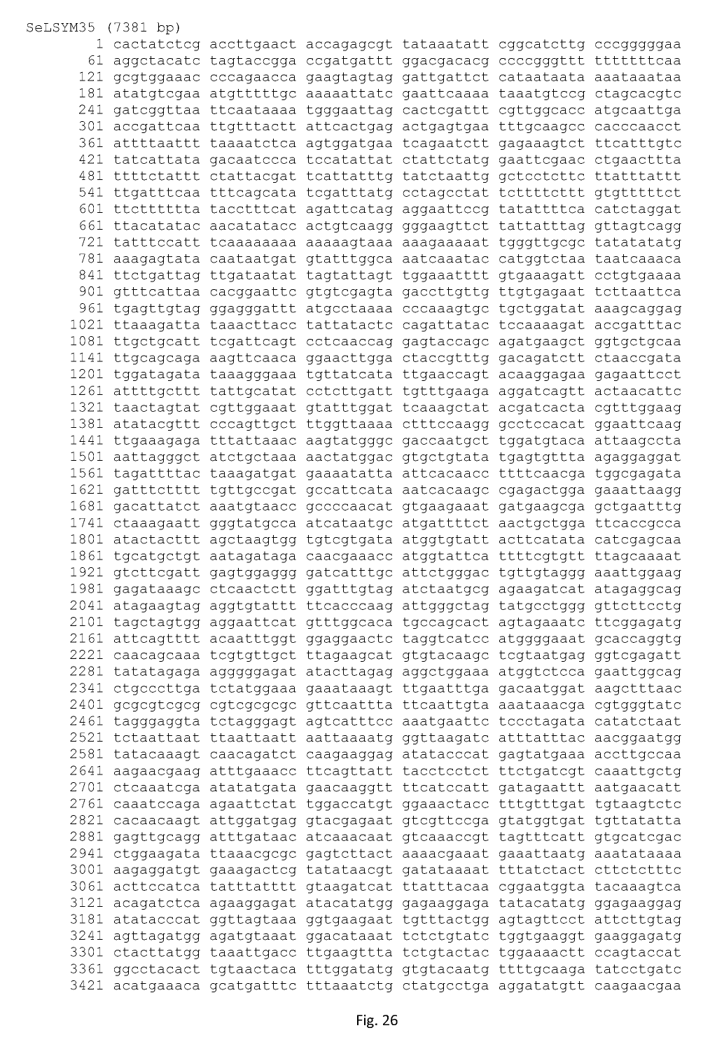

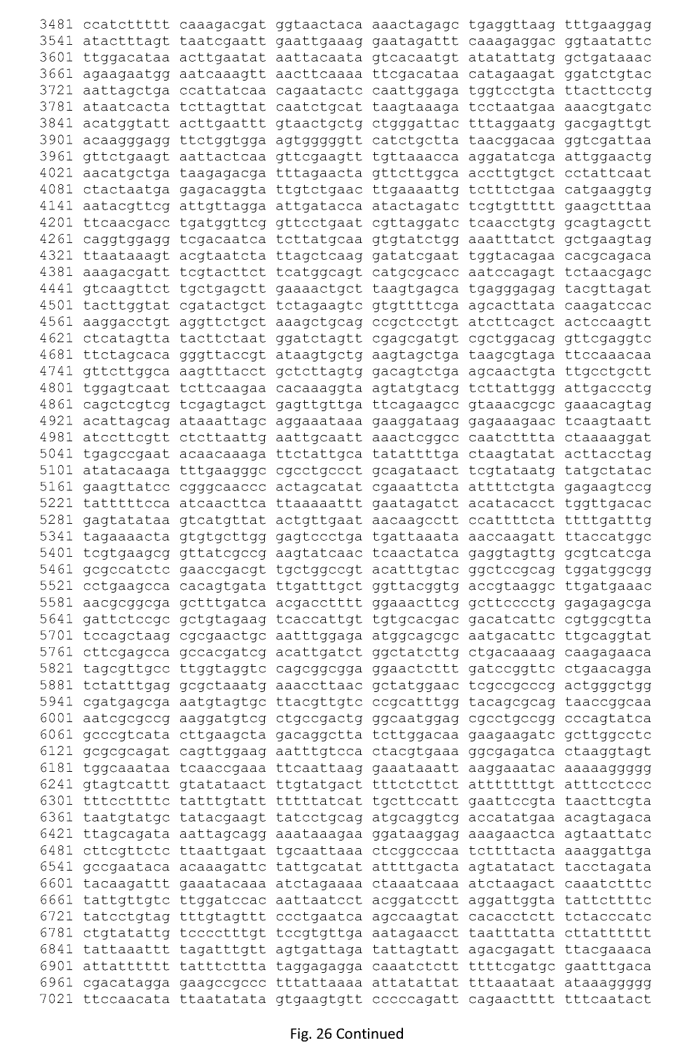

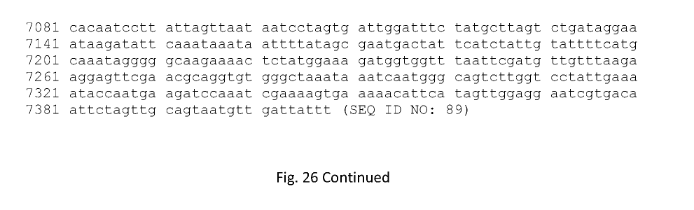

We designed the Se-ccmM35 and yfp genes with codons optimized for the chloroplast translation system (Puigbo et al., Nucleic Acids Res. 35: W126-131, 2007) and had them synthesized by Bioneer Inc. (Alameda, Calif.). We then used the primers YFPfor-YFPrev and M35CMfor-M35CMrev to amplify the yfp and Se-ccmM35 genes respectively to add 3′ end of IEE-SD sequence in front of yfp and overlaps to join yfp at the N-terminus of Se-ccmM35. We then applied the overlap extension PCR procedure to generate the At-Trps16-IEE-SD18-yfp-ccmM35 DNA fragment, which was then digested with MluI and subsequently ligated into the MauBI site of pCT-rbcL vector to create pCT-rbcL-YM35 vector. At-TpetD-IEE-SD-rbcS was digested with MluI and ligated into the MauBI site of the pCT-rbcL-YM35 vector to obtain the chloroplast transformation vector pCT-LSYM35, used in the generation of SeLSYM35 tobacco line. The procedures to generate tobacco chloroplast transformants and RNA blot analyses are described herein. FIGS. 23, 24, 25, and 26 respectively show the sequences of chloroplast transformation constructs SeLSX, SeLSM35, SeLS, and SeLSYM35.

Generation of transplastomic tobacco plants. We used the Biolistic PDS-1000/He Particle Delivery

System (Bio-Rad Laboratories, Inc.) and a tissue culture selection method (Maliga et al., Methods Mol. Biol. 1132: 147-163, 2014). Two-week-old tobacco (Nicotiana tabacum cv. Samsun) seedlings germinated in sterile MS agar medium were bombarded with 0.6 μm gold particles carrying the appropriate chloroplast transformation vector. Two days later, the leaves were cut in half and put on RMOP agar plates containing 500 mg/l of spectinomycin and incubated for 4-6 weeks at 23° C. with 14 hours of light per day. The shoots arising from this medium were cut into small pieces of about 5 mm² and subjected to the second round of regeneration in the same RMOP medium for about 4-6 weeks. If necessary, the shoots from the second round were subjected to another round of selection before they were transferred to MS agar medium containing 500 mg/I of spectinomycin for rooting and then to soil for growth in a greenhouse chamber with elevated atmospheric CO₂. DNA blot analyses with the DIG-labeled probe amplified from Nt-PrbcL region were used to determine the homoplasmy of the transformed plants as described previously (Lin et al., Nature 513: 547-550, 2014).

RNA blot analyses of the transcripts from transplastomic tobacco plants. First, we generated DNA templates with T7 promoter located on the complement strand at the end of each gene using the primers listed in Table 3. From these DNA templates, the DIG-labeled RNA probes were synthesized with MEGAshortscript kit (Ambion, Foster City, Calif.) and DIG RNA Labeling Mix (Roche Life Science). Each RNA probe was precipitated with ammonium acetate and ethanol and its concentration was determined with Qubit® RNA BR Assay Kit (Invitrogen, Carlsbad, Calif.). Generally, as little as 0.1 pg of the probe on a positively charged Nylon membrane can be detected with the alkaline phosphatase-conjugated anti-Digoxigenin and CDP-star chemiluminescent substrate (Roche Life Science).

Tissue samples were collected from fully expanded leaves from the top parts of the plants, rapidly frozen in liquid N₂ and stored at −80° C. before use. These samples were then thawed in RNAlater®-ICE Frozen Tissue Transition Solution (Life Technologies) at −20° C. Approximately 30-60 mg of each sample was homogenized in 600 μL of Lysis Buffer from PureLink® RNA Mini Kit (Life Technologies) containing 1% (v/v) 2-mercaptoethanol, and RNA extraction was carried out according to the manufacturer's protocol. The RNA concentrations were estimated with the Qubit® RNA BR Assay Kit.

For each RNA blot, 0.2 μg of each RNA sample was mixed with three volumes of NorthernMax® Formaldehyde Load Dye (Life Technologies) with 50 μg/mL of ethidium bromide, incubated at 65° C. for 15 min and separated in a 1.3% agarose denaturing gel prepared with 2% formaldehyde with MOPS buffer under an electric field strength of 7 V/cm for 2 hr. The integrity of the RNA samples in the agarose gel was examined under UV light. The gel was then equilibrated with DEPC-treated H₂O for 10 min three times, 50 mM NaOH for 20 min and 20× SSC buffer for 45 min before the RNAs were transferred to a positively charged Nylon membrane in 20× SSC under capillary action for 3-5 hr. The RNAs were then crosslinked to the membrane with UV radiation and hybridized with 100 ng of DIG-labeled RNA probe in 3.5 mL of DIG Easy Hyb buffer (Roche Life Science) at 68° C. overnight. The hybridized probe was then detected with the alkaline phosphatase-conjugated anti-Digoxigenin and CDP-star chemiluminescent substrate (Roche Life Science) according to the manufacturer's instructions.

Anatomical and biochemical characterization. Transplastomic lines and wild-type tobacco were grown in air containing 3% (v/v) (30,000 ppm) CO₂ at a light intensity of 250 pmol photons m⁻² s⁻¹ . Duration, temperature and relative humidity during the diel cycle were 16 h, 24° C., 70% and 8h, 22° C., 80% for the light and dark periods, respectively. Wild-type tobacco was also grown at normal atmospheric CO₂ (400 ppm) under the environmental conditions given above. The leaf area, leaf number and plant height were recorded every 2-3 days using three plants from each genotype. Leaf homogenates were obtained from leaf discs ((Andralojc et al., Food and Energy Security 3: 69-85, 2014) taken from the lowest non-senescent (bottom), the youngest fully-expanded (top), and mid-way between these extremes (mid) from pre-anthesis plants whose total leaf area was ˜5,000 cm². The total protein (Upreti et al., 2012) and chlorophyll content (Wintermans et al., Biochim. Biophys. Acta 109: 448-453, 1965) were determined using crude leaf homogenates (i.e. prior to centrifugation). The crude homogenates were also used to quantify Rubisco, since significant Rubisco activity was present in insoluble material from leaves expressing SeLSM35 and SeLSYM35. Soluble protein was determined (Bradford, Anal. Biochem. 72: 248-254, 1976) following homogenate centrifugation (14,250×g for 5 min at 4° C.). The leaf fresh weight and leaf dry weight (80° C. for 48 hours) were determined using leaf discs from the leaves described above.

SDS-PAGE, blue-native PAGE and immunoblot. For SDS-PAGE and blue-native PAGE, crude leaf homogenates were separated in 4-20% polyacrylamide gradient gels (Thermo Scientific, Horsham, UK) and 3-12% polyacrylamide gradient gels (Invitrogen) respectively, as described previously (Nijtmans et al., Methods 26: 327-334, 2002; Lin et al., Nature 513: 547-550, 2014). The proteins were transfer to a PVDF membrane (Immobilon-P, Millipore, Nottingham, UK) and probed with antibodies against cyanobacterial (SePCC6301) Rubisco, tobacco Rubisco, tobacco Rubisco SSU and CcmM from SePCC7942 (produced by Cambridge Research Biochemicals) as described previously (Lin et al., Nature 513: 547-550, 2014). The primary antibodies were detected using an anti-rabbit peroxidase (HRP)-conjugate and a chemiluminescent ECL substrate (Li-Cor, Cambridge, UK).

Cryo-preparation of leaf material, immunogold labelling and transmission electron microscopy. Leaf discs were cryo-fixed using a high pressure freezer EM HPM100 (Leica Microsystems) at a cooling rate of 20,000 Kelvins/sec. The cryo-fixed samples were then subjected to freeze substitution in 0.5% uranyl acetate in dry acetone using an EM AFS unit (Leica Microsystems) and polymerized in Lowicryl HM20 resin (Polysciences) as described previously (Lin et al., Plant J. 79:1-12, 2014).

Ultrathin sections (60-90 nm) of embedded leaf material were subjected to immunogold labelling as describe previously (Lin et al., Plant J. 79:1-12, 2014). Four primary antibodies against CcmM35, cyanobacterial Rubisco (from Synechococcus elongatus PCC6301), tobacco Rubisco and tobacco Rubisco SSU were used. The primary antibodies were detected using a secondary (goat anti-rabbit) antibody conjugated with 10 nm gold particles (Abcam UK, ab39601). Micrographs were taken using a Jeol 2011 F transmission electron microscope operating at 200 kV, equipped with a Gatan Ultrascan CCD camera and a Gatan Dual Vision CCD camera.

Rubisco purification. For Vₘₐₓ^{C}, Vₘₐₓ^{O}, K_{M}^{C} and K_{M}^{O} determination, leaf tissue was homogenized in assay buffer (100 mM Bicine-NaOH pH 8.0; 10 mM MgCl₂; 1 mM EDTA; 1 mM ε-aminocaproic acid; 1 mM benzamidine; plant protease inhibitor cocktail (diluted 1:100, Sigma); 1 mM PMSF; 1 mM KH₂PO₄; 2% (w/v) PEG-4000; 10 mM NaHCO₃; 10 mM DTT; insoluble PVP (150 mg/gFW)) and was used immediately after centrifugation (2 min, 290×g). This approach gave results very similar to those obtained when a higher speed centrifugation and G-25 Sephadex desalting was included, prior to assay (Andralojc et al., Food and Energy Security 3: 69-85, 2014). The tendency of Rubisco co-expressed with ccmM35 or ccmYM35 to sediment necessitated this simplified approach. Parallel controls demonstrated that the resulting carboxylase activities were entirely RuBP-dependent and were fully inhibited by prior treatment with the Rubisco inhibitor, 2-carboxy-D-arabinitol-1,5-bisphosphate (CABP).

For specificity factor determination, leaf material was homogenized in extraction buffer (40 mM TEA (pH 8, HCl), 10 mM MgCl₂, 0.5 mM EDTA, 1 mM K₂HPO₄, 1 mM ε-aminocaproic, 1 mM benzamidine, 50 mM 2-mercaptoethanol, 5 mM DTT, 10 mM NaHCO₃, 1 mM PMSF, 1% (v/v) TX-100 and 1% (w/v) insoluble PVP) and purified using DEAE Sephacel (Pharmacia), a subsequent cycle of anion-exchange chromatography with gradient elution (HiTrap 0, GE-Healthcare), and concentration to ˜20 mg Rubisco. mL⁻¹ using Ultra-15 centrifugal filter devices (AMICON).

Rubisco activity assay. The determination of Vₘₐₓ^{C}, Vₘₐₓ^{O}, K_{M}^{C} and K_{M}^{O} was performed at 25° C. in solutions equilibrated with oxygen-free nitrogen or air (79% N₂, 21% 0₂) containing 200 mM Bicine-NaOH pH 8.1, 20 mM MgCl₂, 0.4 mM RuBP, 70 units. mL⁻¹ carbonic anhydrase and six different concentrations of sodium bicarbonate (3.7×10¹⁰ Bq mol⁻¹) to provide CO₂ concentrations of 7-550 μM and 1.4-110 μM for cyanobacterial and tobacco Rubisco respectively.

The K_{M}^{O} was obtained from the relationship: K_{M}^{C} (at stated O₂ concentration)=K_{M}^{C} (in N₂)*(1+([O₂]/K_{M}^{O}).

The Vₘₐₓ^{O} was obtained from the equation, S_{c/c}=[Vₘₐₓ^{C}/K_{M}^{C}]/[Vₘₐₓ^{O}/K_{M}^{O}]. The concentration of Rubisco catalytic sites was determined by the [¹⁴C]CABP binding method (Yokota and Canvin, 1985) using [2¹-¹⁴C]CABP (3.7×10¹⁰ Bq mol⁻¹ and 3.7×10¹¹ Bq mol⁻¹ for WT and transplastomic tobacco samples, respectively). The specificity factor (S_{c/c}) was determined at 25° C. by monitoring the total consumption of RuBP in an oxygen electrode, as described previously (Parry et al., J. Exp. Bot. 40: 317-320, 1989).

Gas-exchange measurements. The gas exchange measurements were performed using a LI-6400XT portable photosynthesis system (LiCor, Lincoln, Nebr., USA) at constant irradiance (1,000 μmol photons. m⁻². s⁻¹), 25 ±0.5° C., a vapour pressure deficit of 0.8-1.0 kPa, a flow rate of 200 μmol s⁻¹ with CO₂ concentrations ranging between 100 and 2,000 μmol. mol air⁻¹. The A-Ci curves were determined under photorespiratory and non-photorespiratory conditions, using air containing 21% and 2% (v/v) O₂ respectively. The results were related to both leaf area and Rubisco active site concentration, the latter determined by [¹⁴C]-CABP binding assay (Yokota et al., Plant Physiol. 77: 735-739, 1985).

Uses The development of plants that are genetically engineered to produce a more efficient form of Rubisco, such as employing a cyanobacterial Rubisco, is useful for increasing crop yields.

Other Embodiments All publications mentioned in the above specification are hereby incorporated by reference. Various modifications and variations of the described methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

What is claimed is:

1. A plant comprising a cyanobacterial ribulose-1,5,-bisphosphate carboxylase/oxygenase (Rubisco) which can assemble and fix carbon without an interacting protein.

2. The plant of claim 1, wherein said interacting protein is rbcX or CcmM35.

3. The plant of claim 1, wherein said plant is a C3 plant.

4. The plant of claim 1, wherein said cyanobacterial Rubisco assembles and fixes carbon in the cytoplasm of a plant cell.

5. The plant of claim 4, wherein said cyanobacterial Rubisco assembles and fixes carbon in a microcompartment in the cytoplasm.

6. The plant of claim 1, wherein said cyanobacterial Rubisco assembles and fixes carbon in a chloroplast of a plant cell.

7. The plant of claim 6, wherein said cyanobacterial Rubisco assembles and fixes carbon in a microcompartment in the chloroplast.

8. A method of expressing a cyanobacterial Rubisco in a plant cell, said method comprising expressing a cyanobacterial large Rubisco subunit and a cyanobacterial small Rubisco subunit in said plant cell which can assemble and fix carbon without an interacting protein.

9. The method of claim 8, wherein said interacting protein is rbcX or CcmM35.

10. The method of claim 8, wherein said plant is a C3 plant.

11. The method of claim 8, wherein said cyanobacterial Rubisco assembles and fixes carbon in the cytoplasm of a plant cell.

12. The method of claim 11, wherein said cyanobacterial Rubisco assembles and fixes carbon in a microcompartment in the cytoplasm.

13. The method of claim 8, wherein said cyanobacterial Rubisco assembles and fixes carbon in a chloroplast of a plant cell.

14. The method of claim 13, wherein said cyanobacterial Rubisco assembles and fixes carbon in a microcompartment in the chloroplast.

15. A method of engineering a plant expressing a cyanobacterial Rubisco, said method comprising (a) providing a plant cell that expresses a polypeptide having substantial identity to a cyanobacterial Rubisco large subunit and a polypeptide having substantial identity to a cyanobacterial Rubisco small subunit which can assemble and fix carbon without an interacting protein; and (b) regenerating a plant from said plant cell wherein plant expresses said cyanobacterial Rubisco compared to a corresponding untransformed plant.

16. The method of claim 15, wherein said interacting protein is rbcX or CcmM35.

17. The method of claim 15, wherein said plant is a C3 plant.

18. The method of claim 15, wherein said cyanobacterial Rubisco assembles and fixes carbon in the cytoplasm of a plant cell.

19. The method of claim 18, wherein said cyanobacterial Rubisco assembles and fixes carbon in a microcompartment in the cytoplasm.

20. The method of claim 15, wherein said cyanobacterial Rubisco assembles and fixes carbon in a chloroplast of a plant cell.

21. The method of claim 20, wherein said cyanobacterial Rubisco assembles and fixes carbon in a microcompartment in the chloroplast.

22. A plant comprising a red-type Rubisco which can assemble and fix carbon without an interacting protein.

23. A plant comprising a Halothiobacillus Rubisco which can assemble and fix carbon without an interacting protein.

24. A plant comprising a Procholorococcus Rubisco which can assemble and fix carbon without an interacting protein.

25. A plant comprising a Synechoccocus Rubisco which can assemble and fix carbon without an interacting protein.

26. A plant comprising a Rhodobacter Rubisco which can assemble and fix carbon without an interacting protein.

27. A plant comprising a Limonium gibertii Rubisco which can assemble and fix carbon without an interacting protein.

28. A plant according to any one of claims 22-27, wherein said plant is a C3 plant.

29. A plant according to any one of claims 22-28, wherein said Rubisco is housed in a microcompartment in a chloroplast of the plant.
